SEQUENCE LISTING (46 sequences)

SEQ ID NO: 1 - DNA, 1440 bp, Phascolarctobacterium faecium
gacgaacgctggcggcgtgcctaacacatgcaagtcgaacggagaattttatttcggtagaattcttagtggcgaacgggtgagtaacgcgtaggcaacctaccctttagacggggacaacattccgaaaggagtgctaataccggatgtgatcatcttgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagatttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaaccta

SEQ ID NO: 2 - DNA, 1392 bp, Fusobacterium ulcerans
gatgaacgctgacagaatgcttaacacatgcaagtctacttgatccttcgggtgaaggtggcggacgggtgagtaacgcgtaaagaacttgccttacagactgggacaacatttggaaacgaatgctaataccggatattatgattgggtcgcatgatctgattatgaaagctatatgcgctgtgagagagctttgcgtcccattagttagttggtgaggtaacggctcaccaagacgatgatgggtagccggcctgagagggtgaacggccacaaggggactgagacacggcccttactcctacgggaggcagcagtggggaatattggacaatggaccaaaagtctgatccagcaattctgtgtgcacgaagaagtttttcggaatgtaaagtgctttcagttgggaagaagtcagtgacggtaccaacagaagaagcgacggctaaatacgtgccagcagccgcggtaatacgtatgtcgcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcccaagaagttaacagagatgttttcgtgcctcttcggaggaacttggtgacaggtggtgcatggctgtcgtcagctcgtgtcgtgagatgttgggttaagtcccgcaacgagcgcaacccctttcgtatgttaccatcattaagttggggactcatgcgagactgcctgcgatgagcaggaggaaggtggggatgacgtcaagtcatcatgccccttatacgctgggctacacacgtgctacaatgggtagtacagagagctgcaaacctgcgagggtaagctaatctcataaaactattcttagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtaacaggcctaaccgtaa

SEQ ID NO: 3 - DNA, 1443 bp, Bacteroides dorei
  misc_feature (7)..(9): n is a, c, g, or t
agtttgnnntatggctcaggatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggat

SEQ ID NO: 4 - DNA, 1420 bp, Bacteroides uniformis
gatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcctgctggactgtaactgacgctgatgctcgaaagtgtgggtatcaaacaggattagataccctggtagtccacaccagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggtgatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggag

SEQ ID NO: 5 - DNA, 1410 bp, Subdoligranulum sp. 4
gacgaacgctggcggcgcgcctaacacatgcaagtcgaacggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcggatgcatacctaagagattagggaagtccttcgggacatccagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgataccgcgaggtggagcaaacctcacaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgc

SEQ ID NO: 6 - DNA, 1418 bp, Paraprevotella xylaniphila
  misc_feature (180)..(180): n is a, c, g, or t
  misc_feature (1411)..(1412): n is a, c, g, or t
gatgaacgctagctacaggcttaacacatgcaagtcgaggggcagcatgaacttagcttgctaagtttgatggcgaccggcgcacgggtgagtaacgcgtatccaacctgccctttacccggggatagccttctgaaaaggaagtttaatacccgatgaattcgtttagtcgcatggctngatgaataaagattaattggtaaaggatggggatgcgtcccattagcttgttggcggggtaacggcccaccaaggcgacgatgggtaggggttctgagaggaaggtcccccacattggaactgagacacggtccaaactcctacgggaggcagcagtgaggaatattggtcaatgggcgcgagcctgaaccagccaagtagcgtggaggacgacggccctacgggttgtaaactccttttataaggggataaagttggccatgtatggccatttgcaggtaccttatgaataagcatcggctaattccgtgccagcagccgcggtaatacggaagatgcgagcgttatccggatttattgggtttaaagggagcgtaggcgggctgtcaagtcagcggtcaaatggcgcggctcaaccgcgttccgccgttgaaactggcagccttgagtatgcacagggtacatggaattcgtggtgtagcggtgaaatgcttagatatcacgaggaactccgatcgcgcaggcattgtaccggggcattactgacgctgaggctcgaaggtgcgggtatcaaacaggattagataccctggtagtccgcacagtaaacgatgaatgcccgctgtcggcgacatagtgtcggcggccaagcgaaagcgttaagcattccacctggggagtacgccggcaacggtgaaactcaaaggaattgacgggggcccgcacaagcggaggaacatgtggtttaattcgatgatacgcgaggaaccttacccgggcttgaatcgcaggtgcatgggccggagacggccctttccttcgggactcctgcgaaggtgctgcatggttgtcgtcagctcgtgccgtgaggtgtcggcttaagtgccataacgagcgcaacccccctccccagttgccaccgggtaatgccgggcactttggggacactgccaccgcaaggtgcgaggaaggtggggatgacgtcaaatcagcacggcccttacgtccggggcgacacacgtgttacaatggggggtacagagggccgctgcccggtgacggttggccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtgcctgaagtccgtnnccgcga

SEQ ID NO: 7 - DNA, 1419 bp, Parabacteroides johnsonii
gatgaacgctagcgacaggcttaacacatgcaagtcgaggggcagcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggttttctagcaatagctgattacgaggtgctgcatggttgtcgtcagctcgtgccgtgaggtgtcggcttaagtgccataacgagcgcaacccttatcactagttactaacaggttaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaa

SEQ ID NO: 8 - DNA, 1414 bp, Alistipes sp. JC136
  misc_feature (699)..(699): n is a, c, g, or t
  misc_feature (731)..(731): n is a, c, g, or t
gatgaacgctagcggcaggcctaacacatgcaagtcgaggggcagcgggattgaagcttgcttcagttgccggcgaccggcgcacgggtgcgtaacgcgtatgcaacctacccataacagggggataacactgagaaatcggtactaatatcccataacatcaagaggggcatcccttttggttgaaaactccggtggttatggatgggcatgcgttgtattagctagttggtgaggtaacggctcaccaaggcgacgatacatagggggactgagaggttaaccccccacattggtactgagacacggaccaaactcctacgggaggcagcagtgaggaatattggtcaatggacgcaagtctgaaccagccatgccgcgtgcaggatgacggctctatgagttgtaaactgcttttgtacgagggtaaacccggatacgtgtatccggctgaaagtatcgtacgaataaggatcggctaactccgtgccagcagccgcggtaatacggaggattcaagcgttatccggatttattgggtttaaagggtgcgtaggcggtttgataagttagaggtgaaataccggtgcttaacaccggaactgcctctaatactgttgagctagagagtagttgcggtaggcggaatgtatggtgtagcggtgaaatgcttagagatcatacagaacaccgattgcngaaggcagcttaccaaactatatctgacgttngaggcacgaaagcgtgggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgac

SEQ ID NO: 9 - DNA, 1416 bp, Parabacteroides gordonii
  misc_feature (1408)..(1408): n is a, c, g, or t
gatgaacgctagcgacaggcttaacacatgcaagtcgaggggcagcaggaagtagcaatactttgctggcgaccggcgcacgggtgagtaacgcgtatgcaacctacctatcagagggggataacccggcgaaagtcggactaataccgcataaaacaggggtcccgcatgggaatatttgttaaagatttattgctgatagatgggcatgcgttccattagatagttggtgaggtaacggctcaccaagtcttcgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggttcgtaaacttcttttataggggaataaagtgcaggacgtgtcctgttttgtatgtaccctatgaataaggatcggctaactccgtgccagcagccgcggtaatacggaggatccgagcgttatccggatttattgggtttaaagggtgcgtaggtggctttttaagtcagcggtgaaagtttgtggctcaaccataaaattgccgttgaaactggagggcttgagtatatttgaggtaggcggaatgcgtggtgtagcggtgaaatgcatagatatcacgcagaactccaattgcgaaggcagcttactaaactataactgacactgaagcacgaaagcgtggggatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtaagttgaccggagtggaaacactctttctagcaatagcaatttacgaggtgctgcatggttgtcgtcagctcgtgccgtgaggtgtcggcttaagtgccataacgagcgcaacccttatctttagttactaacaggtcgagctgaggactctaaagagactgccagcgtaagctgtgaggaaggtggggatgacgtcaaatcagcacggcccttacatccggggcgacacacgtgttacaatggtggggacaaagggcagctacctggcgacaggatgctaatctccaaaccccatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtnaccgcaag

SEQ ID NO: 10 - DNA, 1419 bp, Eubacterium limosum
gacgaacgctggcggtatgcttaacacatgcaagtcgaacgagaaggttttgatggatccttcgggtgacattagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaagaatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggacgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgtaaggactcagcagt

SEQ ID NO: 11 - DNA, 1413 bp, Parabacteroides distasonis
  misc_feature (1387)..(1387): n is a, c, g, or t
gatgaacgctagcgacaggcttaacacatgcaagtcgaggggcagcacaggtagcaataccgggtggcgaccggcgcacgggtgagtaacgcgtatgcaacttgcctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccnggggtacctgaagtccgtaaccgcga

SEQ ID NO: 12 - DNA, 25 bp, Fusobacterium ulcerans
tgcactgttggattttctaaaaagg

SEQ ID NO: 13 - DNA, 20 bp, Fusobacterium ulcerans
actttgggcatgctaaacca

SEQ ID NO: 14 - DNA, 20 bp, Bacteroides dorei
cgccggatgcatatacaaga

SEQ ID NO: 15 - DNA, 20 bp, Bacteroides dorei
tctcgccaatgatgtccaaa

SEQ ID NO: 16 - DNA, 23 bp, Eubacterium limosum
ccgcacaagagaaataaacgcca

SEQ ID NO: 17 - DNA, 23 bp, Eubacterium limosum
tggcaaattcaaaggtgagcgaa

SEQ ID NO: 18 - DNA, 24 bp, Alistipes senegalensis
gggaataaagctgttccgatatgc

SEQ ID NO: 19 - DNA, 22 bp, Alistipes senegalensis
tcatgcaacattctttcgttgg

SEQ ID NO: 20 - DNA, 25 bp, Parabacteroides gordonii
ttcaccttctacggctactactacg

SEQ ID NO: 21 - DNA, 24 bp, Parabacteroides gordonii
acataacgatcaagggtgctgaag

SEQ ID NO: 22 - DNA, 23 bp, Paraprevotella xylaniphila
gctctttttagcctgtatccggt

SEQ ID NO: 23 - DNA, 22 bp, Paraprevotella xylaniphila
atacgatacgaacgaccaacct

SEQ ID NO: 24 - DNA, 20 bp, Ruthenibacterium lactatiformans
taccaatgcaaagcgaccaa

SEQ ID NO: 25 - DNA, 20 bp, Ruthenibacterium lactatiformans
cggttttgttgccgaactct

SEQ ID NO: 26 - DNA, 21 bp, Parabacteriodes johnsonii
gcacagattctacactcccct

SEQ ID NO: 27 - DNA, 20 bp, Parabacteriodes johnsonii
agcaacgaaacaacctgtga

SEQ ID NO: 28 - DNA, 20 bp, Bacteroides uniformis
tccatgctgaagcgttgaag

SEQ ID NO: 29 - DNA, 20 bp, Bacteroides uniformis
ggaccgaacatcccaatcac

SEQ ID NO: 30 - DNA, 20 bp, Phascolarctobacterium succinatutens
ctgcttccgacagcacacat

SEQ ID NO: 31 - DNA, 20 bp, Phascolarctobacterium succinatutens
agcttggcggagagcctatt

SEQ ID NO: 32 - DNA, 22 bp, Parabacteroides distasonis
agcgcgtaaacttagtcaagga

SEQ ID NO: 33 - DNA, 20 bp, Parabacteroides distasonis
tagggccaaaacctgcatta

SEQ ID NO: 34 - DNA, 19 bp, Lactobacillus acidophilus
agcagtagggaatcttcca

SEQ ID NO: 35 - DNA, 17 bp, Lactobacillus acidophilus
caccgctacacatggag

SEQ ID NO: 36 - DNA, 1557 bp, Phascolarctobacterium faecium
attggagagtttgatcctggctcaggacgaacgctggcggcgtgcctaacacatgcaagtcgaacggagaattttatttcggtagaattcttagtggcgaacgggtgagtaacgcgtaggcaacctaccctttagacggggacaacattccgaaaggagtgctaataccggatgtgatcatcttgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagatttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaacctattaggagccagccgtctaaggtggggccgatgattggggtgaagtcgtaacaaggtagccgtatcggaaggtgcggctggatcacctccttt

SEQ ID NO: 37 - DNA, 1503 bp, Fusobacterium varium
cgaagagtttgatcctggctcaggatgaacgctgacagaatgcttaacacatgcaagtctacttgatccttcgggtgaaggtggcggacgggtgagtaacgcgtaaagaacttgccttacagactgggacaacatttggaaacgaatgctaataccggatattatgattgggtcgcatgatctgattatgaaagctatatgcgctgtgagagagctttgcgtcccattagttagttggtgaggtaacggctcaccaagacgatgatgggtagccggcctgagagggtgaacggccacaaggggactgagacacggcccttactcctacgggaggcagcagtggggaatattggacaatggaccaaaagtctgatccagcaattctgtgtgcacgaagaagtttttcggaatgtaaagtgctttcagttgggaagaagtcagtgacggtaccaacagaagaagcgacggctaaatacgtgccagcagccgcggtaatacgtatgtcgcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcccaagaagttaacagagatgttttcgtgcctcttcggaggaacttggtgacaggtggtgcatggctgtcgtcagctcgtgtcgtgagatgttgggttaagtcccgcaacgagcgcaacccctttcgtatgttaccatcattaagttggggactcatgcgagactgcctgcgatgagcaggaggaaggtggggatgacgtcaagtcatcatgccccttatacgctgggctacacacgtgctacaatgggtagtacagagagctgcaaacctgcgagggtaagctaatctcataaaactattcttagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtaacaggcctaaccgtaaggagggatgttccgagggtgtgattagcgattggggtgaagtcgtaacaaggtatccgtacgggaacgtgcggatggatcacctcctt

SEQ ID NO: 38 - DNA, 1528 bp, Bacteroides dorei
atgaagagtttgatcctggctcaggatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggatcgccctagggtaaaactggtgactggggctaagtcgtaacaaggtagccgtaccggaaggtgcggctggaacacctcctt

SEQ ID NO: 39 - DNA, 1523 bp, Bacteroides uniformis
atgaagagtttgatcctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtggaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcctgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggtgatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggctaagtcgtaacaaggtagccgtaccggaaggtgcggctggaacacctcctt

SEQ ID NO: 40 - DNA, 1526 bp, Gemminger formicilis
aatgaagagtttgatcctggctcaggacgaacgctggcggcgcgcctaacacatgcaagtcgaacggagctgttttctctgaagttttcggatggaagagagttcagcttagtggcgaacgggtgagtaacacgtgagcaacctgcctttcagtgggggacaacatttggaaacgaatgctaataccgcataagaccacagtgtcgcatggcacaggggtcaaaggatttatccgctgaaagatgggctcgcgtccgattagctagatggtgaggtaacggcccaccatggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgtcccaggggacgataatgacggtaccctgggaggaagcaccggctaactacgtgccagcagccgcggtaaaacgtagggtgcaagcgttgtccggaattactgggtgtaaagggagcgcaggcggattggcaagttgggagtgaaatctatgggctcaacccataaattgctttcaaaactgtcagtcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccctggtagtccatgccgtaaacgatgattactaggtgtgggaggattgaccccttccgtgccgcagttaacacaataagtaatccacctggggagtacgaccgcaaggttgaaactcaaaggaattgacgggggcccgcacaagcagtggagtatgtggtttaattcgaagcaacgcgaagaaccttaccaggtcttgacatcggatgcatacctaagagattagggaagtccttcgggacatccagacaggtggtgcatggttgtcgtcagctcgtgtcgtgagatgttgggttaagtcccgcaacgagcgcaacccttatcgttagttactacgcaagaggactctagcgagactgccgttgacaaaacggaggaaggtggggatgacgtcaaatcatcatgccctttatgacctgggctacacacgtactacaatggctattaacagagagaagcgataccgcgaggtggagcaaacctcacaaaaatagtctcagttcggatcgcaggctgcaacccgcctgcgtgaagccggaattgctagtaatcgcggatcagcatgccgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtatcggaaggtgcggctggatcacctccttt

SEQ ID NO: 41 - DNA, 1319 bp, Paraprevotella xylaniphila
aataaagattaattggtaaaggatggggatgcgtcccattagcttgttggcggggtaacggcccaccaaggcgacgatgggtaggggttctgagaggaaggtcccccacattggaactgagacacggtccaaactcctacgggaggcagcagtgaggaatattggtcaatgggcgcgagcctgaaccagccaagtagcgtggaggacgacggccctacgggttgtaaactccttttataaggggataaagttggccatgtatggccatttgcaggtaccttatgaataagcatcggctaattccgtgccagcagccgcggtaatacggaagatgcgagcgttatccggatttattgggtttaaagggagcgtaggcgggcagtcaagtcagcggtcaaatggcgcggctcaaccgcgttccgccgttgaaactggcagccttgagtatgcacagggtacatggaattcgtggtgtagcggtgaaatgcttagatatcacgaggaactccgatcgcgcaggcattgtaccggggcattactgacgctgaggctcgaaggtgcgggtatcaaacaggattagataccctggtagtccgcacagtaaacgatgaatgcccgctgtcggcgacatagtgtcggcggccaagcgaaagcgttaagcattccacctggggagtacgccggcaacggtgaaactcaaaggaattgacgggggcccgcacaagcggaggaacatgtggtttaattcgatgatacgcgaggaaccttacccgggcttgaatcgcaggtgcatgggccggagacggccctttccttcgggactcctgcgaaggtgctgcatggttgtcgtcagctcgtgccgtgaggtgtcggcttaagtgccataacgagcgcaacccccctccccagttgccaccgggtaatgccgggcactttggggacactgccaccgcaaggtgcgaggaaggtggggatgacgtcaaatcagcacggcccttacgtccggggcgacacacgtgttacaatggggggtacagagggccgctgcccggtgacggttggccaatccctaaaacccctctcagttcggactggagtctgcaacccgactccacgaagctggattcgctagtaatcgcgcatcagccatggcgcggtgaatacgttcccgggccttgtacacaccgcccgtcaagccatgaaagccgggggtgcctgaagtccgtgaccgcgagggtcggcctagggtaaaaccggtgattggggctaagtcgtaacaaggtagccgtaccggaaggtgcggctggaacacctccttt

SEQ ID NO: 42 - DNA, 1526 bp, Parabacteroides johnsonii
cgaagagtttgatcctggctcaggatgaacgctagcgacaggcttaacacatgcaagtcgaggggcatcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggttttctagcaatagctgattacgaggtgctgcatggttgtcgtcagctcgtgccgtgaggtgtcggcttaagtgccataacgagcgcaacccttatcactagttactaacaggttaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctcctt

SEQ ID NO: 43 - DNA, 1525 bp, Alistipes senegalensis
atggagagtttgatcctggctcaggatgaacgctagcggcaggcctaacacatgcaagtcgaggggcagcgggattgaagcttgcttcagttgccggcgaccggcgcacgggtgcgtaacgcgtatgcaacctacccataacagggggataacactgagaaatcggtactaatatcccataacatcaagaggggcatcccttttggttgaaaactccggtggttatggatgggcatgcgttgtattagctagttggtgaggtaacggctcaccaaggcgacgatacatagggggactgagaggttaaccccccacattggtactgagacacggaccaaactcctacgggaggcagcagtgaggaatattggtcaatggacgcaagtctgaaccagccatgccgcgtgcaggatgacggctctatgagttgtaaactgcttttgtacgagggtaaacccggatacgtgtatccggctgaaagtatcgtacgaataaggatcggctaactccgtgccagcagccgcggtaatacggaggattcaagcgttatccggatttattgggtttaaagggtgcgtaggcggtttgataagttagaggtgaaataccggtgcttaacaccggaactgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggtggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacctccttt

SEQ ID NO: 44 - DNA, 1523 bp, Parabacteroides gordonii
cgaagagtttgatcctggctcaggatgaacgctagcgacaggcttaacacatgcaagtcgaggggcagcaggaagtagcaatactttgctggcgaccggcgcacgggtgagtaacgcgtatgcaacctacctatcagagggggataacccggcgaaagtcggactaataccgcataaaacaggggtcccgcatgggaatatttgttaaagatttattgctgatagatgggcatgcgttccattagatagttggtgaggtaacggctcaccaagtcttcgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggttcgtaaacttcttttataggggaataaagtgcaggacgtgtcctgttttgtatgtaccctatgaataaggatcggctaactccgtgccagcagccgcggtaatacggaggatccgagcgttatccggatttattgggtttaaagggtgcgtaggtggctttttaagtcagcggtgaaagtttgtggctcaaccataaaattgccgttgaaactggagggcttgagtatatttgaggtaggcggaatgcgtggtgtagcggtgaaatgcatagatatcacgcagaactccaattgcgaaggcagcttactaaactataactgacactgaagcacgaaagcgtggggatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtaagttgaccggagtggaaacactctttctagcaatagcaatttacgaggtgctgcatggttgtcgtcagctcgtgccgtgaggtgtcggcttaagtgccataacgagcgcaacccttatctttagttactaacaggtcgagctgaggactctaaagagactgccagcgtaagctgtgaggaaggtggggatgacgtcaaatcagcacggcccttacatccggggcgacacacgtgttacaatggtggggacaaagggcagctacctggcgacaggatgctaatctccaaaccccatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtagccgtaccggaaggtgcggctggaacacctccttt

SEQ ID NO: 45 - DNA, 1521 bp, Eubacterium limosum
tattgagagtttgatcctggctcaggacgaacgctggcggtatgcttaacacatgcaagtcgaacgagaaggttttgatggatccttcgggtgatatcagaactggaaagtggcgaacgggtgagtaacgcgtgggtaacctgccctatggaaaggaatagcctcgggaaactgggagtaaagccttatattatggttttgtcgcatggcaagatcatgaaaactccggtgccataggatggacccgcgtcccattagctagttggtgagataacagcccaccaaggcgacgatgggtaaccggtctgagagggcgaacggtcacactggaactgagacacggtccagactcctacgggaggcagcagtggggaatattgcgcaatgggggcaaccctgacgcagcaataccgcgtgagtgaagaaggttttcggatcgtaaagctctgttattggggaagaagaatgacggtacccaatgaggaagtcccggctaactacgtgccagcagccgcggtaatacgtaggggacaagcgttgtccggaatgactgggcgtaaagggcgcgtaggcggtctattaagtctgatgtgaaaggtaccggctcaaccggtgaagtgcattggaaactggtagacttgagtattggagaggcaagtggaattcctagtgtagcggtgaaatgcgtagatattaggaggaacaccagtggcgaaggcggcttgctggacaaatactgacgctgaggtgcgaaagcgtggggagcgaacaggattagataccctggtagtccacgccgtaaacgatgaatgctaggtgttggggaaactcagtgccgcagttaacacaataagcattccgcctggggagtacgaccgcaaggttgaaactcaaaggaattgacggggacccgcacaagcagcggagcatgtggtttaattcgaagcaacgcgaagaaccttaccaggtcttgacatcctctgacgagcctagagataggaagtttccttcgggaacagagagacaggtggtgcatggttgtcgtcagctcgtgtcgtgagatgttgggttaagtcccgcaacgagcgcaacccctgcctttagttgccagcattaagttgggcactctagagggactgccgtagacaatacggaggaaggtggggacgacgtcaaatcatcatgccccttatgacctgggctacacacgtgctacaatggtctgaacagagggccgcgaagccgcgaggtgaagcaaatcccttaaaacagatcccagttcggattgcaggctgcaactcgcctgcatgaagttggagttgctagtaatcgcggatcagaatgccgcggtgaatgcgttcccgggtcttgtacacaccgcccgtcacaccacgagagttggcaacacccgaagcctgtgagagaaccgtaaggactcagcagtcgaaggtggggctagtaattggggtgaagtcgtaacaaggtagccgtatcggaaggtgcggctggatcacctccttt

SEQ ID NO: 46 - DNA, 1521 bp, Parabacteroides distasonis
cgaagagtttgatcctggctcaggatgaacgctagcgacaggcttaacacatgcaagtcgaggggcagcacaggtagcaataccgggtggcgaccggcgcacgggtgagtaacgcgtatgcaacttgcctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccattactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaaggtagccgtaccggaaggtgcggctggaacacctccttt

(19) United States
(12) Patent Application Publication
(10) Pub. No.: US 2022/0378851 A1
(43) Pub. Date: Dec. 1, 2022

(54) COMPOSITIONS AND METHODS FOR ENHANCING IMMUNE CHECKPOINT INHIBITOR THERAPY

(71) Applicant: Vedanta Biosciences, Inc., Cambridge, MA (US)

(72) Inventors: Rose L. Szabady, Cambridge, MA (US); Bruce Roberts, Framingham, MA (US)

(73) Assignee: Vedanta Biosciences, Inc., Cambridge, MA (US)

(21) Appl. No.: 17/773,863

(22) PCT Filed: Nov. 4, 2020
(86) PCT No.: PCT/US20/58840

§ 371 Date: May 3, 2022

Related U.S. Application Data
(60) Provisional application No. 62/930,456, filed on Nov. 4, 2019.
(60) Provisional application No. 63/014,829, filed on Apr. 24, 2020.

(51) Int. Cl. A61K 35/74; A23L 33/135; C07K 16/28; A61P 35/00

(52) U.S. Cl. CPC A61K 35/74; A23L 33/135; C07K 16/2818; A61P 35/00; A61K 2039/507

(57) ABSTRACT

Provided herein are compositions and methods for treating cancer, treating colitis associated with immune checkpoint inhibitor therapy, colonizing the microbiome, and/or restoring the microbiome, by administering compositions or food products to a subject.

### RELATED APPLICATIONS

This application claims the benefit under 35 U.S.C. § 119(e) of U.S. Provisional Application No. 62/930,456 filed Nov. 4, 2019, and U.S. Provisional Application No. 63/014,829 filed Apr. 24, 2020. The entire contents of each of these referenced applications are incorporated by reference herein in their entirety.

### FIELD

The disclosure relates to compositions and methods for enhancing immune checkpoint inhibitor therapy, treating cancer, treating colitis associated with immune checkpoint inhibitor therapy, colonizing the microbiome, and/or restoring the microbiome, by administering pharmaceutical compositions to a subject.

### BACKGROUND

Humans harbor a population of microbes (collectively referred to as the microbiome) in anatomical locations such as the large intestine, small intestine, mouth, esophagus, stomach, cecum, vagina, skin, nasal cavities, ear, and lungs. The human microbiome is responsible for modulating numerous processes, including digestion and absorption of nutrients, development of the immune system, formation and regeneration of the epithelium, production of metabolites such as bile acids, hormones, vitamins, and short chain fatty acids, and protection from pathogen infections (see, e.g., Mohajeri, et al., Eur J Nutr (2018) 57 (Suppl 1): 1-14). Alterations in the normal microbiota for a human are associated with diseases including cancer, obesity, inflammatory bowel disease, Celiac disease, diabetes mellitus, metabolic syndrome, atherosclerosis, alcoholic liver disease (ALD), nonalcoholic fatty liver disease (NAFLD), and cirrhosis (see, e.g., Wang, et al., Engineering (2017) 3(1): 71-82).

The human gut (large intestine, small intestine, cecum) microbiome regulates carcinogenesis and response to anti-cancer drugs including chemotherapy and immunotherapeutics. The composition of the gut microbiome affects the efficacy and recovery from cancer therapy, although the factors that modulate this response are not understood.

### SUMMARY

Aspects of the present disclosure provide compositions comprising Eubacterium limosum, Subdolinogranulum sp., Phacolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis.

Aspects of the present disclosure provide compositions comprising Fusobacterium ulcerans, Eubacterium limosum, Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions comprising Eubacterium limosum, Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions comprising Fusobacterium ulcerans, Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions comprising Fusobacterium ulcerans, Subdolinogranulum sp., Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions consisting of Eubacterium limosum, Subdolinogranulum sp., Phacolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis.

Aspects of the present disclosure provide compositions consisting of Fusobacterium ulcerans, Eubacterium limosum, Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions consisting of Eubacterium limosum, Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions consisting of Fusobacterium ulcerans, Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions consisting of Fusobacterium ulcerans, Subdolinogranulum sp., Phacolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

Aspects of the present disclosure provide compositions comprising a purified bacterial mixture comprising bacterial strains comprising 16S rDNA sequences of at least 95% sequence identity to the nucleic acid sequences set forth as SEQ ID NOs: 2-11.

Aspects of the present disclosure provide compositions comprising a purified bacterial mixture comprising bacterial strains comprising 16S rDNA sequences of at least 95% sequence identity to the nucleic acid sequences set forth as SEQ ID NOs: 1-8, 10, and 11.

Aspects of the present disclosure provide compositions comprising a purified bacterial mixture comprising bacterial strains comprising 16S rDNA sequences of at least 95% sequence identity to the nucleic acid sequences set forth as SEQ ID NOs: 2-8, 10, and 11.

Aspects of the present disclosure provide compositions comprising a purified bacterial mixture comprising bacterial strains comprising 16S rDNA sequences of at least 95% sequence identity to the nucleic acid sequences set forth as SEQ ID NOs: 1-5, 7, 8, 10, and 11.

Aspects of the present disclosure provide compositions comprising a purified bacterial mixture comprising bacterial strains comprising 16S rDNA sequences of at least 95% sequence identity to the nucleic acid sequences set forth as SEQ ID NOs: 1-5 and 7-11.

Aspects of the present disclosure provide compositions comprising a purified bacterial mixture consisting of bacterial strains comprising 16S rDNA sequences of at least 95% sequence identity to the nucleic acid sequences set forth as SEQ ID NOs: 2-11.

Aspects of the present disclosure provide compositions comprising a purified bacterial mixture consisting of bacterial strains comprising 16S rDNA sequences of at least 95% sequence identity to the nucleic acid sequences set forth as SEQ ID NOs: 1-8, 10, and 11.

Aspects of the present disclosure provide compositions comprising a purified bacterial mixture consisting of bacterial strains comprising 16S rDNA sequences of at least 95% sequence identity to the nucleic acid sequences set forth as SEQ ID NOs: 2-8, 10, and 11.

Aspects of the present disclosure provide compositions comprising a purified bacterial mixture consisting of bacterial strains comprising 16S rDNA sequences of at least 95% sequence identity to the nucleic acid sequences set forth as SEQ ID NOs: 1-5, 7, 8, 10, and 11.

Aspects of the present disclosure provide compositions comprising a purified bacterial mixture consisting of bacterial strains comprising 16S rDNA sequences of at least 95% sequence identity to the nucleic acid sequences set forth as SEQ ID NOs: 1-5 and 7-11.

In some embodiments, the compositions are effective in enhancing checkpoint inhibitor therapy. In some embodiments, the compositions are effective in treating colitis associated with checkpoint inhibitor therapy. In some embodiments, the compositions are effective in inducing the production of regulatory T cells (Tregs) in the intestine. In some embodiments, the compositions are effective in inducing the production of IFNγ+ CD8+ T cells in the intestine.

In some embodiments, the bacterial strains are lyophilized. In some embodiments, the bacterial strains are spray-dried. In some embodiments, one or more of the bacterial strains are in spore form. In some embodiments, each of the bacterial strains is in spore form. In some embodiments, one or more of the bacterial strains are in vegetative form. In some embodiments, each of the bacterial strains are in vegetative form.

In some embodiments, the compositions further comprise one or more anticancer agents. In some embodiments, the anticancer agent is a chemotherapy agent. In some embodiments, the anticancer agent is a cancer immunotherapy agent. In some embodiments, the cancer immunotherapy agent is an immune checkpoint inhibitor.

In some embodiments, the immune checkpoint inhibitor is a PD-1 inhibitor, PD-L-1 inhibitor, or CTLA-4 inhibitor. In some embodiments, the immune checkpoint inhibitor is a PD-1 inhibitor. In some embodiments, the PD-1 inhibitor is nivolumab. In some embodiments, the PD-1 inhibitor is pembrolizumab. In some embodiments, the PD-1 inhibitor is pidiluzimab. In some embodiments, the immune checkpoint inhibitor is a PD-L-1 inhibitor. In some embodiments, the PD-L-1 inhibitor is avelumab. In some embodiments, the PD-L-1 inhibitor is durvalumab. In some embodiments, the immune checkpoint inhibitor is a CTLA-4 inhibitor. In some embodiments, the CTLA-4 inhibitor is an anti-CTLA-4 antibody. In some embodiments, the CTLA-4 inhibitor is ipilimumab. In some embodiments, the CTLA-4 inhibitor is tremelimumab.

Aspects of the present disclosure provide pharmaceutical compositions comprising any of the compositions described herein, further comprising a pharmaceutically acceptable excipient. Aspects of the present disclosure provide food products comprising any of the compositions described herein. In some embodiments, the pharmaceutical compositions are formulated for oral delivery. In some embodiments, the pharmaceutical compositions are formulated for rectal delivery. In some embodiments, the pharmaceutical compositions are formulated for delivery to the intestine. In some embodiments, the pharmaceutical compositions are formulated for delivery to the colon.

In some embodiments, the pharmaceutical compositions are administered as one dose. In some embodiments, the pharmaceutical compositions are administered as multiple doses. In some embodiments, each dose comprises the administration of multiple capsules.

Aspects of the present disclosure provide methods for treating cancer, comprising administering to the subject any of the compositions or food products described herein in an effective amount to treat the cancer. In some embodiments, the cancer is cancer is carcinoma, glioma, mesothelioma, melanoma, lymphoma, leukemia, adenocarcinoma, breast cancer, ovarian cancer, cervical cancer, glioblastoma, multiple myeloma, prostate cancer, Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumors, or rhabdomyosarcoma. In some embodiments, the cancer is prostate cancer, bladder cancer, non-small cell lung cancer, urothelial carcinoma, melanoma, or renal cell carcinoma.

In some embodiments, an antibiotic is administered prior to the administration of any one of the compositions. In some embodiments, the subject is human. In some embodiments, the composition is administered to the subject more than once. In some embodiments, the composition is administered to the subject by oral administration. In some embodiments, the composition is administered to the subject by rectal administration.

In some embodiments, the methods further comprise administering an antibiotic to the subject prior to administration of any one of the compositions or food products. In some embodiments, any of the compositions or food products is administered to the subject in multiple doses. In some embodiments, if one or more bacterial strains of the compositions colonize the subject to a level above a threshold level, no further compositions or food products are administered to the subject. In some embodiments, if the compositions induce a CD8+ T cell response above a threshold level, no further compositions or food products are administered to the subject.

Aspects of the present disclosure provide methods of treating colitis associated with checkpoint inhibitor therapy in a subject, comprising administering to the subject a therapeutically effective amount of any of the compositions or food products described herein.

In some embodiments, an antibiotic is administered prior to the administration of any one of the compositions. In some embodiments, the subject is human. In some embodiments, the composition is administered to the subject more than once. In some embodiments, the composition is administered to the subject by oral administration. In some embodiments, the composition is administered to the subject by rectal administration.

In some embodiments, the methods further comprise administering an antibiotic to the subject prior to administration of any one of the compositions or food products. In some embodiments, any of the compositions or food products is administered to the subject in multiple doses. In some embodiments, if one or more bacterial strains of the compositions colonize the subject to a level above a threshold level, no further compositions or food products are administered to the subject. In some embodiments, if the compositions induce a CD8+ T cell response above a threshold level, no further compositions or food products are administered to the subject.

Aspects of the present disclosure provide methods for colonizing the microbiome of a subject, the methods comprising administering to the subject any of the compositions or food products described herein in an effective amount to colonize the microbiome.

In some embodiments, an antibiotic is administered prior to the administration of any one of the compositions. In some embodiments, the subject is human. In some embodiments, the composition is administered to the subject more than once. In some embodiments, the composition is administered to the subject by oral administration. In some embodiments, the composition is administered to the subject by rectal administration.

In some embodiments, the methods further comprise administering an antibiotic to the subject prior to administration of any one of the compositions or food products. In some embodiments, any of the compositions or food products is administered to the subject in multiple doses. In some embodiments, if one or more bacterial strains of the compositions colonize the subject to a level above a threshold level, no further compositions or food products are administered to the subject. In some embodiments, if the compositions induce a CD8+ T cell response above a threshold level, no further compositions or food products are administered to the subject.

Aspects of the present disclosure provide methods for restoring a microbiome in a subject, the methods comprising administering to the subject any of the compositions or food products described herein in an effective amount to restore the microbiome in the subject. In some embodiments, the subject has not undergone a dysbiosis inducing event. In some embodiments, the subject has not had an infectious disease. In some embodiments, the subject has not been treated with an antibiotic.

In some embodiments, the subject has undergone a dysbiosis inducing event. In some embodiments, the dysbiosis inducing event is treatment with one or more antibiotics. In some embodiments, the subject is human. In some embodiments, the composition is administered to the subject more than once. In some embodiments, the composition is administered to the subject by oral administration. In some embodiments, the composition is administered to the subject by rectal administration.

In some embodiments, the methods further comprise administering an antibiotic to the subject prior to administration of any one of the compositions or food products. In some embodiments, any of the compositions or food products is administered to the subject in multiple doses. In some embodiments, if one or more bacterial strains of the compositions colonize the subject to a level above a threshold level, no further compositions or food products are administered to the subject. In some embodiments, if the compositions induce a CD8+ T cell response above a threshold level, no further compositions or food products are administered to the subject.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIG. 1 shows a plot depicting the results of a whole genome shotgun sequencing Principal Component Analysis (PCA) of the gut microbiome from melanoma patients that responded to immune checkpoint inhibitor therapy (Responder, dark gray data points) versus those that did not respond (Non-Responder, light gray data points). The beta diversity (Bray-Curtis) was significantly different between the Responders and the Non-Responders.

FIG. 2 presents a chart showing results of a sparse logistic regression analysis to identify taxa of gut microbiome bacteria from melanoma patients that responded to immune checkpoint inhibitor therapy (ICT) compared to patients that did not respond to ICT. The importance, measured as the model selection frequency for differentiating features in 100 random subsets of data, is shown for each of the indicated bacterial strains selected at an 80% importance threshold.

FIG. 3 shows an example model of colonization of bacterial composition VE800 and induction of IFNγ (IFNg+) production and anti-tumor activity.

FIGS. 4A-4C show an experimental protocol for examining immune responses in germ-free mice following administration of purified bacterial compositions. FIG. 4A shows a schematic of the experimental protocol in which germ-free C57BL/6J mice were treated with vehicle, purified bacterial compositions (LBP: “live bacterial product”), or stool fraction library (SFL). Treated mice are sacrificed after 3 weeks (D21) and lamina propria lymphocytes (LPLs) are isolated to quantify IFNg+ CD8 T cells or after 4 weeks (D28) and lamina propria lymphocytes (LPLs) are isolated to quantify regulatory T cells (Tregs). FIGS. 4B and 4C present data from two independent experiments in separate facilities showing the frequency of CD8+ IFNγ+ and frequency of CD8+ IFNγ+ LPLs, respectively, following treatment with vehicle (Germ-free), VE800, VE802, VE803, or VE804 bacterial compositions.

FIG. 5 presents a graph depicting induction of CD8+ IFNγ cells in control mice (Germ-free) or mice administered the indicated bacterial compositions: VE800, VE804, VE805, or VE806.

FIG. 6 shows a schematic of a B16 melanoma model for assessing the effects of administration of purified bacterial compositions. C57BL/6 mice were administered antibiotics (Penicillin G+Streptomycin) for 6 days. On day 0 (DO), melanoma cells (B16F10) were injected subcutaneously in both flanks. Immune checkpoint inhibitor therapy (“ICT”) involving an anti-PD-1 antibody or the combination of an anti-PD-1 antibody and an anti-CTLA-4 antibody were administered on days 3, 6, and 9. Administration of purified bacterial compositions (LBP: “live bacterial products”) VE800, VE804, or VE411 (control) were administered 3 times a week for 3 weeks. M is Monday, W is Wednesday, F is Friday.

FIGS. 7A-7E present graphs showing results of the B16 melanoma model depicted in FIG. 6. FIGS. 7A-7D show tumor volume in the indicated treatment groups. “Antibiotic” refers to antibiotic pre-treatment prior to melanoma cell injection, as shown in FIG. 6; “continuous antibiotic” refers to continuous antibiotic treatment through the course of the experiment. FIGS. 7C and 7D correspond to different flank sides of the same mice. FIG. 7E shows survival of mice in FIGS. 7C and 7D. For FIGS. 7A and 7B, ICT is immune checkpoint therapy using a combination of anti-PD1 antibodies and anti-CTLA4 antibodies. For FIGS. 7C-7E, ICT is immune checkpoint therapy using anti-PD-L-1 antibodies or a combination of anti-PD1 antibodies and anti-CTLA4 antibodies.

FIG. 8 presents a graph depicting effector regulatory T cell (Treg) induction (percent Foxp3+Rorgt+ in CD4+ T cells) in control mice (germ-free, “GF”), mice administered the VE800 bacterial composition, or specific-pathogen free (“SPF”) mice. “Rorgt” is RAR-related orphan receptor gamma.

FIGS. 9A and 9B present graphs depicting induction of regulatory T cells in the B16 melanoma model. FIG. 9A shows total regulatory T cells (Tregs; percent CD4+ Foxp3+) in control mice (germ-free, “GF”) or mice administered the indicated bacterial compositions: VE800, VE802, VE803, or VE804. FIG. 9B shows effector regulatory T cells (effector Tregs; percent CD4+ Foxp3+RORγt+) in control mice (germ-free, “GF”) or mice administered the indicated bacterial compositions: VE800, VE802, VE803, or VE804. RORγt is RAR-related orphan receptor gamma.

FIGS. 10A and 10B present graphs depicting induction of regulatory T cells in the B16 melanoma model. FIG. 10A shows total regulatory T cells (Tregs frequency CD4⁺ Foxp3⁺) in control mice (germ-free, “GF”) or mice administered the indicated bacterial compositions: VE800, VE802, VE803, VE804, or VE805. FIG. 10B shows induced/peripheral regulatory T cells (Tregs; percent CD4+ Foxp3+Helios^{neg})

FIGS. 11A and 11B present an experimental protocol for examining the induction of IFNγ in CD8⁺ T cells following administration of purified bacterial compositions. FIG. 11A shows a schematic of the experimental protocol in which germ-free mice were administered bacterial composition VE800 or VE804. IFNγ induction in lamina propria lymphocyte (LPLs) was assessed 3 weeks after treatment. FIG. 11B shows the normalized frequency of IFNγ+ CD8⁺ T cells in control mice (Germ-free) and mice administered bacterial composition VE800 or bacterial composition VE804. The data in FIG. 11B are combined from individual experiments.

FIG. 12 shows a schematic of a B16 melanoma model for assessing effects of administration of purified bacterial compositions. Specific pathogen free (SPF) C57BL/6 mice were administered antibiotics (Penicillin G+Streptomycin) for 6 days. On day 0 (DO), melanoma cells (B16F10) were injected subcutaneously. Immune checkpoint inhibitors (a combination of anti-PD-1 antibodies and anti-CTLA-4 antibodies) were administered on days 4, 8, and 12 following injection with melanoma cells. Bacterial compositions are administered 3 times a week for 3 weeks following injection with melanoma cells vial oral gavage (“consortia administration”). ICT is immune checkpoint therapy.

FIGS. 13A-13D present graphs showing results of the B16 melanoma model depicted in FIG. 12. FIGS. 13A and 13B tumor volume in the indicated treatment groups at the indicated time points. FIGS. 13C and 13D show survival of mice in the indicated treatment groups at the indicated time points.

FIGS. 14A and 14B present graphs showing results of the B16 melanoma model depicted in FIG. 12. FIG. 14A shows tumor volume in the indicated treatment groups at the indicated time points. FIG. 14B shows survival of mice in the indicated treatment groups at the indicated time points.

FIGS. 15A-15C present an experimental protocol for examining the endogenous microbiome recovery after antibiotic treatment and administration of purified bacterial compositions. FIG. 15A shows a schematic of the experimental protocol in which specific pathogen free (SPF) mice were administered vancomycin and then treated with vehicle or bacterial composition VE800. Whole genome sequencing was used to examine endogenous microbiome recovery and colonization by VE800 bacterial strains at various time points. FIG. 15B shows the relative abundance of all phyla prior to antibiotic treatment (“Pre Abx”), following antibiotic treatment (“Post Abx”), at VE800 dosing, day 3 post-VE800 dosing, and day 21 post-VE800 dosing. FIG. 15C shows the absolute abundance of each bacterial strain of composition VE800 prior to antibiotic treatment (“Pre Abx”), following antibiotic treatment (“Post Abx”), at VE800 dosing, day 3 post-VE800 dosing, and day 21 post-VE800 dosing.

FIGS. 16A-16E show that colonization dynamics of bacterial composition VE800 are associated with IFNγ induction of colonic CD8+ T cells. Specific pathogen free (SPF) mice were administered antibiotics followed by treatment with bacterial composition VE800. IFNγ induction in colonic CD8+ T cells and bacterial colonization were assessed at 3 weeks post-treatment. FIG. 16A shows normalized frequency of IFNγ+ CD8+ T cells following treatment with vehicle control, 1 dose of 10e8 CFU VE800, or 3 consecutive daily doses of 10e8 CFU VE800. FIG. 16B shows normalized frequency of IFNγ+ CD8+ T cells following treatment with vehicle control, 3 consecutive daily doses of 10e8 CFU VE800, or 3 doses of 10e9 CFU VE800. FIG. 16C shows the relative abundance of VE800 strains following treatment with vehicle control, 1 dose of 10e8 CFU VE800, or 3 consecutive daily doses of 10e8 CFU VE800. FIG. 16D shows the summed relative abundance of VE800 strains following treatment with vehicle control, 3 consecutive daily doses of 10e8 CFU VE800, or 3 consecutive daily doses of 10e9 CFU VE800. FIG. 16E shows a plot depicting regression analysis of the normalized frequency of IFNγ+ CD8+ T cells relative to the sum of VE800 strains (relative abundance). CFU is colony forming units.

FIGS. 17A and 17B show that colonization dynamics of bacterial composition VE800 are associated with IFNγ induction of colonic CD8+ T cells. FIG. 17A shows a schematic of the experimental protocol in which SPF mice were administered vancomycin (“Vanco”) and then treated with vehicle or bacterial composition VE800. FIG. 17B shows the percent IFNγ+ in CD8+ T cells following treatment with vehicle control, 3 doses of 10e7 CFU VE800, or 3 doses of 10e9 CFU VE800. * indicates time points at which samples were obtained for analysis.

FIGS. 18A and 18B show that higher CFU density in the initial bacterial dose led to an increase in the sustained induction of IFNγ in CD8+ T cells. FIG. 18A shows an experimental protocol for examining the induction of IFNγ in CD8+ T cells at both a shorter and longer term timepoints following administration of purified bacterial compositions. Specific pathogen free (SPF) mice were administered antibiotics (e.g., vancomycin) followed by treatment with bacterial composition VE800. IFNγ induced in colonic CD8+ T cells and bacterial colonization were assessed at 3 and 8 weeks post-treatment. FIG. 18B shows frequency of IFNγ+ CD8+ T cells following treatment with vehicle control, 3 consecutive daily doses of 10e8 CFU VE800, or 3 consecutive daily doses of 10e9 CFU VE800 and measured at 3 or 8 weeks following initial dosing. “d21” is day 21 after first dose and “d56” is day 56 after first dose.

FIG. 19 shows the frequency of IFNγ+ CD8+ T cells in germ-free mice administered a mixture of 21 live bacterial strains (“21-mix”), a mixture of 11 live bacterial strains (“11-mix”), a mixture of 10 live bacterial strains (“10-mix”), or control (see, Tanoue et al., Nature, 565(7741): 600-605). The 21-mix is a mixture of the 11-mix and the 10-mix, the 11-mix is VE800, the 10-mix is the inactive fraction of the 21-mix. GF is germ-free, and NS is not significant. *, **, or *** above a line indicate a statistically significant difference between the groups specified by the endpoints of the line.

FIGS. 20A and 20B show that the 11-mix increases CD8+ tumor-infiltrating lymphocytes (TIL) and improves the efficacy of checkpoint therapy in specific pathogen free (SPF) mice injected with a murine tumor model (MC38, Braf^{V600E}Pten^{-/-}). FIG. 20A shows tumor volume in SPF mice administered anti-PD-1 inhibitor, the 10-mix, the combination of 10-mix+anti-PD-1 inhibitor, the 11-mix (VE800), or the combination of 11-mix+anti-PD-1 inhibitor. FIG. 20B shows the frequency of IFNγ+ CD8+ T cells TILs in SPF mice administrated the anti-PD-1 inhibitor, the 11-mix (VE800), the combination of 11-mix+anti-PD-1, the 10-mix, or the combination of 10-mix+anti-PD-1. PD-1 is programmed cell death protein 1. See, Tanoue et al., Nature (2019) 565(7741): 600-605.

FIG. 21 shows a Simon 2-stage study design for assessing the safety, tolerability, and clinical activity of VE800 (11-mix) in combination with the anti-PD-1 inhibitor nivolumab (Opdivo®) in 3 patient cohorts. Cohort A includes melanoma patients, Cohort B includes patients with gastric/gastroesophageal junction (GEJ) adenocarcinoma, and Cohort C includes patients with microsatellite-stable colorectal cancer (CRC-MSS). ORR is objective response rate.

FIG. 22 shows a schematic of the study for assessing VE800 in combination with the anti-PD-1 inhibitor. Patients are screened from day −21 to day −5, then orally administered vancomycin for 5 days (days −4 to day 0). The treatment period begins on day 1 and involves administration of VE800 daily (QD) and administration of the anti-PD-1 inhibitor (nivolumab) every 4 weeks (Q4W). Survival is assessed by follow up every 90 days (Q90).

### DETAILED DESCRIPTION

Although immune checkpoint inhibitor therapies have been effective in treating cancer by targeting downregulators of the anticancer immune response, treatment with immune checkpoint inhibitors can be associated with adverse effects, such as colitis, likely due to the inflammatory T cell activation. See, Som et al. World J. Clin. Cases. (2019) 7(4): 405-418. Provided herein are compositions and methods for enhancing efficacy of immune checkpoint inhibitor therapy. Provided herein are compositions and methods for treating cancer and/or treating colitis associated with an immune checkpoint inhibitor therapy. Provided herein are compositions and methods for colonizing a microbiome and/or restoring a microbiome of a subject. In some embodiments, the bacterial compositions described herein induce regulatory T cells and/or IFNγ+ CD8+ T cells. In some embodiments, the bacterial compositions described herein have the ability to induce regulatory T cells (e.g., to treat colitis), while also being effective in anticancer therapy by inducing IFNγ+ CD8+ T cells (e.g., in combination with an immune-checkpoint inhibitor).

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the present disclosure provide compositions comprising mixtures of bacterial strains. Some embodiments of the bacterial strains are described for instance in PCT Publication Nos. WO 2018/117263, WO2019/156234, and Tanoue et al. Nature (2019) 565: 600-605, which are incorporated herein by reference in their entirety.

In some embodiments, the composition comprises a purified bacterial mixture comprising Eubacterium limosum, Subdolinogranulum sp., Phascolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis. In some embodiments, the composition comprises a purified bacterial mixture consisting of Eubacterium limosum, Subdolinogranulum sp., Phascolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis. In some embodiments, the composition comprises a purified bacterial mixture consisting essentially of Eubacterium limosum, Subdolinogranulum sp., Phascolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis.

In some embodiments, the composition comprises a purified bacterial mixture comprising Fusobacterium ulcerans, Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting of Fusobacterium ulcerans, Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting essentially of Fusobacterium ulcerans, Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

In some embodiments, the composition comprises a purified bacterial mixture comprising Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting of Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting essentially of Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

In some embodiments, the composition comprises a purified bacterial mixture comprising Fusobacterium ulcerans, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting of Fusobacterium ulcerans, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting essentially of Fusobacterium ulcerans, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

In some embodiments, the composition comprises a purified bacterial mixture comprising Fusobacterium ulcerans, Subdolinogranulum sp., Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting of Fusobacterium ulcerans, Subdolinogranulum sp., Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis. In some embodiments, the composition comprises a purified bacterial mixture consisting essentially of Fusobacterium ulcerans, Subdolinogranulum sp., Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

The disclosure also encompasses compositions comprising bacterial strains that are close in homology to and/or fall within the species Fusobacterium ulcerans, Parabacteroides johnsonii, Paraprevotella xylaniphila, Parabacteroides distasonis, Alistipes sp., Eubacterium limosum, Bacteroides dorei, Parabacteroides gordonii, Subdolinogranulum sp., Bacteroides uniformis, and Phascolarctobacterium faecium.

In some embodiments, the purified bacterial mixture does not comprise Fusobacterium ulcerans. In some embodiments, the purified bacterial mixture does not comprise Subdolinogranulum sp. In some embodiments, the purified bacterial mixture does not comprise Eubacterium limosum. In some embodiments, the purified bacterial mixture does not comprise Fusobacterium ulcerans or Subdolinogranulum sp. In some embodiments, the purified bacterial mixture does not comprise Subdolinogranulum sp. or Eubacterium limosum.

In some embodiments, the composition comprises a purified bacterial mixture comprising Fusobacterium ulcerans. In some embodiments, the composition comprises a purified bacterial mixture comprising Fusobacterium ulcerans and one or more bacterial strains selected from the group consisting of Eubacterium limosum, Subdolinogranulum sp., Phascolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis.

In one aspect, the disclosure provides compositions comprising purified bacterial strains related to the following species Clostridiales bacterium sp., Bacteroides fragilis, Bacteroides caccae, Fusicatenibacter saccharivorans, Roseburia intestinalis CAG:13, Eubacterium sp. CAG:180, Firmicutes bacterium CAG:56, Bacteroides uniformis, Bacteroides thetaiotaomicron CAG:40, Bacteroides intestinalis, Bacteroides massiliensis, Akkermansia muciniphila, Parabacteriodes distasonis, Bacteroides vulgatus, Clostridiales bacterium 52_15, and Bacteroides plebelus. In some embodiments, the disclosure provides compositions comprising one or more (e.g., 1, 2, 3, 4, 5, 6, 7, 8, 9, 10, 11, 12, 13, 14, 15, or 16) purified bacterial strain of species selected from the group consisting of Clostridiales bacterium sp., Bacteroides fragilis, Bacteroides caccae, Fusicatenibacter saccharivorans, Roseburia intestinalis CAG:13, Eubacterium sp. CAG:180, Firmicutes bacterium CAG:56, Bacteroides uniformis, Bacteroides thetaiotaomicron CAG:40, Bacteroides intestinalis, Bacteroides massiliensis, Akkermansia muciniphila, Parabacteriodes distasonis, Bacteroides vulgatus, Clostridiales bacterium 52_15, and Bacteroides plebelus. In some embodiments, the disclosure provides compositions consisting of one or more (e.g., 1, 2, 3, 4, 5, 6, 7, 8, 9, 10, 11, 12, 13, 14, 15, or 16) purified bacterial strain of species selected from the group consisting of Clostridiales bacterium sp., Bacteroides fragilis, Bacteroides caccae, Fusicatenibacter saccharivorans, Roseburia intestinalis CAG:13, Eubacterium sp. CAG:180, Firmicutes bacterium CAG:56, Bacteroides uniformis, Bacteroides thetaiotaomicron CAG:40, Bacteroides intestinalis, Bacteroides massiliensis, Akkermansia muciniphila, Parabacteriodes distasonis, Bacteroides vulgatus, Clostridiales bacterium 52_15, and Bacteroides plebelus. In some embodiments, the disclosure provides compositions consisting essentially of one or more (e.g., 1, 2, 3, 4, 5, 6, 7, 8, 9, 10, 11, 12, 13, 14, 15, or 16) purified bacterial strain of species selected from the group consisting of Clostridiales bacterium sp., Bacteroides fragilis, Bacteroides caccae, Fusicatenibacter saccharivorans, Roseburia intestinalis CAG:13, Eubacterium sp. CAG:180, Firmicutes bacterium CAG:56, Bacteroides uniformis, Bacteroides thetaiotaomicron CAG:40, Bacteroides intestinalis, Bacteroides massiliensis, Akkermansia muciniphila, Parabacteriodes distasonis, Bacteroides vulgatus, Clostridiales bacterium 52_15, and Bacteroides plebelus.

In some embodiments, the compositions described herein comprise at least 2, at least 3, at least 4, at least 5, at least 6, at least 7, at least 8, at least 9, at least 10, at least 11, at least 12, at least 13, at least 14, at least 15, or at least 16 or more bacterial strains (e.g., purified bacterial strains).

Aspects of the present disclosure provide bacterial strains with 16S rDNA sequences that have sequence identity to a nucleic acid sequence of any one of the sequences of the bacterial strains or species described herein. As will be appreciated by one of ordinary skill in the art, the 16S rDNA sequences represent DNA sequences corresponding to the 16S rRNA sequences. The terms “identical” or “percent identity,” in the context of two or more nucleic acid sequences or amino acid sequences, refers to two or more sequence or subsequences that are the same. Two sequences are “substantially identical” if two sequences have a specified percentage of amino acid residues or nucleotides that are the same (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity) over a specified region or over the entire sequence, when compared and aligned for maximum correspondence over a comparison window or designated region as measured using a sequence comparison algorithm or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length of the 16S rDNA or 16S rDNA sequence.

In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity relative to any of the strains or bacterial species described herein over a specified region or over the entire sequence. It would be appreciated by one of skill in the art that the term “sequence identity” or “percent sequence identity” in the context of two or more nucleic acid sequences or amino acid sequences, refers to a measure of similarity between two or more sequences or portion(s) thereof.

In one aspect, the bacteria of the compositions disclosed herein can be identified by their 16S rRNA (or 16S rDNA) nucleic acid sequence. In general, bacteria are classified as belonging to a specific species and/or genera based on their 16S rRNA nucleic acid sequence. Bacteria, such as bacteria derived from the microbiome, may also be classified into phylogenetic clusters with other closely related strains and species. (See e.g., Rajilic-Stojanovic, M., and de Vos, W. M. (2014). The first 1000 cultured species of the human gastrointestinal microbiota. FEMS Microbiol Rev 38, 996-1047). Methods for determining the identity of specific bacterial species based on their 16S rRNA (or 16S rDNA) nucleic acid sequences are well known in the art (See, e.g., Jumpstart Consortium Human Microbiome Project Data Generation Working, G. (2012). Evaluation of 16S rDNA-based community profiling for human microbiome research. PLoS One 7, e39315).

Aspects of the present disclosure provide compositions comprising a purified bacterial mixture comprising bacterial strains comprising 16S rDNA sequences of at least 97% sequence identity to the nucleic acid sequences set forth as SEQ ID NOs: 1 and 3-11. Aspects of the present disclosure provide compositions comprising a purified bacterial mixture consisting of bacterial strains comprising 16S rDNA sequences of at least 97% sequence identity to the nucleic acid sequences set forth as SEQ ID NOs: 1 and 3-11. Aspects of the present disclosure provide compositions comprising a purified bacterial mixture consisting essentially of bacterial strains comprising 16S rDNA sequences of at least 97% sequence identity to the nucleic acid sequences set forth as SEQ ID NOs: 1 and 3-11.

Aspects of the present disclosure provide compositions comprising a purified bacterial mixture comprising bacterial strains comprising 16S rDNA sequences of at least 97% sequence identity to the nucleic acid sequences set forth as SEQ ID NOs: 1-4 and 6-11. Aspects of the present disclosure provide compositions comprising a purified bacterial mixture consisting of bacterial strains comprising 16S rDNA sequences of at least 97% sequence identity to the nucleic acid sequences set forth as SEQ ID NOs: 1-4 and 6-11. Aspects of the present disclosure provide compositions comprising a purified bacterial mixture consisting essentially of bacterial strains comprising 16S rDNA sequences of at least 97% sequence identity to the nucleic acid sequences set forth as SEQ ID NOs: 1-4 and 6-11.

Aspects of the present disclosure provide compositions comprising a purified bacterial mixture comprising bacterial strains comprising 16S rDNA sequences of at least 97% sequence identity to the nucleic acid sequences set forth as SEQ ID NOs: 1, 3, 4, and 6-11. Aspects of the present disclosure provide compositions comprising a purified bacterial mixture consisting of bacterial strains comprising 16S rDNA sequences of at least 97% sequence identity to the nucleic acid sequences set forth as SEQ ID NOs: 1, 3, 4, and 6-11. Aspects of the present disclosure provide compositions comprising a purified bacterial mixture consisting essentially of bacterial strains comprising 16S rDNA sequences of at least 97% sequence identity to the nucleic acid sequences set forth as SEQ ID NOs: 1, 3, 4, and 6-11.

Aspects of the present disclosure provide compositions comprising a purified bacterial mixture consisting of bacterial strains comprising 16S rDNA sequences of at least 97% sequence identity to the nucleic acid sequences set forth as SEQ ID NOs: 1-4, 6-9, and 11. Aspects of the present disclosure provide compositions comprising a purified bacterial mixture consisting of bacterial strains comprising 16S rDNA sequences of at least 97% sequence identity to the nucleic acid sequences set forth as SEQ ID NOs: 1-4, 6-9, and 11. Aspects of the present disclosure provide compositions comprising a purified bacterial mixture consisting essentially of bacterial strains comprising 16S rDNA sequences of at least 97% sequence identity to the nucleic acid sequences set forth as SEQ ID NOs: 1-4, 6-9, and 11.

Aspects of the present disclosure provide compositions comprising a purified bacterial mixture comprising bacterial strains comprising 16S rDNA sequences of at least 97% sequence identity to the nucleic acid sequences set forth as SEQ ID NOs: 1-9 and 11. Aspects of the present disclosure provide compositions comprising a purified bacterial mixture consisting of bacterial strains comprising 16S rDNA sequences of at least 97% sequence identity to the nucleic acid sequences set forth as SEQ ID NOs: 1-9 and 11. Aspects of the present disclosure provide compositions comprising a purified bacterial mixture consisting essentially of bacterial strains comprising 16S rDNA sequences of at least 97% sequence identity to the nucleic acid sequences set forth as SEQ ID NOs: 1-9 and 11.

In one aspect, the disclosure provides compositions comprising multiple purified bacterial strains. In one aspect, the 16S rDNA sequences of purified bacterial strains of the compositions were compared to 16S rDNA sequences of known bacterial species/strains in a bacterial genome database to identify the closest known related bacterial species to the bacterial strains disclosed herein (See, e.g., Table 1). It should be appreciated that multiple bacterial strains of the compositions disclosed herein may have the same closest related bacterial species.

In one aspect, the disclosure provides bacterial strains and combinations of bacterial strains that are homologous or have a high percent of homology with bacterial strains comprising 16S rDNA sequences provided by SEQ ID NOs:1-11. As discussed previously, in some embodiments, the bacterial strains are purified. The bacterial strains disclosed herein that have a 16S rDNA sequence with a nucleic acid sequence provided by SEQ ID NOs:1-11 have a high percent of homology (e.g., greater than 90%), or sequence identity, with 16S rDNA sequences of bacterial strains that have been described in various databases (See, e.g., the National Center for Biotechnology Information). Table 1 provides the closest known species by homology when the 16S rDNA sequences comprising SEQ ID NOs:1-11 are compared to 16S rDNA sequences of bacterial species available in public databases. Throughout the instant application the bacterial strains associated with 16S rDNA sequences comprising SEQ ID NOs: 1-11 may alternatively or in addition be referred to by any of the additional nucleic acid sequences provided by SEQ ID NOs: 36-46, as shown in Table 1.

By way of example, the bacterial strain comprising a 16S rDNA sequence with SEQ ID NO:1 disclosed herein has the highest homology with a bacterial strain of the species Phascolarctobacterium faecium as defined by NCBI Accession #LN998073, as shown in Table 1. While the bacterial strain with SEQ ID NO:1 has homology with other published bacterial strains as well, the highest homology is with a bacterial strain of the species Phascolarctobacterium faecium as defined by NCBI Accession #LN998073. It should be appreciated that multiple bacterial strains disclosed herein may have the highest homology with the same species.

As will be appreciated by one of skill in the art, the bacterial strains disclosed herein may identified as having a high level of homology with a bacterial species but may be referred to by an alternative species name. It should further be appreciated that the bacterial strains disclosed herein that have a 16S rDNA sequence with a nucleic acid sequence selected from the group consisting of SEQ ID NOs:1-11 are also homologous to other strains based on their whole genome sequence, or subset of their whole genome sequence. For example, the bacterial strain comprising a 16S rDNA sequence with SEQ ID NO:1 disclosed herein has the highest homology with a bacterial strain of the species Phascolarctobacterium faecium, whereas based on whole genome sequence, the species with the highest homology is Phascolarctobacterium sp. CAG:207, as shown in Table 1. The bacterial strain comprising a 16S rDNA sequence of SEQ ID NO: 1 may be referred to as a bacterial strain of the species Phascolarctobacterium faecium but, in addition or alternatively, may be referred to as Phascolarctobacterium sp. CAG:207. The bacterial strain comprising a 16S rDNA sequence of SEQ ID NO: 1 may be referred to by the 16S rDNA sequence of SEQ ID NO: 36.

The bacterial strain comprising a 16S rDNA sequence of SEQ ID NO: 2 disclosed herein has the highest homology with a bacterial strain of the species Fusobacterium varium, whereas based on whole genome sequence, the species with the highest homology is Fusobacterium ulcerans. The bacterial strain comprising a 16S rDNA sequence of SEQ ID NO: 2 may be referred to as a bacterial strain of the species Fusobacterium varium but, in addition or alternatively, may be referred to as Fusobacterium ulcerans, Fusobacterium sp. or Fusobacterium A sp. The bacterial strain comprising a 16S rDNA sequence of SEQ ID NO: 2 may be referred to by the 16S rDNA sequence of SEQ ID NO: 37.

The bacterial strain comprising a 16S rDNA sequence of SEQ ID NO: 3 disclosed herein has the highest homology with a bacterial strain of the species Bacteroides dorei. The bacterial strain comprising a 16S rDNA sequence of SEQ ID NO: 3 may be referred to as a bacterial strain of the species Bacteroides dorei but, in addition or alternatively, may be referred to as Bacteroides fluxus, Phoccaeicola dorei, or Bacteroides_B dorei. The bacterial strain comprising a 16S rDNA sequence of SEQ ID NO: 3 may be referred to by the 16S rDNA sequence of SEQ ID NO: 38.

The bacterial strain comprising a 16S rDNA sequence of SEQ ID NO: 4 disclosed herein has the highest homology with a bacterial strain of the species Bacteroides uniformis, whereas based on whole genome sequence, the species with the highest homology is Bacteroides sp. D20. The bacterial strain comprising a 16S rDNA sequence of SEQ ID NO: 4 may be referred to as a bacterial strain of the species Bacteroides uniformis but, in addition or alternatively, may be referred to as Bacteroides sp. 20. The bacterial strain comprising a 16S rDNA sequence of SEQ ID NO: 4 may be referred to by the 16S rDNA sequence of SEQ ID NO: 39.

The bacterial strain comprising a 16S rDNA sequence of SEQ ID NO: 5 disclosed herein has the highest homology with a bacterial strain of the species Subdolingogranulum sp. or Gemminger formicilis, whereas based on whole genome sequence, the species with the highest homology is Ruminococcaceae bacterium cv2 The bacterial strain comprising a 16S rDNA sequence of SEQ ID NO: 5 may be referred to as a bacterial strain of the species Gemminger formicilis, but, in addition or alternatively, may be referred to as Subdolingogranulum sp., Ruminococcaceae bacterium cv2, Ruminococcaeae bacterium, or Ruthenibacterium lactatiformans. The bacterial strain comprising a 16S rDNA sequence of SEQ ID NO: 5 may be referred to by the 16S rDNA sequence of SEQ ID NO: 40.

The bacterial strain comprising a 16S rDNA sequence of SEQ ID NO: 6 may be referred to by the 16S rDNA sequence of SEQ ID NO: 41.

The bacterial strain comprising a 16S rDNA sequence of SEQ ID NO: 7 may be referred to by the 16S rDNA sequence of SEQ ID NO: 42.

The bacterial strain comprising a 16S rDNA sequence of SEQ ID NO: 8 disclosed herein has the highest homology with a bacterial strain of the species Alistipes sp. or Alistipes senegalensis. The bacterial strain comprising a 16S rDNA sequence of SEQ ID NO: 8 may be referred to as a bacterial strain of the species Alistipes senegalensis, but, in addition or alternatively, may be referred to as Alistipes sp. or Alistipes timonensis. The bacterial strain comprising a 16S rDNA sequence of SEQ ID NO: 8 may be referred to by the 16S rDNA sequence of SEQ ID NO: 43.

The bacterial strain comprising a 16S rDNA sequence of SEQ ID NO: 9 disclosed herein has the highest homology with a bacterial strain of the species Parabacteroides gordonii, whereas based on whole genome sequence, the species with the highest homology is Parabacteroides sp. HGS0025. The bacterial strain comprising a 16S rDNA sequence of SEQ ID NO: 9 may be referred to as a bacterial strain of the species Parabacteroides gordonii, but, in addition or alternatively, may be referred to as Parabacteroides sp. HGS0025 or Parabacteroides timonensis. The bacterial strain comprising a 16S rDNA sequence of SEQ ID NO: 9 may be referred to by the 16S rDNA sequence of SEQ ID NO: 44.

The bacterial strain comprising a 16S rDNA sequence of SEQ ID NO: 10 may be referred to by the 16S rDNA sequence of SEQ ID NO: 45.

The bacterial strain comprising a 16S rDNA sequence of SEQ ID NO: 11 disclosed herein has the highest homology with a bacterial strain of the species Parabacteroides distasonis, whereas based on whole genome sequence, the species with the highest homology is Parabacteroides sp. CAG:2. The bacterial strain comprising a 16S rDNA sequence of SEQ ID NO: 11 may be referred to as a bacterial strain of the species Parabacteroides distasonis, but, in addition or alternatively, may be referred to as Parabacteroides sp. CAG:2. The bacterial strain comprising a 16S rDNA sequence of SEQ ID NO: 11 may be referred to by the 16S rDNA sequence of SEQ ID NO: 46.

Thus, it should be appreciated that, in one aspect, the disclosure also provides compositions and methods comprising bacterial species with close homology to the bacterial strains that have a 16S rDNA sequence with a nucleic acid sequence selected from the group consisting of SEQ ID NOs: 1-11. Homologies based on 16S sequence analysis and whole genome analysis are presented in Table 1.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman. Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group. Madison. Wis.), or by manual alignment and visual inspection (see. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

It should be appreciated that the terms “bacteria” and “bacterial strains” as used herein are interchangeable. The compositions described herein containing multiple purified bacterial strains may also be referred to as “live bacterial products.”

In some embodiments of the compositions provided herein, one or more of the bacterial strains are human-derived bacteria, meaning the one or more bacterial strains were obtained from or identified from a human or a sample therefrom (e.g., a human donor). In some embodiments of the compositions provided herein, all of the bacterial strains are human-derived bacteria. In some embodiments of the compositions provided herein, the bacterial strains are derived from more than one human donor.

The bacterial strains used in the compositions provided herein generally are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated and grown up individually. The bacterial compositions that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals).

In some embodiments of the compositions provided herein, the composition includes one or more anaerobic bacteria. In some embodiments of the compositions provided herein, the composition includes only anaerobic bacteria. In some embodiments of the compositions provided herein, the composition includes one or more facultative anaerobic bacteria. In some embodiments of the compositions provided herein, the composition includes only facultative anaerobic bacteria. In some embodiments of the compositions provided herein, the composition includes one or more obligate anaerobic bacteria. In some embodiments of the compositions provided herein, the composition includes only obligate anaerobic bacteria.

In some embodiments of the compositions provided herein, one or more of the bacterial strains is a spore-former. In some embodiments of the compositions provided herein, one or more of the bacterial strains is in spore form. In some embodiments of the compositions provided herein, one or more of the bacterial strains is a non-spore former.

In some embodiments, the compositions described herein comprise spore forming and non-spore forming bacterial strains. In some embodiments, the compositions described herein comprise spore-forming bacterial strains. In some embodiments, the compositions described herein comprise only spore-forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore format may germinate when they are introduced in the intestine.

In some embodiments, at least one (e.g., 1, 2, 3, 4, 5, or more) of the bacterial strains in the composition is a spore former. In some embodiments, at least one (e.g., 1, 2, 3, 4, 5, or more) of the bacterial strains in the composition is in spore form. In some embodiments, at least one (e.g., 1, 2, 3, 4, 5, or more) of the bacterial strains in the composition is a non-spore former. In some embodiments, at least one (e.g., 1, 2, 3, 4, 5, or more) of the bacterial strains in the composition is in vegetative form (as discussed above, spore forming bacteria can also be in vegetative form). In some embodiments, at least one (e.g., 1, 2, 3, 4, 5, or more) of the bacterial strains in the composition is in spore form and at least one (e.g., 1, 2, 3, 4, 5, or more) of the bacterial strains in the composition is in vegetative form. In some embodiments, at least one bacterial strain that is considered able to form spores (i.e., a spore-former) but is present in the composition in vegetative form. In some embodiments, at least one bacterial strain that is considered able to form spores is present in the composition both in spore form and in vegetative form.

It is envisioned that the bacterial strains of the compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

In any of the compositions provided herein, in some embodiments, the bacterial strains are purified. In any of the compositions provided herein, in some embodiments, the bacterial strains are isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals. As used herein, the term “isolated” refers to bacteria that have been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected. As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

The methods described herein involve administering any of the pharmaceutical compositions described herein to a subject in need thereof. As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human. In some embodiments, the human subject is a neonatal subject, a pediatric subject, an adolescent subject, an adult subject, or a geriatric subject. In some embodiments, the subject has or is at risk of having cancer. In some embodiments, the subject has or is at risk of having colitis associated with immune checkpoint inhibitor therapy. In some embodiments, the subject is in need of colonization of a microbiome. In some embodiments, the subject is in need of restoration of a microbiome. In some embodiments, the subject has experienced a dysbiosis-inducing event (e.g., treatment with an antibiotic) and is in need of restoration of a microbiome.

Any of the compositions described herein may be administered to a subject in a therapeutically effective amount or a dose of a therapeutically effective amount to treat or prevent cancer. In some embodiments, any of the compositions described herein may be administered to a subject in a therapeutically effective amount or a dose of a therapeutically effective amount to treat or prevent colitis associated with immune checkpoint inhibitor therapy. The terms “treat” or “treatment” refer to reducing or alleviating one or more of the symptoms associated with a disease or disorder (e.g., cancer, colitis associated with immune checkpoint inhibitor therapy). The terms “prevent” or “prevention” encompass prophylactic administration and may reduce the incidence or likelihood of experiencing a disease or disorder (e.g., cancer, colitis associated with immune checkpoint inhibitor therapy).

As used herein, the term “therapeutically effective amount” may be used interchangeably with the term “effective amount.” A therapeutically effective amount or an effective amount of a composition, such as a pharmaceutical composition, as described herein, is any amount that results in a desired response or outcome in a subject, such as those described herein. In some embodiments, the therapeutically effective amount is an amount sufficient to treat cancer, enhance efficacy of an immune checkpoint inhibitor therapy, treat colitis associated with an immune checkpoint inhibitor therapy, restore the microbiome, colonize the microbiome of a subject, induce a level of regulatory T cells, or induce a level of IFNγ or IFNγ+ CD8+ T cells.

It should be appreciated that the term “effective amount,” in reference to a composition comprising bacterial strains, may be expressed as the number of bacteria or CFUs to be administered. It should further be appreciated that the bacteria can multiply once administered. Thus, administration of even a relatively small amount of bacteria may have therapeutic effects.

In some embodiments, the compositions provided herein are effective in enhancing a checkpoint inhibitor therapy. In some embodiments, the present disclosure relates to methods for treating colitis. In some embodiments, the method further comprises administering one or more additional administrations of the pharmaceutical compositions described herein. In some embodiments, the method further comprises administering an antibiotic (e.g., vancomycin, penicillin, streptomycin) to the subject prior to administration of the pharmaceutical compositions.

As used herein, the term “enhance immune checkpoint inhibitor therapy” refers to improving and/or accelerating efficacy of the immune checkpoint inhibitor therapy, reducing or minimizing adverse events (e.g., side effects) associated with the immune checkpoint inhibitor therapy. In some embodiments, the compositions described herein when administered in combination with immune checkpoint inhibitor therapy increase the efficacy of the immune checkpoint inhibitor. In some embodiments, the compositions described herein when administered in combination with immune checkpoint inhibitor therapy increases the efficacy of the immune checkpoint inhibitor while simultaneously minimizing adverse events associated with immune checkpoint inhibitor therapy. Thus, in some embodiments, the disclosure provides methods and compositions that minimize adverse events that are caused by administration of an anticancer therapy (e.g., an immune checkpoint inhibitor). The term “adverse event” may be used interchangeably with the term “side effect” or toxicity” and refers to any undesired effect that is caused by administration of the anticancer therapy. An adverse event is considered to be caused by the anticancer therapy if the adverse event occurs subsequent to the initiation of the anticancer therapy. In general, the adverse event may be directly or indirectly caused by the anticancer therapy. In some embodiments, the adverse event is used as an indicator that the anticancer therapy is effective.

Examples of adverse events that may be caused by anticancer therapy include, without limitation, undesired immune responses, colitis, inflammation, dermatological toxicity, diarrhea, nausea, fatigue, hepatotoxicity, hypophysitis, eosinophilia, and autoimmune thyroid disease. Additional adverse events caused by the anticancer therapy will be evident to one of skill in the art. In some embodiments, the adverse event associated with immune checkpoint inhibitor therapy is colitis.

In some embodiments, the therapeutically effective amount of any of the compositions described herein is an amount sufficient to treat the disease, e.g., enhance survival of the subject, suppress an infection and/or treat the cancer.

Any of the methods described herein may be for the treatment of cancer in a subject. As used herein, methods of treating cancer involve relieving or alleviating at least one symptom associated with the cancer, or slowing or reversing the cancer progression. A method of treating cancer may, for example, eliminate or reduce a subject's tumor burden, reduce the number or replication of cancer cells, and/or prevent, delay or inhibit metastasis.

In some embodiments of the methods provided herein, the subject has cancer. In some embodiments of the methods provided herein, the cancer is carcinoma, glioma, mesothelioma, melanoma, lymphoma, leukemia, adenocarcinoma, breast cancer, ovarian cancer, cervical cancer, glioblastoma, multiple myeloma, prostate cancer, Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumors, or rhabdomyosarcoma. In some embodiments of the methods provided herein, the cancer is prostate cancer, bladder cancer, non-small cell lung cancer, urothelial carcinoma, melanoma, or renal cell carcinoma. In some embodiments of the methods provided herein, the subject is undergoing radiation treatment.

In some embodiments, the subject has been administered one or more anticancer agents. In some embodiments of the methods provided herein, the anticancer agent is a chemotherapy agent. In some embodiments of the methods provided herein, the anticancer agent is a cancer immunotherapy agent.

In some embodiments, the cancer immunotherapy agent is an immune checkpoint inhibitor. In some embodiments, the cancer immunotherapy agent is an immune checkpoint inhibitor.

In some embodiments of the compositions provided herein, the immune checkpoint inhibitor is a PD-1 inhibitor, PD-L-1 inhibitor, or CTLA-4 inhibitor. In some embodiments of the compositions provided herein, the immune checkpoint inhibitor is a PD-1 inhibitor, PD-L-1 inhibitor, CTLA-4 inhibitor, IDO1 inhibitor, VISTA inhibitor, LAG3 inhibitor, TIM3 inhibitor, PD-L-2 inhibitor, ARG1 inhibitor, B7 family inhibitory ligand B7-H3 inhibitor, B7 family inhibitor ligand B7-H4 inhibitor, 2B4 inhibitor, BLTA inhibitor, A2aR inhibitor, STAT3 inhibitor, or KIR inhibitor.

In some embodiments of the compositions provided herein, the immune checkpoint inhibitor is a PD-1 inhibitor. In some embodiments, the PD-1 inhibitor is nivolumab (OPDIVO®, Bristol Myers Squibb and Ono Pharmaceutical). In some embodiments, the PD-1 inhibitor is pembrolizumab (KEYTRUDA®, Merck). In some embodiments, the PD-1 inhibitor is pidiluzimab (CT-011, Curetech). In some embodiments, the PD-1 inhibitor is cemiplimab. In some embodiments, the PD-1 inhibitor is spartalizumab. In some embodiments, the PD-1 inhibitor is camrelizumab. In some embodiments, the PD-1 inhibitor sintilimab. In some embodiments, the PD-1 inhibitor is tislelizumab. In some embodiments, the PD-1 inhibitor is toripalimab. In some embodiments, the PD-1 inhibitor is AMP-224. In some embodiments, the PD-1 inhibitor is AMP-514.

In some embodiments, the PD-1 inhibitor is nivolumab (OPDIVO®). Nivolumab is a human IgG4 monoclonal antibody which inhibits the binding of PD-1 and its ligands PD-L1 and PD-L2. A detailed description of the properties and mechanism of action of nivolumab can be found in, for example, in PCT Publication No. WO 2013/173223, the contents of which are incorporated herein in their entirety. Briefly, nivolumab is a checkpoint inhibitor which blocks PD-1 ligand (PD-L1) binding to PD-1. Nivolumab is thought to bind primarily to the surface-exposed N-loop in PD-1 with high affinity and inhibit the interaction with both PD-L1 and PD-L2, thereby stimulating regulatory T cells against antigen-specific T cell proliferation. Nivolumab has been approved in the United States for the treatment of melanoma, squamous lung cancer, Hodgkin's Lymphoma, head and neck cancer, urothelial carcinoma, colorectal cancer, hepatocellular carcinoma, and metastatic non-small cell lung cancer.

In some embodiments, a PD-1 inhibitor is pembrolizumab (KEYTRUDA®). A detailed description of the properties and mechanism of action of pembrolizumab can be found in U.S. Pat. Nos. 8,354,509 and 8,900,587, the contents of which are incorporated herein in their entirety. Briefly, pembrolizumab binds and inhibits the activity of PD-1 on T cells, allowing the immune system to recognize and destroy cancer cells. Pembrolizumab is a humanized IgG4 immunoglobulin monoclonal antibody which binds primarily to the surface-exposed CD loop in PD-1 with high affinity and inhibits interaction with its ligands PD-L1 and PD-L2. Pembrolizumab has been approved in the United States for the treatment of metastatic melanoma, non-small cell lung cancer, head and neck cancer, Hodkgin's Lymphoma, urothelial carcinoma, gastric cancer, and cervical cancer.

In some embodiments, the PD-1 inhibitor is a PD-L1 inhibitor.

In some embodiments, the immune checkpoint inhibitor is a PD-L-1 inhibitor. In some embodiments, the PD-L-1 inhibitor is atezolizumab (TECENTRIQ®, Genentech and Roche). In some embodiments, the PD-L-1 inhibitor is avelumab (BAVENCIO®, Merck KGaA, Pfizer, and Eli Lilly). In some embodiments, the PD-L-1 inhibitor is durvalumab (IMFINZI®, MedImmune and AstraZeneca). Additional examples of PD-L-1 inhibitors include KN035, CK-301, AUNP12, CA-170, BMS-986189, MPDL3280A (RG7446, Roche), MEDI4736 (AstraZeneca), or MSB0010718C (Merck Serono).

In some embodiments, the immune checkpoint inhibitor is a CTLA-4 inhibitor. In some embodiments, the CTLA-4 inhibitor is an anti-CTLA-4 antibody. Examples of anti-CTLA-4 antibodies include, without limitation, ipilimumab, tremelimumab (CP-675,206), 9H10, 4F10, and 9D9. In some embodiments, the CTLA-4 inhibitor is ipilimumab. In some embodiments, the CTLA-4 inhibitor is tremelimumab. It should further be appreciated that multiple anticancer agents (e.g., immune checkpoint inhibitors) may be included in the compositions and methods disclosed herein. For instance, in a non-limiting example, the compositions and methods disclosed include both a PD-1 inhibitor and a CTLA-4 inhibitor.

In some embodiments, the immune checkpoint inhibitor is an IDO1 inhibitor. In some embodiments, the IDO1 inhibitor is indoximod, epacadostat, or BMS-986205.

In some embodiments, the immune checkpoint inhibitor is a VISTA inhibitor. In some embodiments, the VISTA inhibitor is CA-170 or JNJ-61610588.

In some embodiments, the immune checkpoint inhibitor is a LAG3 inhibitor. In some embodiments, the LAG3 inhibitor is IMP321, retaltimab, or GSK2831781.

In some embodiments, the immune checkpoint inhibitor is a TIM3 inhibitor. In some embodiments, the TIM3 inhibitor is MBG453, TSR-022, or LY3321367.

In some embodiments of the compositions provided herein, the immune checkpoint inhibitor is a PD-L-2 inhibitor.

In some embodiments, the immune checkpoint inhibitor is an ARG-1 inhibitor. In some embodiments, the ARG-1 inhibitor is CB-1158, INCB00158, OAT-1746, or K567-Arginase I.

In some embodiments, the immune checkpoint inhibitor is a B7 family inhibitory ligand (B7-H3) inhibitor. In some embodiments, the B7-H3 inhibitor is enoblituzumab, MGA271, or DS-5573a.

In some embodiments, the immune checkpoint inhibitor is a B7 family inhibitor ligand (B7-H4) inhibitor. In some embodiments, the B7-H4 inhibitor is FPA150.

In some embodiments, the immune checkpoint inhibitor is a 2B4 inhibitor.

In some embodiments, the immune checkpoint inhibitor is a BTLA inhibitor.

In some embodiments, the immune checkpoint inhibitor is an A2aR inhibitor. In some embodiments the A2aR inhibitor is NIR178, ATL-444, istradefyline, MSX-3, preladenant, SCH-58261, SCH-412,348, SCH-442,416, VER-6623, VER-6947, VER-7835, vipadenant or ZM-241,385.

In some embodiments, the immune checkpoint inhibitor is a STAT3 inhibitor. In some embodiments the STAT3 inhibitor is Sant7.

In some embodiments, the immune checkpoint inhibitor is a KIR inhibitor. In some embodiments the KIR inhibitor is 1-7F9 or lirilumab.

Adverse events associated with checkpoint inhibitor therapy are summarized for instance in Postow et al., Up to Date June 2017, Toxicities associated with checkpoint inhibitor immunotherapy, authors Postow M and Wolchok J, editors Atkins M and Ross M., Yang et al., Recognizing and managing toxicities in cancer immunotherapy, Tumor Biology March (2017): 1-13; and Linardou et al., Toxicity management of immunotherapy for patients with metastatic melanoma. Ann Transl. Med (2016) 4 (14) 22, which are all incorporate herein by reference in their entirety. In some embodiments, the subject has been administered one or more immune checkpoint inhibitors and has experienced or is at risk of immune checkpoint inhibitor-induced colitis (See, e.g., Prieux-Clotz et al., Target Oncology (2017) 12, 301-308).

Any of the compositions described herein may further comprise one or more anticancer agents described herein. In some embodiments, the composition further comprises one or more immune checkpoint inhibitors. In some embodiments, the composition further comprises a PD-1 inhibitor, PD-L-1 inhibitor, or CTLA-4 inhibitor. In some embodiments the composition further comprises a PD-1 inhibitor, PD-L-1 inhibitor, CTLA-4 inhibitor, IDO1 inhibitor, LAG3 inhibitor, or TIM3 inhibitor. In some embodiments of the methods provided herein, the immune checkpoint inhibitor is a PD-1 inhibitor. In some embodiments of the methods provided herein, the immune checkpoint inhibitor is a CTLA-4 inhibitor. In some embodiments of the methods provided herein, the cancer immunotherapy agent is a PD-1 inhibitor and a CTLA-4 inhibitor.

In some embodiments, the compositions provided herein are effective in treating colitis associated with checkpoint inhibitor therapy. In some embodiments, the present disclosure relates to methods for treating colitis. In some embodiments, the method further comprises administering one or more additional administrations of the pharmaceutical compositions described herein. In some embodiments, the method further comprises administering an antibiotic (e.g., vancomycin, penicillin, streptomycin) to the subject prior to administration of the pharmaceutical compositions.

Colitis is a digestive disease characterized by inflammation of the inner lining of the colon. Typical symptoms of colitis include, for example, abdominal pain, cramping, bloating, blood in stool, canker sores, chills, fatigue, joint swelling, malaise, mouth ulcers, rectal ulcers, skin sores, weakness, weight loss and diarrhea. In general, colitis can be caused by a number of factors, such as bacterial infections, viruses, autoimmune disorders. In addition, colitis may be a side effect or adverse event associated with immune checkpoint therapies (ICT). (See e.g., Prieux-Clotz et al., Target Oncology (2017) 12, 301-308).

In addition to being an effective treatment for cancer, immune checkpoint inhibitor therapy may stimulate immune responses to attack normal, non-cancerous parts of the colon to cause colitis. Typically colitis onset occurs 5-10 weeks after the second or third dose of ICT treatment and is endoscopically indistinguishable from inflammatory bowel disease. Colitis caused by ICT is graded based on severity, wherein grade 1 is characterized by asymptomatic colitis and <4 stools per day over baseline; grade 2 is characterized by abdominal pain, mucus, blood in the stood, and 4-6 stools per day; grade 3 is characterized by severe pain, fever, peritoneal signs, and at least 7 stools per day; grade 4 is characterized by life-threatening consequences such as perforation, ischemia, necrosis, bleeding, toxic megacolon, and hemodynamic collapse; and grade 5 is characterized by death. Conventional treatment of colitis associated ICT depends on the severity of the colitis is, for example, for mild colitis, the subject may be monitored closely without changes in treatment. However, for moderate colitis, the subject may be prescribed an immunosuppressive medication (e.g., corticosteroids, infliximab, vedolizumab), and for severe colitis, the ICT may be held or discontinued.

In some embodiments, the compositions and methods described herein reduce one or more symptom associated with the colitis associated with ICT. In some embodiments, the compositions and methods described herein reduce the severity of the colitis associated with ICT. In some embodiments, the compositions and methods described herein shorten the duration of the colitis associated with ICT.

In some embodiments, the bacterial strains of the compositions provided herein can treat colitis associated with ICT because of the synergy between the bacterial strains. Thus, without being limiting, in some embodiments, the combination of the bacterial strains of the compositions provided herein act synergistically because the combination of the strains, for example, induces a sufficient level of immune responses in the subject to reduce or prevent colitis associated with ICT. In some embodiments, the combination of the strains, for example, induces a sufficient level of regulatory T cells in the subject to reduce or prevent colitis associated with ICT. In some embodiments, the combination of the strains, for example, induces a sufficient level of IFNγ and/or IFNγ+ CD8+ T cells in the subject to reduce or prevent colitis associated with ICT.

In some embodiments, the compositions provided herein induce the proliferation and/or accumulation of regulatory T cells in the subject. As will be evident to one of ordinary skill in the art, regulatory T cells, also referred to as “Tregs,” are a subset of T lymphocytes that are generally thought to suppress an abnormal or excessive immune response and play a role in immune tolerance. Regulatory T cells may be identified based expression of the markers Foxp3 and CD4 (Foxp3+CD4+). The term regulatory T cells may also include Foxp3-negative regulatory T cells that are IL-10-producing CD4-positive T cells.

In some embodiments, the therapeutically effective amount is an amount sufficient to induce the proliferation and/or accumulation of Tregs in the subject (or in a sample obtained from a subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of Tregs in a subject (e.g., a subject with a pathogenic infection) that has not received any of the compositions described herein or as compared to a fecal sample from the same subject that was collected prior to administration of any of the compositions.

As used herein, the phrase “induces proliferation and/or accumulation of regulatory T cells” refers to an effect of inducing the differentiation of immature T cells into regulatory T cells, which differentiation leads to the proliferation and/or the accumulation of regulatory T cells. Further, the meaning of “induces proliferation and/or accumulation of regulatory T cells” includes in vivo effects, in vitro effects, and ex vivo effects. In some embodiments, the proliferation and/or accumulation of regulatory T cells may be assessed by detecting and/or quantifying the number of cells that express markers of regulatory T cells (e.g., Foxp3 and CD4), for example by flow cytometry. In some embodiments, the proliferation and/or accumulation of regulatory T cells may be assessed by determining the activity of the regulatory T cells, such as the production of cytokines (e.g., IL-10).

In some embodiments, administration of the compositions described herein results in an increase in the proliferation and/or accumulation of regulatory T cells (e.g., total Tregs or antigen-specific Tregs) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or antigen-specific Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in another subject (e.g., a reference subject) who did not receive the compositions.

In some embodiments, administration of the compositions described herein results in an increase in the proliferation and/or accumulation of regulatory T cells (e.g., total Tregs or antigen-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or antigen-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in another subject (e.g., a reference subject) who did not receive the compositions.

In some embodiments, administration of the compositions described herein results in an increase in activity of regulatory T cells (e.g., total Tregs) at a particular site (e.g., the gastrointestinal tract) in the subject. In some embodiments, administration of the compositions described herein results in an increase in activity of regulatory T cells (e.g., total Tregs) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in the subject (or particular site in the subject) prior to administration of the compositions. In some embodiments, administration of the compositions described herein results in an increase in activity of regulatory T cells (e.g., total Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of regulatory T cells in another subject (e.g., a reference subject) who did not receive the compositions.

In some embodiments, administration of the compositions described herein results in an increase in the activity of regulatory T cells (e.g., total Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in the subject (or particular site in the subject) prior to administration of the compositions. In some embodiments, administration of the compositions described herein results in an increase in the activity of regulatory T cells (e.g., total Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in another subject (e.g., a reference subject) who did not receive the compositions.

The abundance of regulatory T cells (e.g., total Tregs) can be assessed by any method known in the art, for example by detecting a cellular marker indicative of regulatory T cells (e.g., FoxP3), assessing a direct or indirect activity of regulatory T cells, and/or by measuring the production of one or more cytokines produced by regulatory T cells (e.g., IL-10).

In some embodiments, the induction of regulatory T cells by the compositions provided herein results in the suppression or treatment of autoimmune diseases. In some embodiments, the induction of regulatory T cells by the compositions provided herein results in the suppression or treatment of colitis. In some embodiments, the induction of regulatory T cells by the compositions provided herein results in the suppression or treatment of colitis associated with anti-cancer treatment. In some embodiments, the induction of regulatory T cells by the compositions provided herein results in the suppression or treatment of colitis associated with treatment with immune checkpoint anti-cancer treatment.

In some embodiments, the compositions provided herein induce the production of IFNγ and/or the proliferation and/or accumulation of IFNγ+CD8+ T cells in the subject. As will be evident to one of ordinary skill in the art, IFNγ and IFNγ+CD8+ T cells are generally thought to play a role in the proinflammatory response to stimuli and aid in clearance of tumor cells. IFNγ may also be referred to and is also known in the art as IFNg, IFN-g, IFNgamma, or IFN-gamma.

In some embodiments, the therapeutically effective amount is an amount sufficient to induce the production of IFNγ and/or the proliferation and/or accumulation of IFNγ+ CD8+ T cells in the subject (or in a sample obtained from a subject) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the amount of IFNγ and/or IFNγ+CD8+ T cells in a subject that has not received any of the compositions described herein or as compared to a fecal sample from the same subject that was collected prior to administration of any of the compositions.

As used herein, the phrase “induces the proliferation and/or accumulation of IFNγ+CD8+ T cells” refers to an effect of inducing the differentiation of immature T cells into IFNγ+CD8+ T cells. Further, the meaning of “production of IFNγ and/or the proliferation and/or accumulation of IFNγ+CD8+ T cells” includes in vivo effects, in vitro effects, and ex vivo effects. In some embodiments, the proliferation and/or accumulation of IFNγ+CD8+ T cells may be assessed by detecting and/or quantifying the number of cells that express specific markers (e.g., IFNγ and CD8), for example by flow cytometry. In some embodiments, the proliferation and/or accumulation of IFNγ+CD8+ T cells may be assessed by determining the activity of the IFNγ+CD8+ T cells, such as the production of cytokines (e.g., IFNγ+). As used herein, the phrase “induces the production of IFNγ” refers to an effect of increasing the expression of the gene encoding IFNγ and/or increasing the amount of IFNγ protein produced.

In some embodiments, administration of the compositions described herein results in an increase in the production of IFNγ and/or the proliferation and/or accumulation of IFNγ+CD8+ T cells by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of IFNγ and/or IFNγ+CD8+ T cells in the subject (or particular site in the subject) prior to administration of the compositions. In some embodiments, administration of the compositions described herein results in an increase the production of IFNγ and/or the proliferation and/or accumulation of IFNγ+CD8+ T cells by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of IFNγ and/or IFNγ+CD8+ T cells in another subject (e.g., a reference subject) who did not receive the compositions.

In some embodiments, administration of the compositions described herein results in an increase in the production of IFNγ and/or the proliferation and/or accumulation of IFNγ+CD8+ T cells by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of IFNγ and/or IFNγ+CD8+ T cells in the subject (or particular site in the subject) prior to administration of the compositions. In some embodiments, administration of the compositions described herein results in an increase the production of IFNγ and/or the proliferation and/or accumulation of IFNγ+CD8+ T cells by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of IFNγ and/or IFNγ+CD8+ T cells in another subject (e.g., a reference subject) who did not receive the compositions.

In some embodiments, administration of the compositions described herein results in an increase in activity of IFNγ+CD8+ T cells at a particular site (e.g., the gastrointestinal tract) in the subject. In some embodiments, administration of the compositions described herein results in an increase in activity of IFNγ+CD8+ T cells by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of IFNγ+CD8+ T cells in the subject (or particular site in the subject) prior to administration of the compositions. In some embodiments, administration of the compositions described herein results in an increase in activity of IFNγ+CD8+ T cells by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the activity of IFNγ+CD8+ T cells in another subject (e.g., a reference subject) who did not receive the compositions.

In some embodiments, administration of the compositions described herein results in an increase in the activity of IFNγ+CD8+ T cells by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of IFNγ+CD8+ T cells in the subject (or particular site in the subject) prior to administration of the compositions. In some embodiments, administration of the compositions described herein results in an increase in the activity of IFNγ+CD8+ T cells by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of IFNγ+CD8+ T cells in another subject (e.g., a reference subject) who did not receive the compositions.

The abundance of IFNγ+CD8+ T cells can be assessed by any method known in the art, for example by detecting a cellular markers, assessing a direct or indirect activity of IFNγ+CD8+ T cells, and/or by measuring the production of one or more cytokines produced by IFNγ+CD8+ T cells (e.g., IFNγ).

In some embodiments, the compositions described herein induce an IFNγ+CD8+ T cell response that is sufficiently strong to promote clearance of tumor cells but not strong enough to induce colitis. In some embodiments, the compositions described herein induce an IFNγ+CD8+ T cell response that is sufficiently strong to promote clearance of tumor cells and a regulatory T cell response that is sufficiently strong to reduce or prevent colitis.

In some embodiments, the compositions provided herein restore the microbiome of a subject. In some embodiments, the present disclosure relates to methods for restoring the microbiome of a subject comprising administering a therapeutically effective amount of a pharmaceutical composition comprising one or more purified bacterial strains. In some embodiments, the method further comprises administering one or more additional administrations of the pharmaceutical compositions described herein. In some embodiments, the method further comprises administering an antibiotic (e.g., vancomycin, penicillin, streptomycin) to the subject prior to administration of the pharmaceutical compositions.

As used herein, the term “restoring the microbiome” of a subject refers to re-establishing or recovering the relative abundance of bacterial populations and/or microbial diversity of the gastrointestinal microbiome of the subject. In some embodiments, the microbiome of the subject is restored to a microbiome of a healthy subject (a healthy microbiome). In some embodiments, the microbiome of the subject is restored to the microbiome of the same subject at a previous time. In some embodiments, the subject whose microbiome is being restored may have an inflammatory condition, such as irritable bowel syndrome, ulcerative colitis, or Crohn's disease. In some embodiments, the microbiome of the subject is restored to the microbiome of the same subject prior to the inflammatory condition. Thus, it should be appreciated that a microbiome that is in need of restoring would not necessarily have undergone a dysbiosis inducing event. According to the methods provided herein, in some embodiments, the subject whose microbiome is in need of restoring may not have undergone a dysbiosis-inducing event. In some embodiments, the subject whose microbiome is in need of restoring has not been treated with an antibiotic. In some embodiments, the subject whose microbiome is being restored may have cancer. In some embodiments, the subject whose microbiome is being restored have received immune checkpoint inhibitor therapy.

A microbiome that is in need of restoration may be characterized, for example, by the presence of a signature associated with a damaged microbiome. In general, a microbiome may be characterized based on the presence or absence of populations of bacteria or specific bacterial species. For example, a damaged microbiome may be overpopulated with pathogens and/or have reduced presence of commensal bacteria. A damaged microbiome may also be characterized by its biological function. For example, a damaged microbiome may not be able to maintain the mucosal barrier and healthy immune function, and/or a damaged microbiome may not be able to prevent or combat infections as well as a healthy microbiome.

In some embodiments, restoring the microbiome of a subject results in a microbiome that is substantially similar to a healthy microbiome (e.g., a microbiome of a healthy subject). In some embodiments, restoring the microbiome of a subject results in a microbiome that is more similar to a healthy microbiome than to a damaged microbiome. In some embodiments, restoring the microbiome of a subject results in a restoration or increased presence of one or more populations of bacteria or specific bacterial species. In some embodiments, restoring the microbiome of a subject results in a restoration or decreased presence of one or more populations of bacteria or specific bacterial species. In some embodiments, restoring the microbiome of a subject results in an increase of one or more functions of the microbiome. In some embodiments, restoring the microbiome of a subject results in a decrease of one or more functions of the microbiome.

In some embodiments, the compositions described herein can restore the microbiome without restoring the full diversity of the microbiome. Without wishing to be limited to a particular theory, it is thought that the microbiome is restored by the make-up of the specific bacterial strains administered in the compositions, the methods of treatment, and dosing regimens provided herein. Thus, a microbiome treated with the compositions described herein may show a signature of a healthy microbiome (e.g., including the presence of Bacteroidetes and Firmicutes and the absence of Proteobacteria) but may not have the high diversity that may be typically associated with a healthy microbiome.

As used herein, a “healthy microbiome,” refers to a microbiome from a subject who does not have overt disease (e.g., a healthy subject). Although the microbial composition of healthy microbiomes can vary widely, several trends have emerged which characterized healthy microbiomes. For example, the gastrointestinal microbiome may perform a number of metabolic and/or other molecular functions, including the metabolism of carbohydrates, lipids, and other nutrients which are performed by healthy microbiomes, regardless of the specific species composition. In some instances, the metabolic and molecular functions carried out by a healthy microbiome cannot be performed by the host subject, resulting in a symbiotic host-microbial relationship. Additionally, healthy microbiomes tend to be resilient to external (e.g., dietary or pharmaceutical) and/or internal (e.g., age, disease-state, stress, inflammation) changes in the subject. The resilience of a healthy microbiome can also be characterized by the ability and the rate at which a healthy state is restored after occurrence of a perturbation. Alternatively, or in addition, a healthy microbiome may be characterized by a high (e.g., greater than 75%) relative abundance of bacterial species from the phylum Firmicutes and genus Bacteroides relative to species from the phylum Proteobacteria.

In some embodiments, the bacterial strains of the compositions provided herein can restore the microbiome of a subject because of the synergy between the bacterial strains. Thus, without being limiting, in some embodiments, the combination of the bacterial strains of the compositions provided herein act synergistically because the combination of the strains is, for example, particularly well-suited to use nutrients in the intestinal tract (e.g., the colon or the cecum), or through metabolic interactions, and/or because the combination is superior in grafting (e.g., by providing a favorable microenvironment).

In some embodiments, the compositions provided herein colonize the microbiome of a subject. In some embodiments, the present disclosure relates to methods for colonizing the microbiome of a subject comprising administering a therapeutically effective amount of a pharmaceutical composition comprising one or more purified bacterial strains. In some embodiments, the methods further comprise administering one or more additional doses or amounts of the compositions described herein. In some embodiments, the methods further comprise administering an antibiotic to the subject prior to administration of the compositions. In some embodiments, the methods further comprise administering an antibiotic (e.g., vancomycin, penicillin, streptomycin) to the subject prior to administration of the compositions.

In some embodiments, one or more of the bacterial strains of the compositions provided herein colonize or recolonize the gastrointestinal tract or parts thereof (e.g., the colon or the cecum) of the subject. Such colonization may also be referred to as grafting or engraftment. In some embodiments, one or more of the bacterial strains of the compositions recolonize the intestinal tract (e.g., the colon or the cecum) of the subject after the naturally present microbiome has been partially or completely removed, e.g., due to a dysbiosis inducing event, such as immune checkpoint inhibitor therapy. In some embodiments, one or more of the bacterial strains of the compositions recolonize the intestinal tract (e.g., the colon or the cecum) of the subject after the naturally present microbiome has been partially or completely removed by antibiotic (e.g., vancomycin, penicillin, streptomycin) treatment. In some embodiments, one or more of the bacterial strains of the compositions colonize a dysbiotic gastrointestinal tract (e.g., a gastrointestinal tract of a subject that has received immune checkpoint inhibitor therapy, or a gastrointestinal tract that has undergone antibiotic treatment). In some embodiments, all of the bacterial strains of the composition colonize the gastrointestinal tract. In some embodiments, all of the bacterial strains of the compositions colonize a dysbiotic gastrointestinal tract. In some embodiments, multiple doses of the bacterial compositions are administered to allow for all of the bacterial strains of the composition to colonize the gastrointestinal tract. In some embodiments, multiple doses of the bacterial compositions are administered to allow for all of the bacterial strains of the compositions to colonize a dysbiotic gastrointestinal tract.

In some embodiments, the one or more bacterial strains of the pharmaceutical compositions colonize the microbiome because they can “outgrow” other bacterial strains (e.g., pathogens). In some embodiments, the subject has been treated with an antibiotic resulting in a removal of most of the microbiome, providing a “clean slate” environment for both the one or more bacterial strains of compositions and any other bacterial strains (e.g., pathogens). Thus, without being limited to a specific mechanism, if a pathogen and one or more bacterial strains of the compositions provided herein are both present in the intestinal tract (e.g., the colon or the cecum), the one or more bacterial strains of compositions provided herein grow faster (e.g., have a shorter doubling time) than the pathogen, thereby preventing the pathogen from accumulating in the intestinal tract (e.g., the colon or the cecum) and allowing the one or more bacterial strains of the compositions to colonize. In some embodiments, the faster growth results because the one or more bacterial strains of the compositions provided herein are better at grafting in the intestinal tract (e.g., the colon or the cecum). In some embodiments, the faster growth results because the one or more bacterial strains of the compositions provided herein are better at metabolizing nutrients present in the intestinal tract (e.g., the colon or the cecum). Thus, without being limiting, in some embodiments, the combination of the bacterial strains of the compositions provided herein act synergistically because the combination of the strains is particularly well-suited to use nutrients in the intestinal tract (e.g., the colon or the cecum), or through metabolic interactions, and/or because the combination is superior in grafting (e.g., by providing a favorable microenvironment). In some embodiments, the one or more bacterial strains of the compositions described herein are able to colonize specific niches in the intestinal tract (e.g., the colon or the cecum). In some embodiments, the one or more bacterial strains of the compositions described herein are able to colonize specific niches in the intestinal tract (e.g., the colon or the cecum) that became available after antibiotic treatment.

The extent of colonization of one or more of the bacterial strains may be determined, for example by detecting the presence of one or more bacterial strains and/or by quantifying the abundance of the one or more bacterial strains. In some embodiments, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the bacterial strains of the compositions colonize the microbiome of the subject. In some embodiments, at least 25% of the bacterial strains of the compositions colonize the microbiome of the subject. In some embodiments, at least 50% of the bacterial strains of the compositions colonize the microbiome of the subject. In some embodiments, 100% of the bacterial strains of compositions colonize the microbiome of the subject. In some embodiments, the percentage of the bacterial strains of the compositions that colonize the microbiome of the subject is increased by administering additional doses of the compositions.

The extent of colonization of the microbiome by the one or more bacterial strains of the compositions described herein may be based on the relative abundance of the bacterial strains of the compositions in the microbiome. For example, in some embodiments, at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the bacterial strains of the microbiome of the subject are the bacterial strains of the compositions. In some embodiments, at least 25% of the bacterial strains detected in the microbiome of the subject are the bacterial strains of the compositions. In some embodiments, at least 50% of the bacterial strains detected in the microbiome of the subject are the bacterial strains of the compositions. In some embodiments, the percentage of the bacterial strains of the bacterial compositions in the microbiome of the subject is increased by administering additional doses of the compositions.

In some embodiments, the compositions described herein result in durable colonization by one or more bacterial strains in the composition. In some embodiments, one or more bacterial strains of the compositions described herein are detected in the microbiome of the subject for an extended period of time. In some embodiments, one or more bacterial strains of the compositions described herein colonize the microbiome of the subject for an extended period of time. In some embodiments, one or more bacterial strains of the compositions described herein are detected in the microbiome of the subject at least one week, two weeks, three weeks, four weeks, five weeks, or six weeks, seven weeks, eight weeks, nine weeks, ten weeks, eleven weeks, twelve weeks, three months, four months, five months, six months, seven months, eight months, nine months, ten months, eleven months, one year or longer following administration of the composition.

It should be appreciated that in one aspect, the methods provided herein provide better colonization (e.g., engraftment of a higher number/percentage and/or higher abundance) and more durable colonization of the bacterial strains of the pharmaceutical compositions when compared to the colonization of the same pharmaceutical compositions administered according to different methods. Thus, in one aspect, the colonization is better and more durable because the pharmaceutical compositions are administered according to the specific dose regimens, amounts, and/or antibiotic treatment regimens provided herein.

It should be appreciated that in one aspect, the methods provided herein provide more durable colonization of the bacterial strains of the pharmaceutical compositions when compared to the colonization of the same pharmaceutical compositions administered according to different methods. Thus, in one aspect, the colonization is more durable because the pharmaceutical compositions are administered according to the specific dose regimens, amounts, and/or antibiotic treatment regimens provided herein.

In some embodiments, the compositions described herein are administered to a subject concomitantly with one or more additional therapeutic agents. In some embodiments, the compositions described herein are administered to a subject followed by administration of one or more additional therapeutic agent. In some embodiments, any of the compositions described herein is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the one or more additional therapeutic agent. Alternatively, in some embodiments, one or more therapeutic agent administered to a subject followed by administration of any of the compositions described herein. In some embodiments, one or more therapeutic agent is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of any the compositions described herein.

Also within the scope of the present disclosure are compositions, e.g., compositions for administering to a subject, such as pharmaceutical compositions. In some embodiments, the composition comprises any of the bacterial strains described herein.

In one aspect, the disclosure provides pharmaceutical compositions comprising any of the bacterial strains described herein. In some embodiments, the pharmaceutical composition comprises a pharmaceutical acceptable excipient. In some embodiments, the pharmaceutical composition is formulated for oral administration. In some embodiments, the pharmaceutical composition is formulated for rectal administration. In some embodiments, the pharmaceutical composition is formulated for delivery to the intestine. In some embodiments, the pharmaceutical composition is formulated for delivery to the colon.

In some embodiments, the pharmaceutical compositions described herein contain one or more bacterial strains. In some embodiments, the pharmaceutical compositions may be lyophilized. In some embodiments, the pharmaceutical composition is in the form of a capsule. In some embodiments, the pharmaceutical composition further comprises a pH-sensitive composition comprising one or more enteric polymers.

Any of the compositions described herein, including the pharmaceutical compositions and food products comprising bacterial strains, the bacterial strains in any form, for example in an aqueous form, such as a solution or a suspension, embedded in a semi-solid form, in a powdered form, or freeze-dried form. In some embodiments, the composition or the bacterial strains are lyophilized. In some embodiments, a subset of the bacterial strains is lyophilized. Methods of lyophilizing compositions, specifically compositions comprising bacteria, are well known in the art. See, e.g., U.S. Pat. Nos. 3,261,761; 4,205,132; PCT Publication Nos WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may be subsequently be combined with a pharmaceutical excipient. In some embodiments, the bacterial strain is a lyophilized cake. In some embodiments, the compositions comprising the one or more bacterial strains are a lyophilized cake.

In some embodiments, one or more of the bacterial strains of the compositions, including pharmaceutical compositions and food products, has been spray-dried. In some embodiments, a subset of the bacterial strains is spray-dried. The process of spray-drying refers to production of dry powder from a liquid comprising bacterial compositions (See, e.g., Ledet, et al., Spray Draying of Pharmaceuticals in “Lyophilized Biologics and Vaccines” pages 273-294, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined, may be subsequently combined with a pharmaceutical excipient.

The bacterial strains can be manufactured using fermentation techniques well known in the art. In some embodiments, the bacteria are propagated or manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the live bacterial product may be formulated for administration as a pharmaceutical composition. The term “pharmaceutical composition” as used herein means a product that results from the mixing or combining of at least one active ingredient, such as any of the bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like. In some embodiments, the pharmaceutical compositions can be used by injection, such as by intravenous, intramuscular, subcutaneous, or intradermal administration.

In some embodiments, the compositions comprising bacterial strains are formulated for oral delivery. In some embodiments, the bacteria are formulated for delivery to the intestines (e.g., the small intestine and/or the colon). In some embodiments, the bacteria are formulated with an enteric coating that increases the survival of the bacteria through the harsh environment in the stomach. The enteric coating is one which resists the action of gastric juices in the stomach so that the bacteria which are incorporated therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS PharmSciTech (2016) 17 (1), 56-67).

The compositions comprising bacteria may also be formulated for rectal delivery to the intestine (e.g., the colon). Thus, in some embodiments, the bacterial compositions may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH-sensitive compositions, more specifically, buffered sachet formulations or enteric polymers that release their contents when the pH becomes alkaline after the enteric polymers pass through the stomach. When a pH-sensitive composition is used for formulating the pharmaceutical preparation, the pH-sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. (See e.g., Villena et al., Int J Pharm (2015) 487 (1-2): 314-9).

Also within the scope of the present disclosure are pharmaceutical compositions for administration by additional or alternative routes. In some embodiments, the pharmaceutical compositions are formulated for sublingual administration. In some embodiments, the pharmaceutical compositions are formulated for administration by injection.

In some embodiments, a pharmaceutical composition may include an additional component that enables efficient delivery of the compositions of the disclosure to a desired site, such as the gastrointestinal tract (e.g., the colon).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising bacterial strains are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration, e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions employed, the route of administration, the time of administration, the duration of the treatment, other drugs, compounds and/or materials used in combination with the particular compositions employed, the age, sex, weight, condition, general health, and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect (e.g., treatment of cancer, treatment of colitis associated with immune checkpoint inhibitor therapy, level of regulatory T cells, level of IFNγ and/or IFNγ+ CD8+ T cells, level of colonization of microbiome, level of microbiome restoration) is achieved. In general, effective doses of the compositions disclosed herein, for the prophylactic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, at least 10 times, at least 11 times, at least 12 times, at least 13 times, at least 14 times, or more. In some embodiments, any of the compositions described herein are administered the subject in multiple doses at a regular interval, such as every day, every 2 days, every 3 days, every 4 days, every 5 days, every 6 days, every week, every 2 weeks, every 4 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose of day 2, third dose on day 3, etc.).

In some embodiments, the subject is administered a single dose of an antibiotic prior to the administration of any of the bacterial compositions described herein. In some embodiments, the subject is administered multiple doses of an antibiotic prior to the administration of any of the bacterial compositions described herein. In some embodiments, the subject is administered at least 2, 3, 4, 5 or more doses of an antibiotic prior to the administration of any of the bacterial compositions described herein. In some embodiments, the subject is administered a dose of an antibiotic at substantially the same time as the administration of any of the bacterial compositions described herein. Examples of antibiotics that can be administered include, without limitation, kanamycin, gentamicin, colistin, metronidazole, vancomycin, clindamycin, fidaxomicin, penicillin, streptomycin, and cefoperazone.

In some embodiments, the subject is administered one or more doses of a cancer therapy prior to administration of any of the compositions described herein. In some embodiments, the subject is administered a single dose of an immune checkpoint inhibitor prior to administration of any of the compositions described herein. In some embodiments, the subject is administered multiple doses of an immune checkpoint inhibitor prior to the administration of any of the bacterial compositions described herein. In some embodiments, the subject is administered at least 2, 3, 4, 5 or more doses of an immune checkpoint inhibitor prior to the administration of any of the bacterial compositions described herein. In some embodiments, the subject is administered a dose of an immune checkpoint inhibitor at substantially the same time as the administration of any of the bacterial compositions described herein.

In some embodiments, the disclosure provides compositions comprising an antibiotic and an anticancer agent (e.g., an immune checkpoint inhibitor). In some embodiments, the disclosure provides compositions comprising vancomycin and an anticancer agent (e.g., an immune checkpoint inhibitor). In some embodiments, the disclosure provides compositions comprising penicillin and/or streptomycin and an anticancer agent (e.g., an immune checkpoint inhibitor). In some embodiments, the disclosure provides compositions comprising an antibiotic and any of the bacterial compositions described herein. In some embodiments, the disclosure provides compositions comprising vancomycin and any of the bacterial compositions described herein. In some embodiments, the disclosure provides compositions comprising an antibiotic, an anticancer agent (e.g., an immune checkpoint inhibitor), and any of the bacterial compositions described herein. In some embodiments, the disclosure provides compositions comprising vancomycin, an anticancer agent (e.g., an immune checkpoint inhibitor), and any of the bacterial compositions described herein. In some embodiments, the disclosure provides compositions comprising penicillin and/or streptomycin, an anticancer agent (e.g., an immune checkpoint inhibitor), and any of the bacterial compositions described herein.

In some embodiments, in any one of the methods provided herein an antibiotic is administered together with an anticancer agent (e.g., an immune checkpoint inhibitor). In some embodiments, in any one of the methods provided herein vancomycin is administered together with an anticancer agent (e.g., an immune checkpoint inhibitor). In some embodiments, in any one of the methods provided herein an antibiotic is administered together with any of the bacterial compositions described herein. In some embodiments, in any one of the methods provided herein vancomycin is administered together with any of the bacterial compositions described herein. In some embodiments, in any one of the methods provided herein an antibiotic is administered together with an anticancer agent (e.g., an immune checkpoint inhibitor) and any of the bacterial compositions described herein. In some embodiments, in any one of the methods provided herein vancomycin is administered together with an anticancer agent (e.g., an immune checkpoint inhibitor) and any of the bacterial compositions described herein. In some embodiments, in any one of the methods provided herein penicillin and/or streptomycin is administered together with an anticancer agent (e.g., an immune checkpoint inhibitor) and any of the bacterial compositions described herein.

In some embodiments in any one the methods provided herein, a subject is evaluated for the presence of one or more of the bacterial strains of the compositions described herein in the microbiome. In some embodiments, if the subject does not have, or only has a low level one or more of the bacterial strains of the bacterial compositions described herein in the microbiome, any one of the compositions provided herein, or one or more further doses of any one of the compositions provided herein, may be administered.

In some embodiments one of any of the methods provided herein, a subject is evaluated for the presence of one or more immune response. For example, in some embodiments, a subject is evaluated for the presence of CD8+ T cells. In some embodiments, a subject is evaluated for the presence of production of IFNγ. in some embodiments, a subject is evaluated for the presence of regulatory T cells. In some embodiments, if the subject does not have, or only has a low level of the immune response, any one of the compositions provided herein may be administered.

In some embodiments, the subject is evaluated for the presence of and/or abundance of one or more bacterial strains of the bacterial compositions described herein in the microbiome. In some embodiments, if one or more bacterial strains of the compositions are detected at a level above a threshold level, no further compositions or food products are administered to the subject. In some embodiments, if one or more bacterial strains of the compositions colonize the subject to a level above a threshold level, no further compositions or food products are administered to the subject.

In some embodiments, the subject is evaluated for the presence of and/or magnitude of one or more immune responses associated with administration of the bacterial compositions described herein. In some embodiments, if a level of CD8+ T cell response is detected above a threshold level, no further compositions or food products are administered to the subject.

In general, a “threshold level” refers to a predetermined level, a level above which is indicative of sufficient colonization and/or presence of the bacterial strains of the bacterial compositions described herein. In some embodiments, the threshold level refers to a level measured in a subject who does not have a specific disease or disorder (e.g., cancer, colitis associated with immune checkpoint inhibitor therapy). In some embodiments, the threshold level refers to a level measured in the same subject prior to administration of the bacterial compositions described herein.

The compositions, including the pharmaceutical compositions disclosed herein, include compositions that contain selected bacterial strains The amount of bacteria in the compositions, including the amount of bacteria of each of the bacterial strains, in the compositions, including pharmaceutical compositions, may be expressed in weight, number of bacteria and/or CFUs (colony forming units). In some embodiments, the pharmaceutical compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacteria of the composition per dosage amount. In some embodiments, the pharmaceutical compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that the bacteria of the compositions may be present in different amounts. Thus, for instance, as a non-limiting example, a composition may include 10³ of bacteria A, 10⁴ of bacteria B and 10⁶ of bacteria C. In some embodiments, the pharmaceutical compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacteria in the composition per dosage amount. In some embodiments, the pharmaceutical compositions disclosed herein contain about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacteria combined per dosage amount. As discussed above, bacteria of the compositions may be present in different amounts. In some embodiments, the pharmaceutical compositions disclosed herein contain about 10⁻⁷, about 10⁻⁶, about 10⁻⁵, about 10⁴, about 10⁻³, about 10⁻², about 10⁻¹ or more grams of each of the bacteria in the composition per dosage amount. In some embodiments, the pharmaceutical compositions disclosed herein contain about 10⁻⁷, about 10⁻⁶, about 10⁻⁵, about 10⁴, about 10⁻³, about 10⁻², about 10⁻¹ or more grams in total for all of the bacteria combined per dosage amount.

In some embodiment, the dosage amount is one administration device (e.g., one table, pill or capsule). In some embodiments, the dosage amount is the amount administered at one time, which may be in the form of more than one administration device (e.g., more than one table, pill or capsule). In some embodiment, the dosage amount is the amount that is administered in a particular period (e.g., one day or one week).

As described herein, any of the pharmaceutical compositions described herein may be administered once, as a single dose. In some embodiments, the pharmaceutical compositions described herein are administered in multiple doses. In some embodiments, each dose is administered in the form of one or more capsules. In some embodiments, each dose comprises administration of multiple capsules. In some embodiments, each dose is administered in the form of 1, 2, 3, 4, 5, 6, 7, 8, 9, 10, 11, 12, 13, 14, 15, or more capsules.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacterial strains per capsule.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per capsule. In some embodiments, each capsule contains between 10⁷ and 10⁹, between 10⁷ and 10⁸, or between 10⁸ and 10⁹ total bacteria. In some embodiments, each capsule contains about 1.0×10⁷, 2.0×10⁷, 3.0×10⁷, 4.0×10⁷, 5.0×10⁷, 6.0×10⁷, 7.0×10⁷, 8.0×10⁷, 9.0×10⁷, 1.0×10⁸, 2.0×10⁸, 3.0×10⁸, 4.0×10⁸, 5.0×10⁸, 6.0×10⁸, 7.0×10⁸, 8.0×10⁸, 9.0×10⁸, 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹ total bacteria. In some embodiments, each capsule contains about 8.0×10⁸ total bacteria. In some embodiments, each capsule contains about 1.6×10⁹ total bacteria. In some embodiments, each capsule contains about 8.0×10⁸ CFUs. In some embodiments, each capsule contains about 1.6×10⁹ CFUs.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each bacterial strain per capsule.

In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² CFUs of each of the bacterial strains per dosage amount. In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total CFUs per dosage amount.

As described herein, any of the pharmaceutical compositions described herein may be administered to a subject in one dose or in multiple doses (e.g., initial administration), which may be followed by one or more additional doses of any of the pharmaceutical compositions described herein. In some embodiments, any of pharmaceutical composition described herein may be administered to a subject in one dose or in multiple doses in an initial administration, followed by one or more additional doses of a pharmaceutical composition comprising the same one or more bacterial strains as the pharmaceutical composition of the initial administration. In some embodiments, any of pharmaceutical composition described herein may be administered to a subject in one dose or in multiple doses in an initial administration, followed by one or more additional doses of a pharmaceutical composition comprising more total bacteria (colony-forming units) relative to the initial administration of the pharmaceutical composition. In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition. In some embodiments, the initial administration includes at least 1, 2, 3, 4, 5, 6, 7, 8, 9, 10, 11, 12, 13, 14, 15 or more doses of any of the pharmaceutical compositions described herein. In some embodiments, the additional administration includes at least 1, 2, 3, 4, 5, 6, 7, 8, 9, 10, 11, 12, 13, 14, 15 or more doses of any of the pharmaceutical compositions described herein. In some embodiments, the initial administration comprises two doses of any of the pharmaceutical composition and the additional administration comprises three doses of any of the pharmaceutical compositions described herein.

In some embodiments, the subject has not received a dose of an antibiotic prior to administration of the bacterial composition. In some embodiments, the subject has not been administered an antibiotic at least 1, at least 2, at least 3, at least 5, at least 10, at least 15, at least 20, at least 25, at least 30, at least 60, at least 90, at least 120, at least 180 or at least 360 days prior to administration of the compositions provided herein. In some embodiments, the subject is treated with an amount of antibiotics sufficient to allow for the grafting of the one or more strains of the bacterial compositions provided herein.

In some embodiments, the subject may be administered one or more doses of an antibiotic prior to or concurrently with a bacterial composition. Antibiotics may be administered for a variety of reasons. For instance, antibiotics may be administered to remove bacterial species from the colon and/or intestine prior to administration of the bacterial compositions provided herein. Antibiotics may also be administered to suppress unwanted infections in the case of cancer treatment.

In some embodiments, the subject is administered a single dose of an antibiotic prior to the bacterial composition. In some embodiments, the subject is administered multiple doses of an antibiotic prior to the bacterial composition. In some embodiments, the subject is administered at least 2, 3, 4, 5 or more doses of an antibiotic prior to the bacterial composition. In some embodiments, the subject is administered a dose of an antibiotic at substantially the same time as the bacterial composition. Examples of antibiotics that can be administered include, without limitation, kanamycin, gentamicin, colistin, metronidazole, vancomycin, clindamycin, fidaxomicin, penicillin, streptomycin, and cefoperazone.

In one aspect, the disclosure provides methods comprising the administration of an antibiotic (e.g., vancomycin, penicillin, streptomycin) followed by the administration of a pharmaceutical composition provided herein, wherein the administration of an antibiotic (e.g., vancomycin, penicillin, streptomycin) is followed by the administration of a single dose or multiple doses of the pharmaceutical composition. In some embodiments, administration of an antibiotic (e.g., vancomycin, penicillin, streptomycin) followed by the administration of a single dose or multiple doses of the pharmaceutical composition results in an increase in the abundance of bacterial strains of the pharmaceutical compositions in the microbiome of the subject (engraftment) compared to the administration of a pharmaceutical composition without the administration of the antibiotic. In some embodiments, administration of an antibiotic (e.g., vancomycin, penicillin, streptomycin) followed by the administration of a single dose or multiple doses of the pharmaceutical composition results in an increase in the duration of the colonization of bacterial strains of the pharmaceutical composition in the microbiome of the subject (e.g., up to 6 months) compared to the administration of a pharmaceutical composition without the administration of the antibiotic.

In some embodiments, administration of an antibiotic (e.g., vancomycin) followed by the administration of multiple doses of the pharmaceutical composition results in an increase in the rate of engraftment of the initial amount of the bacterial strains of the pharmaceutical composition, a higher abundance of the bacterial strains of the pharmaceutical composition in the microbiome of the subject, an increase in the rate of engraftment of the initial amount of the bacterial strains of the pharmaceutical composition in the microbiome of the subject, results in a greater number (amount) of subjects having all of the bacterial strains of the pharmaceutical composition present in their microbiome, an accelerated recovery/restoration of the microbiome, and/or an accelerated colonization of the microbiome as compared to the administration of a single dose of the pharmaceutical composition.

In some embodiments, the methods described herein may involve subjecting the subject to a bowel lavage (bowel irrigation, whole bowel irrigation, gastrointestinal lavage, gastric lavage) prior to administration of the compositions described herein. In some embodiments, a bowel lavage may remove or aid in removing microbiota from the gastrointestinal tract of the subject, creating a niche for the bacterial strains of the compositions described herein. In some embodiments, the bowel lavage may be an oral bowel lavage or a rectal bowel lavage.

Methods of performing a bowel lavage are known in the art, and generally involve the rapid administration of large volumes of a solution, such as polyethylene glycol or a balanced electrolyte solution. A rectal bowel lavage can involve the administration of a solution or a suppository containing the pharmaceutical composition. A bowel lavage may be performed under doctor supervision, hospitalization, or at home.

Aspects of the present disclosure provide food products comprising any of the compositions provided herein and a nutrient. Also within the scope of the present disclosure are food products comprising any of the bacterial strains described herein and a nutrient. Food products are, in general, intended for the consumption of a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing bacterial strains described herein may be produced using methods known in the art and may contain the same amount of bacteria (e.g., by weight, amount or CFU) as the pharmaceutical compositions provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

TABLE 1
Bacterial Strains

| | SEQ | | Species with highest | NCBI | Species with highest |
|---|---|---|---|---|---|
| Strain | ID | Strain | homology based on | accession | homology based on |
| # | NO* | ID | original 16S analysis | ID | 16S resequencing |
| 2 | 2, 37 | 1A6 | Fusobacterium | KR822463 | Fusobacterium varium |
| | | | ulcerans | | |
| 7 | 7, 42 | 2F11 | Parabacteroides | NR041464 | Parabacteroides |
| | | | johnsonii | | johnsonii |
| 6 | 6, 41 | 2A6 | Paraprevotella | NR113078 | Paraprevotella |
| | | | xylaniphila | | xylaniphila |
| 11 | 11, 46 | 2G9 | Parabacteroides | NR041342 | Parabacteroides |
| | | | distasonis | | distasonis |
| 8 | 8, 43 | 1E7 | Alistipes sp. | LT223566 | Alistipes senegalensis |
| 10 | 10, 45 | 1C1 | Eubacterium limosum | NR113248 | Eubacterium limosum |
| 3 | 3, 38 | 1B11 | Bacteroides dorei | CP011531 | Bacteroides dorei |
| 9 | 9, 44 | 1H9 | Parabacteroides | NR112835 | Parabacteroides |
| | | | gordonii | | gordonii |
| 5 | 5, 40 | 2B1 | Subdolinogranulum | KM098109 | Gemminger formicilis |
| | | | sp. | | |
| 4 | 4, 39 | 2G1 | Bacteroides uniformis | NR112945 | Bacteroides uniformis |
| 1 | 1, 36 | 2G5 | Phascolarctobacterium | LN998073 | Phascolarctobacterium |
| | | | faecium | | faecium |

| | 16S | Species with highest | | | Alternative |
|---|---|---|---|---|---|
| | Identity | homology based on | WGS | WGS | species with |
| Strain | (%) of | whole genome | Identity | Coverage | high(est) |
| # | resequencing | sequencing (WGS) | (%) | (%) | homology |
| 2 | 99 | Fusobacterium | 93.2 | 78.6 | |
| | | ulcerans | | | |
| 7 | 99 | Parabacteroides | 99.9 | 90.5 | |
| | | johnsonii | | | |
| 6 | 99 | Paraprevotella | 98.9 | 92.1 | |
| | | xylaniphila | | | |
| 11 | 99 | Parabacteroides sp. | 99.4 | 95.4 | |
| | | CAG: 2 | | | |
| 8 | 99 | Alistipes senegalensis | 98.7 | 72.2 | Alistipes |
| | | | | | timonensis |
| 10 | 99 | Eubacterium limosum | 95 | 81 | |
| 3 | 99 | Bacteroides dorei | 99.3 | 79.5 | Bacteroides |
| | | | | | fluxus |
| 9 | 97 | Parabacteroides sp. | 90 | 50 | |
| | | HGS0025 | | | |
| 5 | 99 | Ruminococcaceae | 99.2 | 73.9 | Ruthenibacterium |
| | | bacterium cv2 | | | lactatiformans |
| 4 | 99 | Bacteroides sp. D20 | 98.5 | 81 | |
| 1 | 99 | Phascolarctobacterium | 99.2 | 87 | |
| | | sp. CAG: 207 | | | |

*For each of the bacterial strains, the strain may be referred to by either of the nucleic acid sequences provided by the referenced sequence identifier.

SEQUENCES

SEQ ID NO: 12G5_Phascolarctobacterium faecium_LN998073
GACGAACGCTGGCGGCGTGCCTAACACATGCAAGTCGAACGGAGAATTTTATTTCG
GTAGAATTCTTAGTGGCGAACGGGTGAGTAACGCGTAGGCAACCTACCCTTTAGAC
GGGGACAACATTCCGAAAGGAGTGCTAATACCGGATGTGATCATCTTGCCGCATGG
CAGGATGAAGAAAGATGGCCTCTACAAGTAAGCTATCGCTAAAGGATGGGCCTGCG
TCTGATTAGCTAGTTGGTAGTGTAACGGACTACCAAGGCGATGATCAGTAGCCGGT
CTGAGAGGATGAACGGCCACATTGGGACTGAGACACGGCCCAAACTCCTACGGGA
GGCAGCAGTGGGGAATCTTCCGCAATGGACGAAAGTCTGACGGAGCAACGCCGCG
TGAGTGATGAAGGATTTCGGTCTGTAAAGCTCTGTTGTTTATGACGAACGTGCAGTG
TGTGAACAATGCATTGCAATGACGGTAGTAAACGAGGAAGCCACGGCTAACTACGT
GCCAGCAGCCGCGGTAATACGTAGGTGGCGAGCGTTGTCCGGAATTATTGGGCGT
AAAGAGCATGTAGGCGGCTTAATAAGTCGAGCGTGAAAATGCGGGGCTCAACCCCG
TATGGCGCTGGAAACTGTTAGGCTTGAGTGCAGGAGAGGAAAGGGGAATTCCCAGT
GTAGCGGTGAAATGCGTAGATATTGGGAGGAACACCAGTGGCGAAGGCGCCTTTCT
GGACTGTGTCTGACGCTGAGATGCGAAAGCCAGGGTAGCGAACGGGATTAGATAC
CCCGGTAGTCCTGGCCGTAAACGATGGGTACTAGGTGTAGGAGGTATCGACCCCTT
CTGTGCCGGAGTTAACGCAATAAGTACCCCGCCTGGGGAGTACGGCCGCAAGGTT
GAAACTCAAAGGAATTGACGGGGGCCCGCACAAGCGGTGGAGTATGTGGTTTAATT
CGACGCAACGCGAAGAACCTTACCAAGGCTTGACATTGATTGAACGCTCTAGAGAT
AGAGATTTCCCTTCGGGGACAAGAAAACAGGTGGTGCATGGCTGTCGTCAGCTCGT
GTCGTGAGATGTTGGGTTAAGTCCCGCAACGAGCGCAACCCCTATCCTATGTTACC
AGCAAGTAAAGTTGGGGACTCATGGGAGACTGCCAGGGACAACCTGGAGGAAGGC
GGGGATGACGTCAAGTCATCATGCCCCTTATGTCTTGGGCTACACACGTACTACAA
TGGTCGGAAACAGAGGGAAGCGAAGCCGCGAGGCAGAGCAAACCCCAGAAACCCG
ATCTCAGTTCGGATCGCAGGCTGCAACCCGCCTGCGTGAAGTCGGAATCGCTAGTA
ATCGCAGGTCAGCATACTGCGGTGAATACGTTCCCGGGCCTTGTACACACCGCCCG
TCACACCACGAAAGTTGGTAACACCCGAAGCCGGTGAGGTAACCTA
SEQ ID NO: 21A6_Fusobacterium ulcerans_KR822463
GATGAACGCTGACAGAATGCTTAACACATGCAAGTCTACTTGATCCTTCGGGTGAA
GGTGGCGGACGGGTGAGTAACGCGTAAAGAACTTGCCTTACAGACTGGGACAACAT
TTGGAAACGAATGCTAATACCGGATATTATGATTGGGTCGCATGATCTGATTATGAA
AGCTATATGCGCTGTGAGAGAGCTTTGCGTCCCATTAGTTAGTTGGTGAGGTAACG
GCTCACCAAGACGATGATGGGTAGCCGGCCTGAGAGGGTGAACGGCCACAAGGGG
ACTGAGACACGGCCCTTACTCCTACGGGAGGCAGCAGTGGGGAATATTGGACAATG
GACCAAAAGTCTGATCCAGCAATTCTGTGTGCACGAAGAAGTTTTTCGGAATGTAAA
GTGCTTTCAGTTGGGAAGAAGTCAGTGACGGTACCAACAGAAGAAGCGACGGCTAA
ATACGTGCCAGCAGCCGCGGTAATACGTATGTCGCAAGCGTTATCCGGATTTATTG
GGCGTAAAGCGCGTCTAGGCGGCTTAGTAAGTCTGATGTGAAAATGCGGGGCTCAA
CCCCGTATTGCGTTGGAAACTGCTAAACTAGAGTACTGGAGAGGTAGGCGGAACTA
CAAGTGTAGAGGTGAAATTCGTAGATATTTGTAGGAATGCCGATGGGGAAGCCAGC
CTACTGGACAGATACTGACGCTAAAGCGCGAAAGCGTGGGTAGCAAACAGGATTAG
ATACCCTGGTAGTCCACGCCGTAAACGATGATTACTAGGTGTTGGGGGTCGAACCT
CAGCGCCCAAGCTAACGCGATAAGTAATCCGCCTGGGGAGTACGTACGCAAGTATG
AAACTCAAAGGAATTGACGGGGACCCGCACAAGCGGTGGAGCATGTGGTTTAATTC
GACGCAACGCGAGGAACCTTACCAGCGTTTGACATCCCAAGAAGTTAACAGAGATG
TTTTCGTGCCTCTTCGGAGGAACTTGGTGACAGGTGGTGCATGGCTGTCGTCAGCT
CGTGTCGTGAGATGTTGGGTTAAGTCCCGCAACGAGCGCAACCCCTTTCGTATGTT
ACCATCATTAAGTTGGGGACTCATGCGAGACTGCCTGCGATGAGCAGGAGGAAGGT
GGGGATGACGTCAAGTCATCATGCCCCTTATACGCTGGGCTACACACGTGCTACAA
TGGGTAGTACAGAGAGCTGCAAACCTGCGAGGGTAAGCTAATCTCATAAAACTATT
CTTAGTTCGGATTGTACTCTGCAACTCGAGTACATGAAGTTGGAATCGCTAGTAATC
GCAAATCAGCTATGTTGCGGTGAATACGTTCTCGGGTCTTGTACACACCGCCCGTC
ACACCACGAGAGTTGGTTGCACCTGAAGTAACAGGCCTAACCGTAA
SEQ ID NO: 3 1B11_Bacteroides dorei_CP011531
AGTTTGNNNTATGGCTCAGGATGAACGCTAGCTACAGGCTTAACACATGCAAGTCG
AGGGGCAGCATGGTCTTAGCTTGCTAAGGCTGATGGCGACCGGCGCACGGGTGAG
TAACACGTATCCAACCTGCCGTCTACTCTTGGCCAGCCTTCTGAAAGGAAGATTAAT
CCAGGATGGGATCATGAGTTCACATGTCCGCATGATTAAAGGTATTTTCCGGTAGAC
GATGGGGATGCGTTCCATTAGATAGTAGGCGGGGTAACGGCCCACCTAGTCAACGA
TGGATAGGGGTTCTGAGAGGAAGGTCCCCCACATTGGAACTGAGACACGGTCCAAA
CTCCTACGGGAGGCAGCAGTGAGGAATATTGGTCAATGGGCGATGGCCTGAACCA
GCCAAGTAGCGTGAAGGATGACTGCCCTATGGGTTGTAAACTTCTTTTATAAAGGAA
TAAAGTCGGGTATGCATACCCGTTTGCATGTACTTTATGAATAAGGATCGGCTAACT
CCGTGCCAGCAGCCGCGGTAATACGGAGGATCCGAGCGTTATCCGGATTTATTGG
GTTTAAAGGGAGCGTAGATGGATGTTTAAGTCAGTTGTGAAAGTTTGCGGCTCAAC
CGTAAAATTGCAGTTGATACTGGATGTCTTGAGTGCAGTTGAGGCAGGCGGAATTC
GTGGTGTAGCGGTGAAATGCTTAGATATCACGAAGAACTCCGATTGCGAAGGCAGC
CTGCTAAGCTGCAACTGACATTGAGGCTCGAAAGTGTGGGTATCAAACAGGATTAG
ATACCCTGGTAGTCCACACGGTAAACGATGAATACTCGCTGTTTGCGATATACGGC
AAGCGGCCAAGCGAAAGCGTTAAGTATTCCACCTGGGGAGTACGCCGGCAACGGT
GAAACTCAAAGGAATTGACGGGGGCCCGCACAAGCGGAGGAACATGTGGTTTAATT
CGATGATACGCGAGGAACCTTACCCGGGCTTAAATTGCACTCGAATGATCCGGAAA
CGGTTCAGCTAGCAATAGCGAGTGTGAAGGTGCTGCATGGTTGTCGTCAGCTCGTG
CCGTGAGGTGTCGGCTTAAGTGCCATAACGAGCGCAACCCTTGTTGTCAGTTACTA
ACAGGTGATGCTGAGGACTCTGACAAGACTGCCATCGTAAGATGTGAGGAAGGTGG
GGATGACGTCAAATCAGCACGGCCCTTACGTCCGGGGCTACACACGTGTTACAATG
GGGGGTACAGAGGGCCGCTACCACGCGAGTGGATGCCAATCCCTAAAACCCCTCT
CAGTTCGGACTGGAGTCTGCAACCCGACTCCACGAAGCTGGATTCGCTAGTAATCG
CGCATCAGCCACGGCGCGGTGAATACGTTCCCGGGCCTTGTACACACCGCCCGTC
AAGCCATGGGAGCCGGGGGTACCTGAAGTGCGTAACCGCGAGGAT
SEQ ID NO: 4 2G1_Bacteroides uniformis_NR_112945
GATGAACGCTAGCTACAGGCTTAACACATGCAAGTCGAGGGGCAGCATGAACTTAG
CTTGCTAAGTTTGATGGCGACCGGCGCACGGGTGAGTAACACGTATCCAACCTGCC
GATGACTCGGGGATAGCCTTTCGAAAGAAAGATTAATACCCGATGGCATAGTTCTTC
CGCATGGTAGAACTATTAAAGAATTTCGGTCATCGATGGGGATGCGTTCCATTAGGT
TGTTGGCGGGGTAACGGCCCACCAAGCCTTCGATGGATAGGGGTTCTGAGAGGAA
GGTCCCCCACATTGGAACTGAGACACGGTCCAAACTCCTACGGGAGGCAGCAGTG
AGGAATATTGGTCAATGGACGAGAGTCTGAACCAGCCAAGTAGCGTGAAGGATGAC
TGCCCTATGGGTTGTAAACTTCTTTTATACGGGAATAAAGTGAGGCACGTGTGCCTT
TTTGTATGTACCGTATGAATAAGGATCGGCTAACTCCGTGCCAGCAGCCGCGGTAA
TACGGAGGATCCGAGCGTTATCCGGATTTATTGGGTTTAAAGGGAGCGTAGGCGGA
CGCTTAAGTCAGTTGTGAAAGTTTGCGGCTCAACCGTAAAATTGCAGTTGATACTGG
GTGTCTTGAGTACAGTAGAGGCAGGCGGAATTCGTGGTGTAGCGGTGAAATGCTTA
GATATCACGAAGAACTCCGATTGCGAAGGCAGCCTGCTGGACTGTAACTGACGCTG
ATGCTCGAAAGTGTGGGTATCAAACAGGATTAGATACCCTGGTAGTCCACACCAGT
AAACGATGAATACTCGCTGTTTGCGATATACAGTAAGCGGCCAAGCGAAAGCGTTA
AGTATTCCACCTGGGGAGTACGCCGGCAACGGTGAAACTCAAAGGAATTGACGGG
GGCCCGCACAAGCGGAGGAACATGTGGTTTAATTCGATGATACGCGAGGAACCTTA
CCCGGGCTTGAATTGCAACTGAATGATGTGGAGACATGTCAGCCGCAAGGCAGTTG
TGAAGGTGCTGCATGGTTGTCGTCAGCTCGTGCCGTGAGGTGTCGGCTTAAGTGCC
ATAACGAGCGCAACCCTTATCGATAGTTACCATCAGGTGATGCTGGGGACTCTGTC
GAGACTGCCGTCGTAAGATGTGAGGAAGGTGGGGATGACGTCAAATCAGCACGGC
CCTTACGTCCGGGGCTACACACGTGTTACAATGGGGGGTACAGAAGGCAGCTACAC
GGCGACGTGATGCTAATCCCGAAAGCCTCTCTCAGTTCGGATTGGAGTCTGCAACC
CGACTCCATGAAGCTGGATTCGCTAGTAATCGCGCATCAGCCACGGCGCGGTGAAT
ACGTTCCCGGGCCTTGTACACACCGCCCGTCAAGCCATGAAAGCCGGGGGTACCT
GAAGTGCGTAACCGCAAGGAG
SEQ ID NO: 5 2B1_Subdoligranulum sp. 4_3_54A2FAA_NZ-ACWW00000000
GACGAACGCTGGCGGCGCGCCTAACACATGCAAGTCGAACGGAGCTGTTTTCTCTG
AAGTTTTCGGATGGAAGAGAGTTCAGCTTAGTGGCGAACGGGTGAGTAACACGTGA
GCAACCTGCCTTTCAGTGGGGGACAACATTTGGAAACGAATGCTAATACCGCATAA
GACCACAGTGTCGCATGGCACAGGGGTCAAAGGATTTATCCGCTGAAAGATGGGCT
CGCGTCCGATTAGCTAGATGGTGAGGTAACGGCCCACCATGGCGACGATCGGTAG
CCGGACTGAGAGGTTGAACGGCCACATTGGGACTGAGACACGGCCCAGACTCCTA
CGGGAGGCAGCAGTGGGGAATATTGCACAATGGGGGAAACCCTGATGCAGCGACG
CCGCGTGGAGGAAGAAGGTCTTCGGATTGTAAACTCCTGTCCCAGGGGACGATAAT
GACGGTACCCTGGGAGGAAGCACCGGCTAACTACGTGCCAGCAGCCGCGGTAAAA
CGTAGGGTGCAAGCGTTGTCCGGAATTACTGGGTGTAAAGGGAGCGCAGGCGGAT
TGGCAAGTTGGGAGTGAAATCTATGGGCTCAACCCATAAATTGCTTTCAAAACTGTC
AGTCTTGAGTGGTGTAGAGGTAGGCGGAATTCCCGGTGTAGCGGTGGAATGCGTA
GATATCGGGAGGAACACCAGTGGCGAAGGCGGCCTACTGGGCACTAACTGACGCT
GAGGCTCGAAAGCATGGGTAGCAAACAGGATTAGATACCCTGGTAGTCCATGCCGT
AAACGATGATTACTAGGTGTGGGAGGATTGACCCCTTCCGTGCCGCAGTTAACACA
ATAAGTAATCCACCTGGGGAGTACGACCGCAAGGTTGAAACTCAAAGGAATTGACG
GGGGCCCGCACAAGCAGTGGAGTATGTGGTTTAATTCGAAGCAACGCGAAGAACCT
TACCAGGTCTTGACATCGGATGCATACCTAAGAGATTAGGGAAGTCCTTCGGGACA
TCCAGACAGGTGGTGCATGGTTGTCGTCAGCTCGTGTCGTGAGATGTTGGGTTAAG
TCCCGCAACGAGCGCAACCCTTATCGTTAGTTACTACGCAAGAGGACTCTAGCGAG
ACTGCCGTTGACAAAACGGAGGAAGGTGGGGATGACGTCAAATCATCATGCCCTTT
ATGACCTGGGCTACACACGTACTACAATGGCTATTAACAGAGAGAAGCGATACCGC
GAGGTGGAGCAAACCTCACAAAAATAGTCTCAGTTCGGATCGCAGGCTGCAACCCG
CCTGCGTGAAGCCGGAATTGCTAGTAATCGCGGATCAGCATGCCGCGGTGAATACG
TTCCCGGGCCTTGTACACACCGCCCGTCACACCATGAGAGCCGGGGGGACCCGAA
GTCGGTAGTCTAACCGC
SEQ ID NO: 6 2A6_Paraprevotella xylaniphila_AB 331897
GATGAACGCTAGCTACAGGCTTAACACATGCAAGTCGAGGGGCAGCATGAACTTAG
CTTGCTAAGTTTGATGGCGACCGGCGCACGGGTGAGTAACGCGTATCCAACCTGCC
CTTTACCCGGGGATAGCCTTCTGAAAAGGAAGTTTAATACCCGATGAATTCGTTTAG
TCGCATGGCTNGATGAATAAAGATTAATTGGTAAAGGATGGGGATGCGTCCCATTA
GCTTGTTGGCGGGGTAACGGCCCACCAAGGCGACGATGGGTAGGGGTTCTGAGAG
GAAGGTCCCCCACATTGGAACTGAGACACGGTCCAAACTCCTACGGGAGGCAGCA
GTGAGGAATATTGGTCAATGGGCGCGAGCCTGAACCAGCCAAGTAGCGTGGAGGA
CGACGGCCCTACGGGTTGTAAACTCCTTTTATAAGGGGATAAAGTTGGCCATGTAT
GGCCATTTGCAGGTACCTTATGAATAAGCATCGGCTAATTCCGTGCCAGCAGCCGC
GGTAATACGGAAGATGCGAGCGTTATCCGGATTTATTGGGTTTAAAGGGAGCGTAG
GCGGGCTGTCAAGTCAGCGGTCAAATGGCGCGGCTCAACCGCGTTCCGCCGTTGA
AACTGGCAGCCTTGAGTATGCACAGGGTACATGGAATTCGTGGTGTAGCGGTGAAA
TGCTTAGATATCACGAGGAACTCCGATCGCGCAGGCATTGTACCGGGGCATTACTG
ACGCTGAGGCTCGAAGGTGCGGGTATCAAACAGGATTAGATACCCTGGTAGTCCGC
ACAGTAAACGATGAATGCCCGCTGTCGGCGACATAGTGTCGGCGGCCAAGCGAAA
GCGTTAAGCATTCCACCTGGGGAGTACGCCGGCAACGGTGAAACTCAAAGGAATTG
ACGGGGGCCCGCACAAGCGGAGGAACATGTGGTTTAATTCGATGATACGCGAGGA
ACCTTACCCGGGCTTGAATCGCAGGTGCATGGGCCGGAGACGGCCCTTTCCTTCG
GGACTCCTGCGAAGGTGCTGCATGGTTGTCGTCAGCTCGTGCCGTGAGGTGTCGG
CTTAAGTGCCATAACGAGCGCAACCCCCCTCCCCAGTTGCCACCGGGTAATGCCGG
GCACTTTGGGGACACTGCCACCGCAAGGTGCGAGGAAGGTGGGGATGACGTCAAA
TCAGCACGGCCCTTACGTCCGGGGCGACACACGTGTTACAATGGGGGGTACAGAG
GGCCGCTGCCCGGTGACGGTTGGCCAATCCCTAAAACCCCTCTCAGTTCGGACTG
GAGTCTGCAACCCGACTCCACGAAGCTGGATTCGCTAGTAATCGCGCATCAGCCAT
GGCGCGGTGAATACGTTCCCGGGCCTTGTACACACCGCCCGTCAAGCCATGAAAG
CCGGGGGTGCCTGAAGTCCGTNNCCGCGA
SEQ ID NO: 7 2F11_Parabacteroides johnsonii_AB261128
GATGAACGCTAGCGACAGGCTTAACACATGCAAGTCGAGGGGCAGCATGGTAAGTA
GCAATACTTATTGATGGCGACCGGCGCACGGGTGAGTAACGCGTATGCAACTTACC
TATCAGAGGGGGATAGCCCGGCGAAAGTCGGATTAATACTCCATAAAACAGGGGTT
CCGCATGGGACTATTTGTTAAAGATTCATCGCTGATAGATAGGCATGCGTTCCATTA
GGCAGTTGGCGGGGTAACGGCCCACCAAACCGACGATGGATAGGGGTTCTGAGAG
GAAGGTCCCCCACATTGGTACTGAGACACGGACCAAACTCCTACGGGAGGCAGCA
GTGAGGAATATTGGTCAATGGCCGAGAGGCTGAACCAGCCAAGTCGCGTGAAGGA
TGAAGGATCTATGGTTTGTAAACTTCTTTTATAGGGGAATAAAGTGTGGGACGTGTT
CCATTTTGTATGTACCCTATGAATAAGCATCGGCTAACTCCGTGCCAGCAGCCGCG
GTAATACGGAGGATGCGAGCGTTATCCGGATTTATTGGGTTTAAAGGGTGCGTAGG
TGGTAATTTAAGTCAGCGGTGAAAGTTTGTGGCTCAACCATAAAATTGCCGTTGAAA
CTGGGTTACTTGAGTGTGTTTGAGGTAGGCGGAATGCGTGGTGTAGCGGTGAAATG
CATAGATATCACGCAGAACTCCAATTGCGAAGGCAGCTTACTAAACCATAACTGACA
CTGAAGCACGAAAGCGTGGGTATCAAACAGGATTAGATACCCTGGTAGTCCACGCA
GTAAACGATGATTACTAGGAGTTTGCGATACACAGTAAGCTCTACAGCGAAAGCGTT
AAGTAATCCACCTGGGGAGTACGCCGGCAACGGTGAAACTCAAAGGAATTGACGG
GGGCCCGCACAAGCGGAGGAACATGTGGTTTAATTCGATGATACGCGAGGAACCTT
ACCCGGGTTTGAACGTAGTCAGACCGACCTTGAAAGAGGTTTTCTAGCAATAGCTG
ATTACGAGGTGCTGCATGGTTGTCGTCAGCTCGTGCCGTGAGGTGTCGGCTTAAGT
GCCATAACGAGCGCAACCCTTATCACTAGTTACTAACAGGTTAAGCTGAGGACTCT
GGTGAGACTGCCAGCGTAAGCTGTGAGGAAGGTGGGGATGACGTCAAATCAGCAC
GGCCCTTACATCCGGGGCGACACACGTGTTACAATGGCATGGACAAAGGGCAGCT
ACCTGGCGACAGGATGCTAATCTCTAAACCATGTCTCAGTTCGGATCGGAGTCTGC
AACTCGACTCCGTGAAGCTGGATTCGCTAGTAATCGCGCATCAGCCATGGCGCGGT
GAATACGTTCCCGGGCCTTGTACACACCGCCCGTCAAGCCATGGGAGCCGGGGGT
ACCTGAAGTCCGTAACCGCAA
SEQ ID NO: 8 1E7_Alistipes sp. JC136_NZ-CAEG00000000
GATGAACGCTAGCGGCAGGCCTAACACATGCAAGTCGAGGGGCAGCGGGATTGAA
GCTTGCTTCAGTTGCCGGCGACCGGCGCACGGGTGCGTAACGCGTATGCAACCTA
CCCATAACAGGGGGATAACACTGAGAAATCGGTACTAATATCCCATAACATCAAGAG
GGGCATCCCTTTTGGTTGAAAACTCCGGTGGTTATGGATGGGCATGCGTTGTATTA
GCTAGTTGGTGAGGTAACGGCTCACCAAGGCGACGATACATAGGGGGACTGAGAG
GTTAACCCCCCACATTGGTACTGAGACACGGACCAAACTCCTACGGGAGGCAGCAG
TGAGGAATATTGGTCAATGGACGCAAGTCTGAACCAGCCATGCCGCGTGCAGGATG
ACGGCTCTATGAGTTGTAAACTGCTTTTGTACGAGGGTAAACCCGGATACGTGTATC
CGGCTGAAAGTATCGTACGAATAAGGATCGGCTAACTCCGTGCCAGCAGCCGCGGT
AATACGGAGGATTCAAGCGTTATCCGGATTTATTGGGTTTAAAGGGTGCGTAGGCG
GTTTGATAAGTTAGAGGTGAAATACCGGTGCTTAACACCGGAACTGCCTCTAATACT
GTTGAGCTAGAGAGTAGTTGCGGTAGGCGGAATGTATGGTGTAGCGGTGAAATGCT
TAGAGATCATACAGAACACCGATTGCNGAAGGCAGCTTACCAAACTATATCTGACGT
TNGAGGCACGAAAGCGTGGGGGAGCAAACAGGATTAGATACCCTGGTAGTCCACG
CAGTAAACGATGATAACTCGCTGTCGGCGATACACAGTCGGTGGCTAAGCGAAAGC
GATAAGTTATCCACCTGGGGAGTACGTTCGCAAGAATGAAACTCAAAGGAATTGAC
GGGGGCCCGCACAAGCGGAGGAACATGTGGTTTAATTCGATGATACGCGAGGAAC
CTTACCCGGGCTTGAAAGTTACTGACGATTCTGGAAACAGGATTTCCCTTCGGGGC
AGGAAACTAGGTGCTGCATGGTTGTCGTCAGCTCGTGCCGTGAGGTGTCGGGTTAA
GTCCCATAACGAGCGCAACCCCTACCGTTAGTTGCCATCAGGTCAAGCTGGGCACT
CTGGCGGGACTGCCGGTGTAAGCCGAGAGGAAGGTGGGGATGACGTCAAATCAGC
ACGGCCCTTACGTCCGGGGCTACACACGTGTTACAATGGTAGGTACAGAGGGCAG
CTACCCAGTGATGGGATGCGAATCTCGAAAGCCTATCTCAGTTCGGATTGGAGGCT
GAAACCCGCCTCCATGAAGTTGGATTCGCTAGTAATCGCGCATCAGCCATGGCGCG
GTGAATACGTTCCCGGGCCTTGTACACACCGCCCGTCAAGCCATGGAAGCTGGGG
GTGCCTGAAGTTCGTGAC
SEQ ID NO: 9 1H9_Parabacteroides gordonii_AB470343
GATGAACGCTAGCGACAGGCTTAACACATGCAAGTCGAGGGGCAGCAGGAAGTAG
CAATACTTTGCTGGCGACCGGCGCACGGGTGAGTAACGCGTATGCAACCTACCTAT
CAGAGGGGGATAACCCGGCGAAAGTCGGACTAATACCGCATAAAACAGGGGTCCC
GCATGGGAATATTTGTTAAAGATTTATTGCTGATAGATGGGCATGCGTTCCATTAGA
TAGTTGGTGAGGTAACGGCTCACCAAGTCTTCGATGGATAGGGGTTCTGAGAGGAA
GGTCCCCCACACTGGTACTGAGACACGGACCAGACTCCTACGGGAGGCAGCAGTG
AGGAATATTGGTCAATGGGCGAGAGCCTGAACCAGCCAAGTCGCGTGAAGGATGAA
GGATCTATGGTTCGTAAACTTCTTTTATAGGGGAATAAAGTGCAGGACGTGTCCTGT
TTTGTATGTACCCTATGAATAAGGATCGGCTAACTCCGTGCCAGCAGCCGCGGTAA
TACGGAGGATCCGAGCGTTATCCGGATTTATTGGGTTTAAAGGGTGCGTAGGTGGC
TTTTTAAGTCAGCGGTGAAAGTTTGTGGCTCAACCATAAAATTGCCGTTGAAACTGG
AGGGCTTGAGTATATTTGAGGTAGGCGGAATGCGTGGTGTAGCGGTGAAATGCATA
GATATCACGCAGAACTCCAATTGCGAAGGCAGCTTACTAAACTATAACTGACACTGA
AGCACGAAAGCGTGGGGATCAAACAGGATTAGATACCCTGGTAGTCCACGCAGTAA
ACGATGATTACTAGGAGTTTGCGATACACAGTAAGCTCTACAGCGAAAGCGTTAAGT
AATCCACCTGGGGAGTACGCCGGCAACGGTGAAACTCAAAGGAATTGACGGGGGC
CCGCACAAGCGGAGGAACATGTGGTTTAATTCGATGATACGCGAGGAACCTTACCC
GGGTTTGAACGTAAGTTGACCGGAGTGGAAACACTCTTTCTAGCAATAGCAATTTAC
GAGGTGCTGCATGGTTGTCGTCAGCTCGTGCCGTGAGGTGTCGGCTTAAGTGCCAT
AACGAGCGCAACCCTTATCTTTAGTTACTAACAGGTCGAGCTGAGGACTCTAAAGA
GACTGCCAGCGTAAGCTGTGAGGAAGGTGGGGATGACGTCAAATCAGCACGGCCC
TTACATCCGGGGCGACACACGTGTTACAATGGTGGGGACAAAGGGCAGCTACCTG
GCGACAGGATGCTAATCTCCAAACCCCATCTCAGTTCGGATCGAAGTCTGCAACCC
GACTTCGTGAAGCTGGATTCGCTAGTAATCGCGCATCAGCCATGGCGCGGTGAATA
CGTTCCCGGGCCTTGTACACACCGCCCGTCAAGCCATGGGAGTTGGGGGTACCTA
AAGTCCGTNACCGCAAG
SEQ ID NO: 10 1C1_Eubacterium limosum_AB595134
GACGAACGCTGGCGGTATGCTTAACACATGCAAGTCGAACGAGAAGGTTTTGATGG
ATCCTTCGGGTGACATTAGAACTGGAAAGTGGCGAACGGGTGAGTAACGCGTGGGT
AACCTGCCCTATGGAAAGGAATAGCCTCGGGAAACTGGGAGTAAAGCCTTATATTA
TGGTTTTGTCGCATGGCAAGATCATGAAAACTCCGGTGCCATAGGATGGACCCGCG
TCCCATTAGCTAGTTGGTGAGATAACAGCCCACCAAGGCGACGATGGGTAACCGGT
CTGAGAGGGCGAACGGTCACACTGGAACTGAGACACGGTCCAGACTCCTACGGGA
GGCAGCAGTGGGGAATATTGCGCAATGGGGGCAACCCTGACGCAGCAATACCGCG
TGAGTGAAGAAGGTTTTCGGATCGTAAAGCTCTGTTATTGGGGAAGAAGAATGACG
GTACCCAATGAGGAAGTCCCGGCTAACTACGTGCCAGCAGCCGCGGTAATACGTAG
GGGACAAGCGTTGTCCGGAATGACTGGGCGTAAAGGGCGCGTAGGCGGTCTATTA
AGTCTGATGTGAAAGGTACCGGCTCAACCGGTGAAGTGCATTGGAAACTGGTAGAC
TTGAGTATTGGAGAGGCAAGTGGAATTCCTAGTGTAGCGGTGAAATGCGTAGATAT
TAGGAGGAACACCAGTGGCGAAGGCGGCTTGCTGGACAAATACTGACGCTGAGGT
GCGAAAGCGTGGGGAGCGAACAGGATTAGATACCCTGGTAGTCCACGCCGTAAAC
GATGAATGCTAGGTGTTGGGGAAACTCAGTGCCGCAGTTAACACAATAAGCATTCC
GCCTGGGGAGTACGACCGCAAGGTTGAAACTCAAAGGAATTGACGGGGACCCGCA
CAAGCAGCGGAGCATGTGGTTTAATTCGAAGCAACGCGAAGAACCTTACCAGGTCT
TGACATCCTCTGACGAGCCTAGAGATAGGAAGTTTCCTTCGGGAACAGAGAGACAG
GTGGTGCATGGTTGTCGTCAGCTCGTGTCGTGAGATGTTGGGTTAAGTCCCGCAAC
GAGCGCAACCCCTGCCTTTAGTTGCCAGCATTAAGTTGGGCACTCTAGAGGGACTG
CCGTAGACAATACGGAGGAAGGTGGGGACGACGTCAAATCATCATGCCCCTTATGA
CCTGGGCTACACACGTGCTACAATGGTCTGAACAGAGGGCCGCGAAGCCGCGAGG
TGAAGCAAATCCCTTAAAACAGATCCCAGTTCGGATTGCAGGCTGCAACTCGCCTG
CATGAAGTTGGAGTTGCTAGTAATCGCGGATCAGAATGCCGCGGTGAATGCGTTCC
CGGGTCTTGTACACACCGCCCGTCACACCACGAGAGTTGGCAACACCCGAAGCCT
GTGAGAGAACCGTAAGGACTCAGCAGT
SEQ ID NO: 11 2G9_Parabacteroides distasonis_HE974920
GATGAACGCTAGCGACAGGCTTAACACATGCAAGTCGAGGGGCAGCACAGGTAGC
AATACCGGGTGGCGACCGGCGCACGGGTGAGTAACGCGTATGCAACTTGCCTATC
AGAGGGGGATAACCCGGCGAAAGTCGGACTAATACCGCATGAAGCAGGGGCCCCG
CATGGGGATATTTGCTAAAGATTCATCGCTGATAGATAGGCATGCGTTCCATTAGGC
AGTTGGCGGGGTAACGGCCCACCAAACCGACGATGGATAGGGGTTCTGAGAGGAA
GGTCCCCCACATTGGTACTGAGACACGGACCAAACTCCTACGGGAGGCAGCAGTG
AGGAATATTGGTCAATGGCCGAGAGGCTGAACCAGCCAAGTCGCGTGAGGGATGA
AGGTTCTATGGATCGTAAACCTCTTTTATAAGGGAATAAAGTGCGGGACGTGTCCC
GTTTTGTATGTACCTTATGAATAAGGATCGGCTAACTCCGTGCCAGCAGCCGCGGT
AATACGGAGGATCCGAGCGTTATCCGGATTTATTGGGTTTAAAGGGTGCGTAGGCG
GCCTTTTAAGTCAGCGGTGAAAGTCTGTGGCTCAACCATAGAATTGCCGTTGAAACT
GGGGGGCTTGAGTATGTTTGAGGCAGGCGGAATGCGTGGTGTAGCGGTGAAATGC
ATAGATATCACGCAGAACCCCGATTGCGAAGGCAGCCTGCCAAGCCATTACTGACG
CTGATGCACGAAAGCGTGGGGATCAAACAGGATTAGATACCCTGGTAGTCCACGCA
GTAAACGATGATCACTAGCTGTTTGCGATACACTGTAAGCGGCACAGCGAAAGCGT
TAAGTGATCCACCTGGGGAGTACGCCGGCAACGGTGAAACTCAAAGGAATTGACGG
GGGCCCGCACAAGCGGAGGAACATGTGGTTTAATTCGATGATACGCGAGGAACCTT
ACCCGGGTTTGAACGCATTCGGACCGAGGTGGAAACACCTTTTCTAGCAATAGCCG
TTTGCGAGGTGCTGCATGGTTGTCGTCAGCTCGTGCCGTGAGGTGTCGGCTTAAGT
GCCATAACGAGCGCAACCCTTGCCACTAGTTACTAACAGGTAAAGCTGAGGACTCT
GGTGGGACTGCCAGCGTAAGCTGCGAGGAAGGCGGGGATGACGTCAAATCAGCAC
GGCCCTTACATCCGGGGCGACACACGTGTTACAATGGCGTGGACAAAGGGAAGCC
ACCTGGCGACAGGGAGCGAATCCCCAAACCACGTCTCAGTTCGGATCGGAGTCTG
CAACCCGACTCCGTGAAGCTGGATTCGCTAGTAATCGCGCATCAGCCATGGCGCG
GTGAATACGTTCCCGGGCCTTGTACACACCGCCCGTCAAGCCATGGGAGCCNGGG
GTACCTGAAGTCCGTAACCGCGA
SEQ ID NO: 36-Phascolarctobacterium faecium
ATTGGAGAGTTTGATCCTGGCTCAGGACGAACGCTGGCGGCGTGCCTAACACATGC
AAGTCGAACGGAGAATTTTATTTCGGTAGAATTCTTAGTGGCGAACGGGTGAGTAAC
GCGTAGGCAACCTACCCTTTAGACGGGGACAACATTCCGAAAGGAGTGCTAATACC
GGATGTGATCATCTTGCCGCATGGCAGGATGAAGAAAGATGGCCTCTACAAGTAAG
CTATCGCTAAAGGATGGGCCTGCGTCTGATTAGCTAGTTGGTAGTGTAACGGACTA
CCAAGGCGATGATCAGTAGCCGGTCTGAGAGGATGAACGGCCACATTGGGACTGA
GACACGGCCCAAACTCCTACGGGAGGCAGCAGTGGGGAATCTTCCGCAATGGACG
AAAGTCTGACGGAGCAACGCCGCGTGAGTGATGAAGGATTTCGGTCTGTAAAGCTC
TGTTGTTTATGACGAACGTGCAGTGTGTGAACAATGCATTGCAATGACGGTAGTAAA
CGAGGAAGCCACGGCTAACTACGTGCCAGCAGCCGCGGTAATACGTAGGTGGCGA
GCGTTGTCCGGAATTATTGGGCGTAAAGAGCATGTAGGCGGCTTAATAAGTCGAGC
GTGAAAATGCGGGGCTCAACCCCGTATGGCGCTGGAAACTGTTAGGCTTGAGTGCA
GGAGAGGAAAGGGGAATTCCCAGTGTAGCGGTGAAATGCGTAGATATTGGGAGGA
ACACCAGTGGCGAAGGCGCCTTTCTGGACTGTGTCTGACGCTGAGATGCGAAAGC
CAGGGTAGCGAACGGGATTAGATACCCCGGTAGTCCTGGCCGTAAACGATGGGTA
CTAGGTGTAGGAGGTATCGACCCCTTCTGTGCCGGAGTTAACGCAATAAGTACCCC
GCCTGGGGAGTACGGCCGCAAGGTTGAAACTCAAAGGAATTGACGGGGGCCCGCA
CAAGCGGTGGAGTATGTGGTTTAATTCGACGCAACGCGAAGAACCTTACCAAGGCT
TGACATTGATTGAACGCTCTAGAGATAGAGATTTCCCTTCGGGGACAAGAAAACAG
GTGGTGCATGGCTGTCGTCAGCTCGTGTCGTGAGATGTTGGGTTAAGTCCCGCAAC
GAGCGCAACCCCTATCCTATGTTACCAGCAAGTAAAGTTGGGGACTCATGGGAGAC
TGCCAGGGACAACCTGGAGGAAGGCGGGGATGACGTCAAGTCATCATGCCCCTTA
TGTCTTGGGCTACACACGTACTACAATGGTCGGAAACAGAGGGAAGCGAAGCCGC
GAGGCAGAGCAAACCCCAGAAACCCGATCTCAGTTCGGATCGCAGGCTGCAACCC
GCCTGCGTGAAGTCGGAATCGCTAGTAATCGCAGGTCAGCATACTGCGGTGAATAC
GTTCCCGGGCCTTGTACACACCGCCCGTCACACCACGAAAGTTGGTAACACCCGAA
GCCGGTGAGGTAACCTATTAGGAGCCAGCCGTCTAAGGTGGGGCCGATGATTGGG
GTGAAGTCGTAACAAGGTAGCCGTATCGGAAGGTGCGGCTGGATCACCTCCTTT
SEQ ID NO: 37-Fusobacterium varium
CGAAGAGTTTGATCCTGGCTCAGGATGAACGCTGACAGAATGCTTAACACATGCAA
GTCTACTTGATCCTTCGGGTGAAGGTGGCGGACGGGTGAGTAACGCGTAAAGAACT
TGCCTTACAGACTGGGACAACATTTGGAAACGAATGCTAATACCGGATATTATGATT
GGGTCGCATGATCTGATTATGAAAGCTATATGCGCTGTGAGAGAGCTTTGCGTCCC
ATTAGTTAGTTGGTGAGGTAACGGCTCACCAAGACGATGATGGGTAGCCGGCCTGA
GAGGGTGAACGGCCACAAGGGGACTGAGACACGGCCCTTACTCCTACGGGAGGCA
GCAGTGGGGAATATTGGACAATGGACCAAAAGTCTGATCCAGCAATTCTGTGTGCA
CGAAGAAGTTTTTCGGAATGTAAAGTGCTTTCAGTTGGGAAGAAGTCAGTGACGGT
ACCAACAGAAGAAGCGACGGCTAAATACGTGCCAGCAGCCGCGGTAATACGTATGT
CGCAAGCGTTATCCGGATTTATTGGGCGTAAAGCGCGTCTAGGCGGCTTAGTAAGT
CTGATGTGAAAATGCGGGGCTCAACCCCGTATTGCGTTGGAAACTGCTAAACTAGA
GTACTGGAGAGGTAGGCGGAACTACAAGTGTAGAGGTGAAATTCGTAGATATTTGT
AGGAATGCCGATGGGGAAGCCAGCCTACTGGACAGATACTGACGCTAAAGCGCGA
AAGCGTGGGTAGCAAACAGGATTAGATACCCTGGTAGTCCACGCCGTAAACGATGA
TTACTAGGTGTTGGGGGTCGAACCTCAGCGCCCAAGCTAACGCGATAAGTAATCCG
CCTGGGGAGTACGTACGCAAGTATGAAACTCAAAGGAATTGACGGGGACCCGCACA
AGCGGTGGAGCATGTGGTTTAATTCGACGCAACGCGAGGAACCTTACCAGCGTTTG
ACATCCCAAGAAGTTAACAGAGATGTTTTCGTGCCTCTTCGGAGGAACTTGGTGACA
GGTGGTGCATGGCTGTCGTCAGCTCGTGTCGTGAGATGTTGGGTTAAGTCCCGCAA
CGAGCGCAACCCCTTTCGTATGTTACCATCATTAAGTTGGGGACTCATGCGAGACT
GCCTGCGATGAGCAGGAGGAAGGTGGGGATGACGTCAAGTCATCATGCCCCTTATA
CGCTGGGCTACACACGTGCTACAATGGGTAGTACAGAGAGCTGCAAACCTGCGAG
GGTAAGCTAATCTCATAAAACTATTCTTAGTTCGGATTGTACTCTGCAACTCGAGTA
CATGAAGTTGGAATCGCTAGTAATCGCAAATCAGCTATGTTGCGGTGAATACGTTCT
CGGGTCTTGTACACACCGCCCGTCACACCACGAGAGTTGGTTGCACCTGAAGTAAC
AGGCCTAACCGTAAGGAGGGATGTTCCGAGGGTGTGATTAGCGATTGGGGTGAAG
TCGTAACAAGGTATCCGTACGGGAACGTGCGGATGGATCACCTCCTT
SEQ ID NO: 38-Bacteroides dorei
ATGAAGAGTTTGATCCTGGCTCAGGATGAACGCTAGCTACAGGCTTAACACATGCA
AGTCGAGGGGCAGCATGGTCTTAGCTTGCTAAGGCTGATGGCGACCGGCGCACGG
GTGAGTAACACGTATCCAACCTGCCGTCTACTCTTGGCCAGCCTTCTGAAAGGAAG
ATTAATCCAGGATGGGATCATGAGTTCACATGTCCGCATGATTAAAGGTATTTTCCG
GTAGACGATGGGGATGCGTTCCATTAGATAGTAGGCGGGGTAACGGCCCACCTAGT
CAACGATGGATAGGGGTTCTGAGAGGAAGGTCCCCCACATTGGAACTGAGACACG
GTCCAAACTCCTACGGGAGGCAGCAGTGAGGAATATTGGTCAATGGGCGATGGCCT
GAACCAGCCAAGTAGCGTGAAGGATGACTGCCCTATGGGTTGTAAACTTCTTTTATA
AAGGAATAAAGTCGGGTATGCATACCCGTTTGCATGTACTTTATGAATAAGGATCGG
CTAACTCCGTGCCAGCAGCCGCGGTAATACGGAGGATCCGAGCGTTATCCGGATTT
ATTGGGTTTAAAGGGAGCGTAGATGGATGTTTAAGTCAGTTGTGAAAGTTTGCGGCT
CAACCGTAAAATTGCAGTTGATACTGGATGTCTTGAGTGCAGTTGAGGCAGGCGGA
ATTCGTGGTGTAGCGGTGAAATGCTTAGATATCACGAAGAACTCCGATTGCGAAGG
CAGCCTGCTAAGCTGCAACTGACATTGAGGCTCGAAAGTGTGGGTATCAAACAGGA
TTAGATACCCTGGTAGTCCACACGGTAAACGATGAATACTCGCTGTTTGCGATATAC
GGCAAGCGGCCAAGCGAAAGCGTTAAGTATTCCACCTGGGGAGTACGCCGGCAAC
GGTGAAACTCAAAGGAATTGACGGGGGCCCGCACAAGCGGAGGAACATGTGGTTT
AATTCGATGATACGCGAGGAACCTTACCCGGGCTTAAATTGCACTCGAATGATCCG
GAAACGGTTCAGCTAGCAATAGCGAGTGTGAAGGTGCTGCATGGTTGTCGTCAGCT
CGTGCCGTGAGGTGTCGGCTTAAGTGCCATAACGAGCGCAACCCTTGTTGTCAGTT
ACTAACAGGTGATGCTGAGGACTCTGACAAGACTGCCATCGTAAGATGTGAGGAAG
GTGGGGATGACGTCAAATCAGCACGGCCCTTACGTCCGGGGCTACACACGTGTTAC
AATGGGGGGTACAGAGGGCCGCTACCACGCGAGTGGATGCCAATCCCTAAAACCC
CTCTCAGTTCGGACTGGAGTCTGCAACCCGACTCCACGAAGCTGGATTCGCTAGTA
ATCGCGCATCAGCCACGGCGCGGTGAATACGTTCCCGGGCCTTGTACACACCGCC
CGTCAAGCCATGGGAGCCGGGGGTACCTGAAGTGCGTAACCGCGAGGATCGCCCT
AGGGTAAAACTGGTGACTGGGGCTAAGTCGTAACAAGGTAGCCGTACCGGAAGGT
GCGGCTGGAACACCTCCTT
SEQ ID NO: 39 Bacteroides uniformis
ATGAAGAGTTTGATCCTGGCTCAGGATGAACGCTAGCTACAGGCTTAACACATGCA
AGTCGAGGGGCAGCATGAACTTAGCTTGCTAAGTTTGATGGCGACCGGCGCACGG
GTGAGTAACACGTATCCAACCTGCCGATGACTCGGGGATAGCCTTTCGAAAGAAAG
ATTAATACCCGATGGCATAGTTCTTCCGCATGGTGGAACTATTAAAGAATTTCGGTC
ATCGATGGGGATGCGTTCCATTAGGTTGTTGGCGGGGTAACGGCCCACCAAGCCTT
CGATGGATAGGGGTTCTGAGAGGAAGGTCCCCCACATTGGAACTGAGACACGGTC
CAAACTCCTACGGGAGGCAGCAGTGAGGAATATTGGTCAATGGACGAGAGTCTGAA
CCAGCCAAGTAGCGTGAAGGATGACTGCCCTATGGGTTGTAAACTTCTTTTATACG
GGAATAAAGTGAGGCACGTGTGCCTTTTTGTATGTACCGTATGAATAAGGATCGGCT
AACTCCGTGCCAGCAGCCGCGGTAATACGGAGGATCCGAGCGTTATCCGGATTTAT
TGGGTTTAAAGGGAGCGTAGGCGGACGCTTAAGTCAGTTGTGAAAGTTTGCGGCTC
AACCGTAAAATTGCAGTTGATACTGGGTGTCTTGAGTACAGTAGAGGCAGGCGGAA
TTCGTGGTGTAGCGGTGAAATGCTTAGATATCACGAAGAACTCCGATTGCGAAGGC
AGCCTGCTGGACTGTAACTGACGCTGATGCTCGAAAGTGTGGGTATCAAACAGGAT
TAGATACCCTGGTAGTCCACACAGTAAACGATGAATACTCGCTGTTTGCGATATACA
GTAAGCGGCCAAGCGAAAGCGTTAAGTATTCCACCTGGGGAGTACGCCGGCAACG
GTGAAACTCAAAGGAATTGACGGGGGCCCGCACAAGCGGAGGAACATGTGGTTTAA
TTCGATGATACGCGAGGAACCTTACCCGGGCTTGAATTGCAACTGAATGATGTGGA
GACATGTCAGCCGCAAGGCAGTTGTGAAGGTGCTGCATGGTTGTCGTCAGCTCGTG
CCGTGAGGTGTCGGCTTAAGTGCCATAACGAGCGCAACCCTTATCGATAGTTACCA
TCAGGTGATGCTGGGGACTCTGTCGAGACTGCCGTCGTAAGATGTGAGGAAGGTG
GGGATGACGTCAAATCAGCACGGCCCTTACGTCCGGGGCTACACACGTGTTACAAT
GGGGGGTACAGAAGGCAGCTACACGGCGACGTGATGCTAATCCCGAAAGCCTCTC
TCAGTTCGGATTGGAGTCTGCAACCCGACTCCATGAAGCTGGATTCGCTAGTAATC
GCGCATCAGCCACGGCGCGGTGAATACGTTCCCGGGCCTTGTACACACCGCCCGT
CAAGCCATGAAAGCCGGGGGTACCTGAAGTGCGTAACCGCAAGGAGCGCCCTAGG
GTAAAACTGGTGATTGGGGCTAAGTCGTAACAAGGTAGCCGTACCGGAAGGTGCG
GCTGGAACACCTCCTT
SEQ ID NO: 40 Gemminger formicilis
AATGAAGAGTTTGATCCTGGCTCAGGACGAACGCTGGCGGCGCGCCTAACACATGC
AAGTCGAACGGAGCTGTTTTCTCTGAAGTTTTCGGATGGAAGAGAGTTCAGCTTAGT
GGCGAACGGGTGAGTAACACGTGAGCAACCTGCCTTTCAGTGGGGGACAACATTTG
GAAACGAATGCTAATACCGCATAAGACCACAGTGTCGCATGGCACAGGGGTCAAAG
GATTTATCCGCTGAAAGATGGGCTCGCGTCCGATTAGCTAGATGGTGAGGTAACGG
CCCACCATGGCGACGATCGGTAGCCGGACTGAGAGGTTGAACGGCCACATTGGGA
CTGAGACACGGCCCAGACTCCTACGGGAGGCAGCAGTGGGGAATATTGCACAATG
GGGGAAACCCTGATGCAGCGACGCCGCGTGGAGGAAGAAGGTCTTCGGATTGTAA
ACTCCTGTCCCAGGGGACGATAATGACGGTACCCTGGGAGGAAGCACCGGCTAAC
TACGTGCCAGCAGCCGCGGTAAAACGTAGGGTGCAAGCGTTGTCCGGAATTACTG
GGTGTAAAGGGAGCGCAGGCGGATTGGCAAGTTGGGAGTGAAATCTATGGGCTCA
ACCCATAAATTGCTTTCAAAACTGTCAGTCTTGAGTGGTGTAGAGGTAGGCGGAATT
CCCGGTGTAGCGGTGGAATGCGTAGATATCGGGAGGAACACCAGTGGCGAAGGCG
GCCTACTGGGCACTAACTGACGCTGAGGCTCGAAAGCATGGGTAGCAAACAGGATT
AGATACCCTGGTAGTCCATGCCGTAAACGATGATTACTAGGTGTGGGAGGATTGAC
CCCTTCCGTGCCGCAGTTAACACAATAAGTAATCCACCTGGGGAGTACGACCGCAA
GGTTGAAACTCAAAGGAATTGACGGGGGCCCGCACAAGCAGTGGAGTATGTGGTTT
AATTCGAAGCAACGCGAAGAACCTTACCAGGTCTTGACATCGGATGCATACCTAAG
AGATTAGGGAAGTCCTTCGGGACATCCAGACAGGTGGTGCATGGTTGTCGTCAGCT
CGTGTCGTGAGATGTTGGGTTAAGTCCCGCAACGAGCGCAACCCTTATCGTTAGTT
ACTACGCAAGAGGACTCTAGCGAGACTGCCGTTGACAAAACGGAGGAAGGTGGGG
ATGACGTCAAATCATCATGCCCTTTATGACCTGGGCTACACACGTACTACAATGGCT
ATTAACAGAGAGAAGCGATACCGCGAGGTGGAGCAAACCTCACAAAAATAGTCTCA
GTTCGGATCGCAGGCTGCAACCCGCCTGCGTGAAGCCGGAATTGCTAGTAATCGC
GGATCAGCATGCCGCGGTGAATACGTTCCCGGGCCTTGTACACACCGCCCGTCAC
ACCATGAGAGCCGGGGGGACCCGAAGTCGGTAGTCTAACCGCAAGGAGGACGCCG
CCGAAGGTAAAACTGGTGATTGGGGTGAAGTCGTAACAAGGTAGCCGTATCGGAAG
GTGCGGCTGGATCACCTCCTTT
SEQ ID NO: 41 Paraprevotella xylaniphila
AATAAAGATTAATTGGTAAAGGATGGGGATGCGTCCCATTAGCTTGTTGGCGGGGT
AACGGCCCACCAAGGCGACGATGGGTAGGGGTTCTGAGAGGAAGGTCCCCCACAT
TGGAACTGAGACACGGTCCAAACTCCTACGGGAGGCAGCAGTGAGGAATATTGGTC
AATGGGCGCGAGCCTGAACCAGCCAAGTAGCGTGGAGGACGACGGCCCTACGGGT
TGTAAACTCCTTTTATAAGGGGATAAAGTTGGCCATGTATGGCCATTTGCAGGTACC
TTATGAATAAGCATCGGCTAATTCCGTGCCAGCAGCCGCGGTAATACGGAAGATGC
GAGCGTTATCCGGATTTATTGGGTTTAAAGGGAGCGTAGGCGGGCAGTCAAGTCAG
CGGTCAAATGGCGCGGCTCAACCGCGTTCCGCCGTTGAAACTGGCAGCCTTGAGT
ATGCACAGGGTACATGGAATTCGTGGTGTAGCGGTGAAATGCTTAGATATCACGAG
GAACTCCGATCGCGCAGGCATTGTACCGGGGCATTACTGACGCTGAGGCTCGAAG
GTGCGGGTATCAAACAGGATTAGATACCCTGGTAGTCCGCACAGTAAACGATGAAT
GCCCGCTGTCGGCGACATAGTGTCGGCGGCCAAGCGAAAGCGTTAAGCATTCCAC
CTGGGGAGTACGCCGGCAACGGTGAAACTCAAAGGAATTGACGGGGGCCCGCACA
AGCGGAGGAACATGTGGTTTAATTCGATGATACGCGAGGAACCTTACCCGGGCTTG
AATCGCAGGTGCATGGGCCGGAGACGGCCCTTTCCTTCGGGACTCCTGCGAAGGT
GCTGCATGGTTGTCGTCAGCTCGTGCCGTGAGGTGTCGGCTTAAGTGCCATAACGA
GCGCAACCCCCCTCCCCAGTTGCCACCGGGTAATGCCGGGCACTTTGGGGACACT
GCCACCGCAAGGTGCGAGGAAGGTGGGGATGACGTCAAATCAGCACGGCCCTTAC
GTCCGGGGCGACACACGTGTTACAATGGGGGGTACAGAGGGCCGCTGCCCGGTGA
CGGTTGGCCAATCCCTAAAACCCCTCTCAGTTCGGACTGGAGTCTGCAACCCGACT
CCACGAAGCTGGATTCGCTAGTAATCGCGCATCAGCCATGGCGCGGTGAATACGTT
CCCGGGCCTTGTACACACCGCCCGTCAAGCCATGAAAGCCGGGGGTGCCTGAAGT
CCGTGACCGCGAGGGTCGGCCTAGGGTAAAACCGGTGATTGGGGCTAAGTCGTAA
CAAGGTAGCCGTACCGGAAGGTGCGGCTGGAACACCTCCTTT
SEQ ID NO: 42 Parabacteroides johnsonii
CGAAGAGTTTGATCCTGGCTCAGGATGAACGCTAGCGACAGGCTTAACACATGCAA
GTCGAGGGGCATCATGGTAAGTAGCAATACTTATTGATGGCGACCGGCGCACGGGT
GAGTAACGCGTATGCAACTTACCTATCAGAGGGGGATAGCCCGGCGAAAGTCGGAT
TAATACTCCATAAAACAGGGGTTCCGCATGGGACTATTTGTTAAAGATTCATCGCTG
ATAGATAGGCATGCGTTCCATTAGGCAGTTGGCGGGGTAACGGCCCACCAAACCGA
CGATGGATAGGGGTTCTGAGAGGAAGGTCCCCCACATTGGTACTGAGACACGGAC
CAAACTCCTACGGGAGGCAGCAGTGAGGAATATTGGTCAATGGCCGAGAGGCTGA
ACCAGCCAAGTCGCGTGAAGGATGAAGGATCTATGGTTTGTAAACTTCTTTTATAGG
GGAATAAAGTGTGGGACGTGTTCCATTTTGTATGTACCCTATGAATAAGCATCGGCT
AACTCCGTGCCAGCAGCCGCGGTAATACGGAGGATGCGAGCGTTATCCGGATTTAT
TGGGTTTAAAGGGTGCGTAGGTGGTAATTTAAGTCAGCGGTGAAAGTTTGTGGCTC
AACCATAAAATTGCCGTTGAAACTGGGTTACTTGAGTGTGTTTGAGGTAGGCGGAAT
GCGTGGTGTAGCGGTGAAATGCATAGATATCACGCAGAACTCCAATTGCGAAGGCA
GCTTACTAAACCATAACTGACACTGAAGCACGAAAGCGTGGGTATCAAACAGGATTA
GATACCCTGGTAGTCCACGCAGTAAACGATGATTACTAGGAGTTTGCGATACACAG
TAAGCTCTACAGCGAAAGCGTTAAGTAATCCACCTGGGGAGTACGCCGGCAACGGT
GAAACTCAAAGGAATTGACGGGGGCCCGCACAAGCGGAGGAACATGTGGTTTAATT
CGATGATACGCGAGGAACCTTACCCGGGTTTGAACGTAGTCAGACCGACCTTGAAA
GAGGTTTTCTAGCAATAGCTGATTACGAGGTGCTGCATGGTTGTCGTCAGCTCGTG
CCGTGAGGTGTCGGCTTAAGTGCCATAACGAGCGCAACCCTTATCACTAGTTACTA
ACAGGTTAAGCTGAGGACTCTGGTGAGACTGCCAGCGTAAGCTGTGAGGAAGGTG
GGGATGACGTCAAATCAGCACGGCCCTTACATCCGGGGCGACACACGTGTTACAAT
GGCATGGACAAAGGGCAGCTACCTGGCGACAGGATGCTAATCTCTAAACCATGTCT
CAGTTCGGATCGGAGTCTGCAACTCGACTCCGTGAAGCTGGATTCGCTAGTAATCG
CGCATCAGCCATGGCGCGGTGAATACGTTCCCGGGCCTTGTACACACCGCCCGTC
AAGCCATGGGAGCCGGGGGTACCTGAAGTCCGTAACCGCAAGGATCGGCCTAGGG
TAAAACTGGTGACTGGGGCTAAGTCGTAACAAGGTAGCCGTACCGGAAGGTGCGG
CTGGAACACCTCCTT
SEQ ID NO: 43 Alistipes senegalensis
ATGGAGAGTTTGATCCTGGCTCAGGATGAACGCTAGCGGCAGGCCTAACACATGCA
AGTCGAGGGGCAGCGGGATTGAAGCTTGCTTCAGTTGCCGGCGACCGGCGCACGG
GTGCGTAACGCGTATGCAACCTACCCATAACAGGGGGATAACACTGAGAAATCGGT
ACTAATATCCCATAACATCAAGAGGGGCATCCCTTTTGGTTGAAAACTCCGGTGGTT
ATGGATGGGCATGCGTTGTATTAGCTAGTTGGTGAGGTAACGGCTCACCAAGGCGA
CGATACATAGGGGGACTGAGAGGTTAACCCCCCACATTGGTACTGAGACACGGACC
AAACTCCTACGGGAGGCAGCAGTGAGGAATATTGGTCAATGGACGCAAGTCTGAAC
CAGCCATGCCGCGTGCAGGATGACGGCTCTATGAGTTGTAAACTGCTTTTGTACGA
GGGTAAACCCGGATACGTGTATCCGGCTGAAAGTATCGTACGAATAAGGATCGGCT
AACTCCGTGCCAGCAGCCGCGGTAATACGGAGGATTCAAGCGTTATCCGGATTTAT
TGGGTTTAAAGGGTGCGTAGGCGGTTTGATAAGTTAGAGGTGAAATACCGGTGCTT
AACACCGGAACTGCCTCTAATACTGTTGAGCTAGAGAGTAGTTGCGGTAGGCGGAA
TGTATGGTGTAGCGGTGAAATGCTTAGAGATCATACAGAACACCGATTGCGAAGGC
AGCTTACCAAACTATATCTGACGTTGAGGCACGAAAGCGTGGGGAGCAAACAGGAT
TAGATACCCTGGTAGTCCACGCAGTAAACGATGATAACTCGCTGTCGGCGATACAC
AGTCGGTGGCTAAGCGAAAGCGATAAGTTATCCACCTGGGGAGTACGTTCGCAAGA
ATGAAACTCAAAGGAATTGACGGGGGCCCGCACAAGCGGAGGAACATGTGGTTTAA
TTCGATGATACGCGAGGAACCTTACCCGGGCTTGAAAGTTACTGACGATTCTGGAA
ACAGGATTTCCCTTCGGGGCAGGAAACTAGGTGCTGCATGGTTGTCGTCAGCTCGT
GCCGTGAGGTGTCGGGTTAAGTCCCATAACGAGCGCAACCCCTACCGTTAGTTGCC
ATCAGGTCAAGCTGGGCACTCTGGCGGGACTGCCGGTGTAAGCCGAGAGGAAGGT
GGGGATGACGTCAAATCAGCACGGCCCTTACGTCCGGGGCTACACACGTGTTACAA
TGGTAGGTACAGAGGGCAGCTACCCAGTGATGGGATGCGAATCTCGAAAGCCTATC
TCAGTTCGGATTGGAGGCTGAAACCCGCCTCCATGAAGTTGGATTCGCTAGTAATC
GCGCATCAGCCATGGCGCGGTGAATACGTTCCCGGGCCTTGTACACACCGCCCGT
CAAGCCATGGAAGCTGGGGGTGCCTGAAGTTCGTGACCGCAAGGAGCGACCTAGG
GCAAAACCGGTGACTGGGGCTAAGTCGTAACAAGGTAGCCGTACCGGAAGGTGCG
GCTGGAACACCTCCTTT
SEQ ID NO: 44 Parabacteroides gordonii
CGAAGAGTTTGATCCTGGCTCAGGATGAACGCTAGCGACAGGCTTAACACATGCAA
GTCGAGGGGCAGCAGGAAGTAGCAATACTTTGCTGGCGACCGGCGCACGGGTGAG
TAACGCGTATGCAACCTACCTATCAGAGGGGGATAACCCGGCGAAAGTCGGACTAA
TACCGCATAAAACAGGGGTCCCGCATGGGAATATTTGTTAAAGATTTATTGCTGATA
GATGGGCATGCGTTCCATTAGATAGTTGGTGAGGTAACGGCTCACCAAGTCTTCGA
TGGATAGGGGTTCTGAGAGGAAGGTCCCCCACACTGGTACTGAGACACGGACCAG
ACTCCTACGGGAGGCAGCAGTGAGGAATATTGGTCAATGGGCGAGAGCCTGAACC
AGCCAAGTCGCGTGAAGGATGAAGGATCTATGGTTCGTAAACTTCTTTTATAGGGG
AATAAAGTGCAGGACGTGTCCTGTTTTGTATGTACCCTATGAATAAGGATCGGCTAA
CTCCGTGCCAGCAGCCGCGGTAATACGGAGGATCCGAGCGTTATCCGGATTTATTG
GGTTTAAAGGGTGCGTAGGTGGCTTTTTAAGTCAGCGGTGAAAGTTTGTGGCTCAA
CCATAAAATTGCCGTTGAAACTGGAGGGCTTGAGTATATTTGAGGTAGGCGGAATG
CGTGGTGTAGCGGTGAAATGCATAGATATCACGCAGAACTCCAATTGCGAAGGCAG
CTTACTAAACTATAACTGACACTGAAGCACGAAAGCGTGGGGATCAAACAGGATTA
GATACCCTGGTAGTCCACGCAGTAAACGATGATTACTAGGAGTTTGCGATACACAG
TAAGCTCTACAGCGAAAGCGTTAAGTAATCCACCTGGGGAGTACGCCGGCAACGGT
GAAACTCAAAGGAATTGACGGGGGCCCGCACAAGCGGAGGAACATGTGGTTTAATT
CGATGATACGCGAGGAACCTTACCCGGGTTTGAACGTAAGTTGACCGGAGTGGAAA
CACTCTTTCTAGCAATAGCAATTTACGAGGTGCTGCATGGTTGTCGTCAGCTCGTGC
CGTGAGGTGTCGGCTTAAGTGCCATAACGAGCGCAACCCTTATCTTTAGTTACTAAC
AGGTCGAGCTGAGGACTCTAAAGAGACTGCCAGCGTAAGCTGTGAGGAAGGTGGG
GATGACGTCAAATCAGCACGGCCCTTACATCCGGGGCGACACACGTGTTACAATGG
TGGGGACAAAGGGCAGCTACCTGGCGACAGGATGCTAATCTCCAAACCCCATCTCA
GTTCGGATCGAAGTCTGCAACCCGACTTCGTGAAGCTGGATTCGCTAGTAATCGCG
CATCAGCCATGGCGCGGTGAATACGTTCCCGGGCCTTGTACACACCGCCCGTCAA
GCCATGGGAGTTGGGGGTACCTAAAGTCCGTAACCGCAAGGATCGGCCTAGGGTA
AAACCGATGACTGGGGCTAAGTCGTAACAAGGTAGCCGTACCGGAAGGTGCGGCT
GGAACACCTCCTTT
SEQ ID NO: 45 Eubacterium limosum
TATTGAGAGTTTGATCCTGGCTCAGGACGAACGCTGGCGGTATGCTTAACACATGC
AAGTCGAACGAGAAGGTTTTGATGGATCCTTCGGGTGATATCAGAACTGGAAAGTG
GCGAACGGGTGAGTAACGCGTGGGTAACCTGCCCTATGGAAAGGAATAGCCTCGG
GAAACTGGGAGTAAAGCCTTATATTATGGTTTTGTCGCATGGCAAGATCATGAAAAC
TCCGGTGCCATAGGATGGACCCGCGTCCCATTAGCTAGTTGGTGAGATAACAGCCC
ACCAAGGCGACGATGGGTAACCGGTCTGAGAGGGCGAACGGTCACACTGGAACTG
AGACACGGTCCAGACTCCTACGGGAGGCAGCAGTGGGGAATATTGCGCAATGGGG
GCAACCCTGACGCAGCAATACCGCGTGAGTGAAGAAGGTTTTCGGATCGTAAAGCT
CTGTTATTGGGGAAGAAGAATGACGGTACCCAATGAGGAAGTCCCGGCTAACTACG
TGCCAGCAGCCGCGGTAATACGTAGGGGACAAGCGTTGTCCGGAATGACTGGGCG
TAAAGGGCGCGTAGGCGGTCTATTAAGTCTGATGTGAAAGGTACCGGCTCAACCGG
TGAAGTGCATTGGAAACTGGTAGACTTGAGTATTGGAGAGGCAAGTGGAATTCCTA
GTGTAGCGGTGAAATGCGTAGATATTAGGAGGAACACCAGTGGCGAAGGCGGCTT
GCTGGACAAATACTGACGCTGAGGTGCGAAAGCGTGGGGAGCGAACAGGATTAGA
TACCCTGGTAGTCCACGCCGTAAACGATGAATGCTAGGTGTTGGGGAAACTCAGTG
CCGCAGTTAACACAATAAGCATTCCGCCTGGGGAGTACGACCGCAAGGTTGAAACT
CAAAGGAATTGACGGGGACCCGCACAAGCAGCGGAGCATGTGGTTTAATTCGAAGC
AACGCGAAGAACCTTACCAGGTCTTGACATCCTCTGACGAGCCTAGAGATAGGAAG
TTTCCTTCGGGAACAGAGAGACAGGTGGTGCATGGTTGTCGTCAGCTCGTGTCGTG
AGATGTTGGGTTAAGTCCCGCAACGAGCGCAACCCCTGCCTTTAGTTGCCAGCATT
AAGTTGGGCACTCTAGAGGGACTGCCGTAGACAATACGGAGGAAGGTGGGGACGA
CGTCAAATCATCATGCCCCTTATGACCTGGGCTACACACGTGCTACAATGGTCTGAA
CAGAGGGCCGCGAAGCCGCGAGGTGAAGCAAATCCCTTAAAACAGATCCCAGTTC
GGATTGCAGGCTGCAACTCGCCTGCATGAAGTTGGAGTTGCTAGTAATCGCGGATC
AGAATGCCGCGGTGAATGCGTTCCCGGGTCTTGTACACACCGCCCGTCACACCAC
GAGAGTTGGCAACACCCGAAGCCTGTGAGAGAACCGTAAGGACTCAGCAGTCGAA
GGTGGGGCTAGTAATTGGGGTGAAGTCGTAACAAGGTAGCCGTATCGGAAGGTGC
GGCTGGATCACCTCCTTT
SEQ ID NO: 46 Parabacteroides distasonis
CGAAGAGTTTGATCCTGGCTCAGGATGAACGCTAGCGACAGGCTTAACACATGCAA
GTCGAGGGGCAGCACAGGTAGCAATACCGGGTGGCGACCGGCGCACGGGTGAGT
AACGCGTATGCAACTTGCCTATCAGAGGGGGATAACCCGGCGAAAGTCGGACTAAT
ACCGCATGAAGCAGGGGCCCCGCATGGGGATATTTGCTAAAGATTCATCGCTGATA
GATAGGCATGCGTTCCATTAGGCAGTTGGCGGGGTAACGGCCCACCAAACCGACG
ATGGATAGGGGTTCTGAGAGGAAGGTCCCCCACATTGGTACTGAGACACGGACCAA
ACTCCTACGGGAGGCAGCAGTGAGGAATATTGGTCAATGGCCGAGAGGCTGAACC
AGCCAAGTCGCGTGAGGGATGAAGGTTCTATGGATCGTAAACCTCTTTTATAAGGG
AATAAAGTGCGGGACGTGTCCCGTTTTGTATGTACCTTATGAATAAGGATCGGCTAA
CTCCGTGCCAGCAGCCGCGGTAATACGGAGGATCCGAGCGTTATCCGGATTTATTG
GGTTTAAAGGGTGCGTAGGCGGCCTTTTAAGTCAGCGGTGAAAGTCTGTGGCTCAA
CCATAGAATTGCCGTTGAAACTGGGGGGCTTGAGTATGTTTGAGGCAGGCGGAATG
CGTGGTGTAGCGGTGAAATGCATAGATATCACGCAGAACCCCGATTGCGAAGGCAG
CCTGCCAAGCCATTACTGACGCTGATGCACGAAAGCGTGGGGATCAAACAGGATTA
GATACCCTGGTAGTCCACGCAGTAAACGATGATCACTAGCTGTTTGCGATACACTGT
AAGCGGCACAGCGAAAGCGTTAAGTGATCCACCTGGGGAGTACGCCGGCAACGGT
GAAACTCAAAGGAATTGACGGGGGCCCGCACAAGCGGAGGAACATGTGGTTTAATT
CGATGATACGCGAGGAACCTTACCCGGGTTTGAACGCATTCGGACCGAGGTGGAAA
CACCTTTTCTAGCAATAGCCGTTTGCGAGGTGCTGCATGGTTGTCGTCAGCTCGTG
CCGTGAGGTGTCGGCTTAAGTGCCATAACGAGCGCAACCCTTGCCACTAGTTACTA
ACAGGTAAAGCTGAGGACTCTGGTGGGACTGCCAGCGTAAGCTGCGAGGAAGGCG
GGGATGACGTCAAATCAGCACGGCCCTTACATCCGGGGCGACACACGTGTTACAAT
GGCGTGGACAAAGGGAAGCCACCTGGCGACAGGGAGCGAATCCCCAAACCACGTC
TCAGTTCGGATCGGAGTCTGCAACCCGACTCCGTGAAGCTGGATTCGCTAGTAATC
GCGCATCAGCCATGGCGCGGTGAATACGTTCCCGGGCCTTGTACACACCGCCCGT
CAAGCCATGGGAGCCGGGGGTACCTGAAGTCCGTAACCGCGAGGATCGGCCTAGG
GTAAAACTGGTGACTGGGGCTAAGTCGTAACAAGGTAGCCGTACCGGAAGGTGCG
GCTGGAACACCTCCTTT

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Rationally Designed Bacterial Compositions for Use in Cancer Therapy

Efficacy of immune checkpoint inhibitor therapy (ICT) therapy is associated with the composition of the patient's gut microbiome, however the mechanism remains to be elucidated. Recent data has demonstrated that a consortium of 11 bacterial strains, referred to as VE800, induced IFNγ production in intestinal CD8+ T cells and enhanced anti-PD-1 cancer activity in tumor models (Tanoue et al. Nature (2019) 565(7741): 600-605).

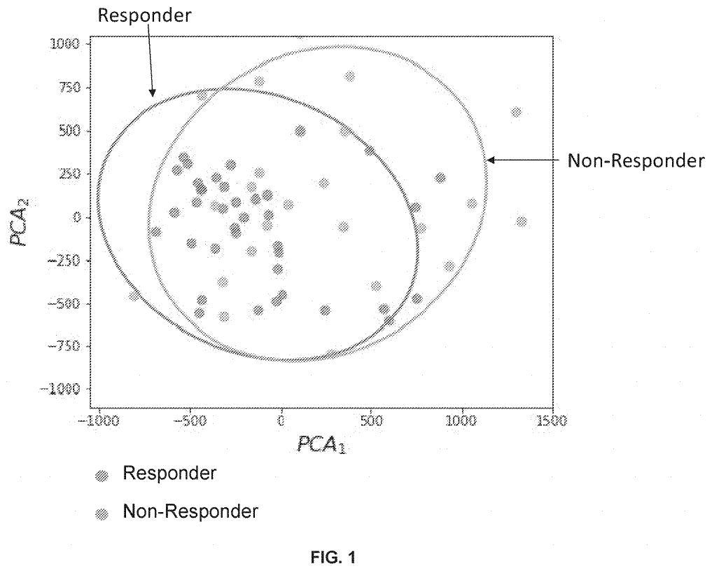

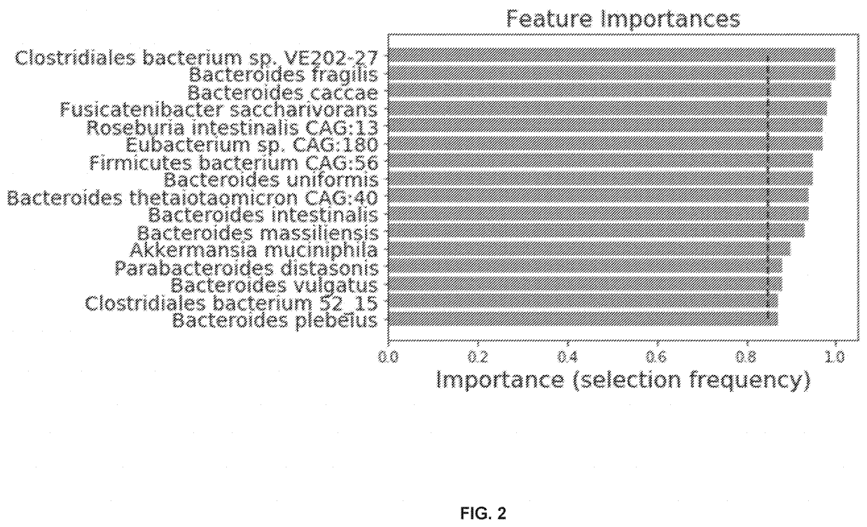

To understand the response to immune checkpoint inhibitor therapy (ICT), the composition of the gut microbiome of melanoma patients that responded to checkpoint therapy (Responders) versus those that did not respond (Non-Responders) was examined by whole genome shotgun sequencing (WGS). The beta-diversity (Bray-Curtis) was found to be significantly different between the Responder cohort versus the Non-Responder cohort (FIG. 1). Sparse regression analysis demonstrated bacterial taxa that are important in alterations to the gut microbiome associated with checkpoint therapy (FIG. 2).

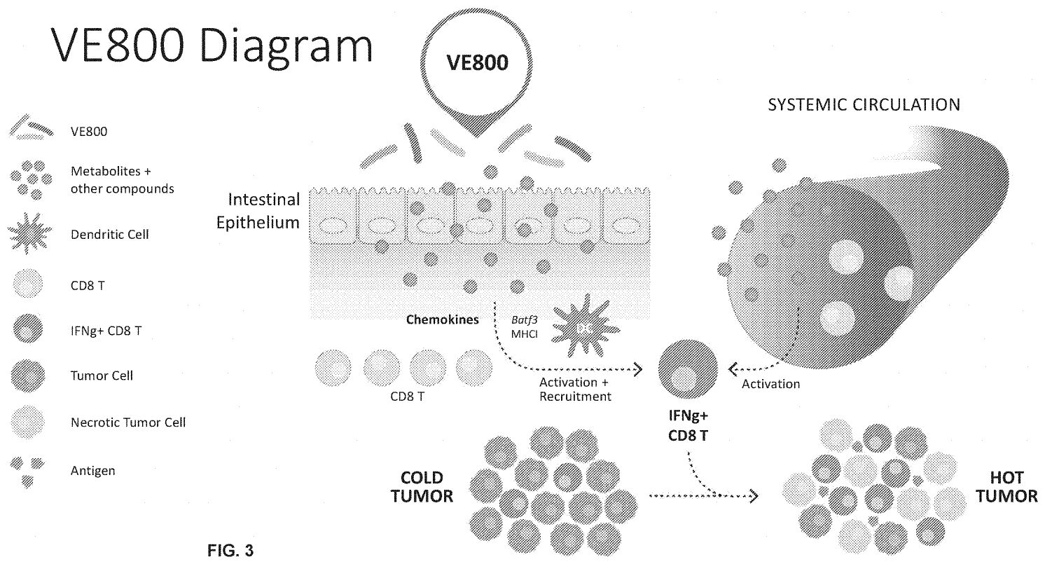

As shown in FIG. 3, without being limited to any particular theory, it is considered that colonization by specific commensal bacterial strains induce immunostimulatory responses in germ-free mice, including IFNγ CD8+ T cells, that provide anti-tumor activity.

### Induction of IFNγCD8+ T Cells

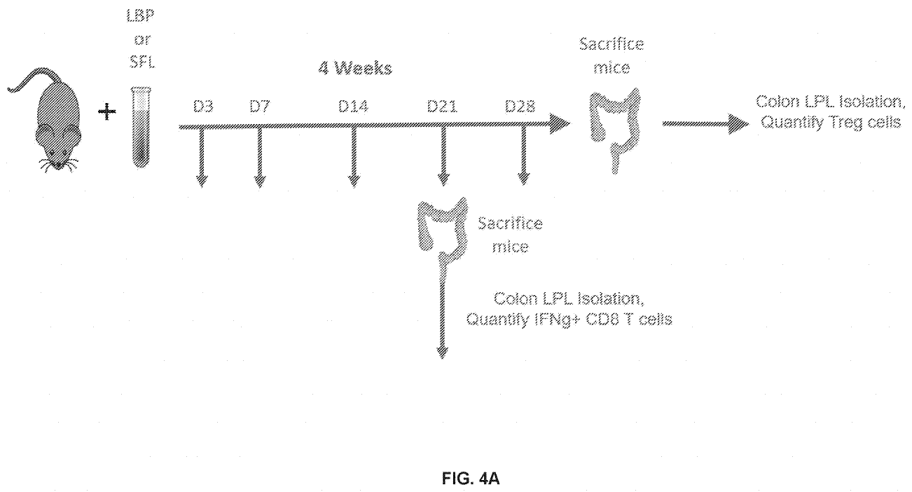

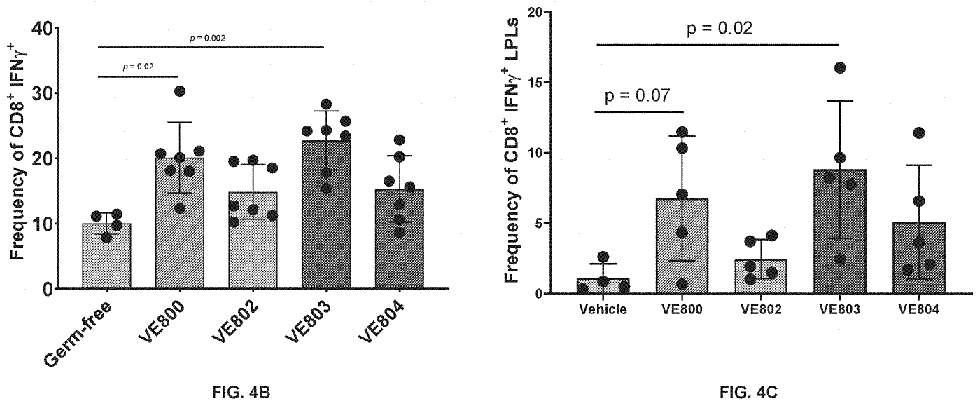

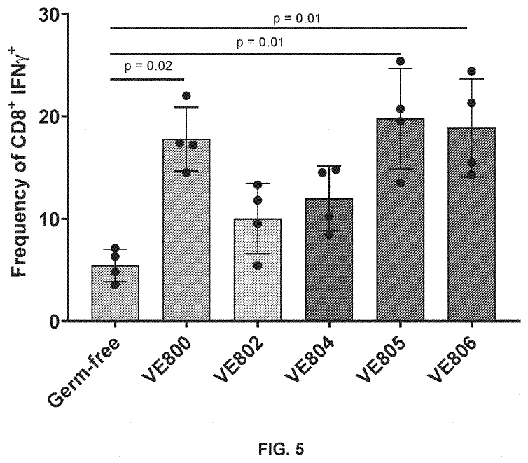

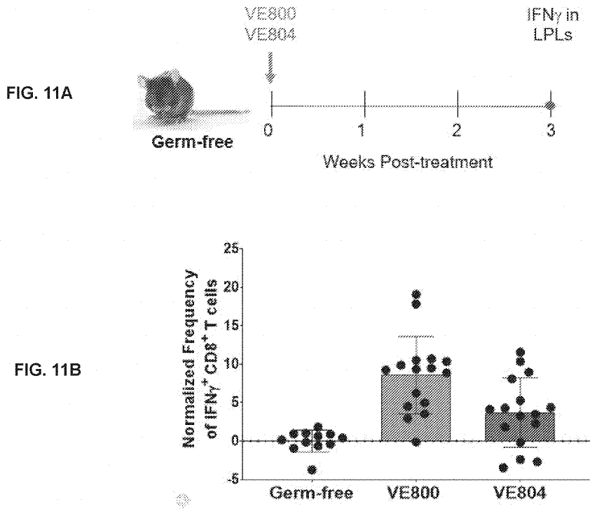

Derivative compositions of VE800 were developed containing subset of bacterial strains to determine the consortia involved in stimulating immune phenotypes in germ-free mice (FIGS. 4A, 11A). See, Tables 2-6. Bacterial compositions VE802 and VE804 bacterial consortia, which did not contain Fusobacterium ulcerans, were found to induce reduced levels of IFNγ CD8⁺ T cells as compared to bacterial composition VE800 or VE803 (FIGS. 4B, 4C, and 11B), whereas bacterial compositions VE805 and VE806 retained IFNγ-inducing activity (FIGS. 5A and 5B). These results suggest that Fusobacterium ulcerans contributes to stimulation of IFNγ CD8+ T cells.

### B16 Melanoma Model

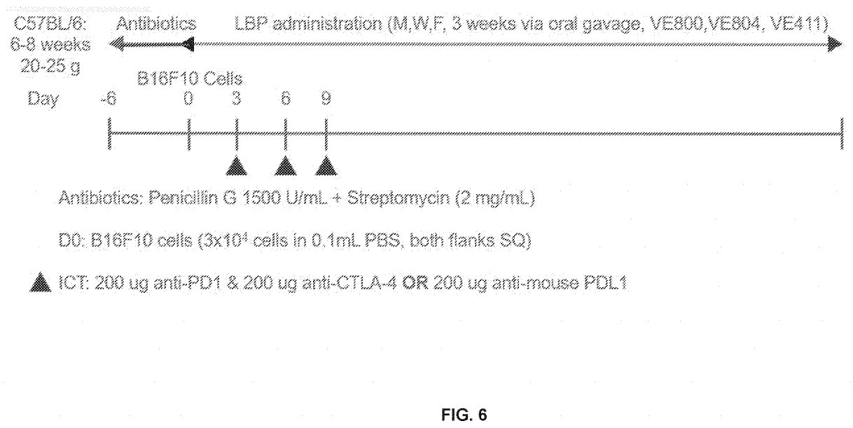

C57BL/6 germ-free mice were administered antibiotics (penicillin+streptomycin) for 6 days, followed by subcutaneous injection with 3×10⁴ B16F10 melanoma cells and administration live bacterial products (“LBP”): VE800 (“11-mix”) or derivative compositions thereof (e.g., VE804), or control bacterial composition VE411 (Clostridium saccharogumia, Clostridium ramosum, Blautia producta, and Eubacterium contortum). On days 3, 6, and 9 after injection with melanoma cells, the mice were treated with immune checkpoint therapy (ICT) (FIG. 6).

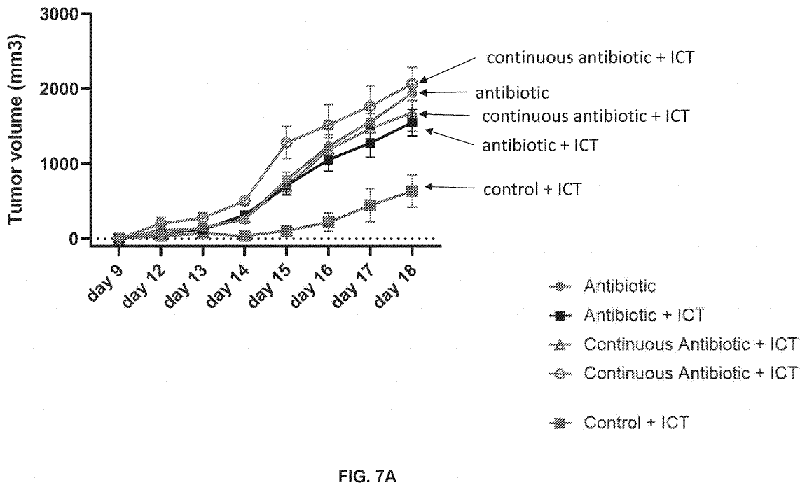

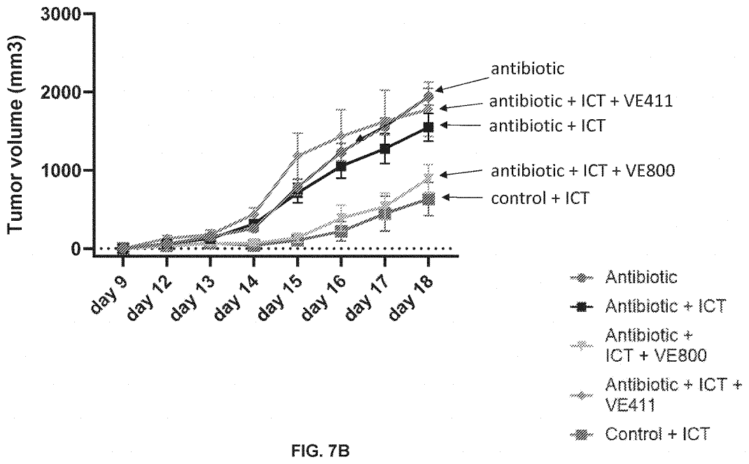

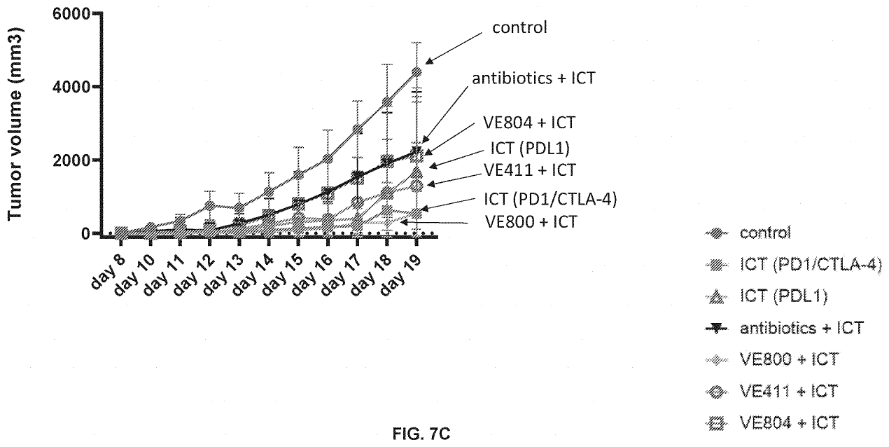

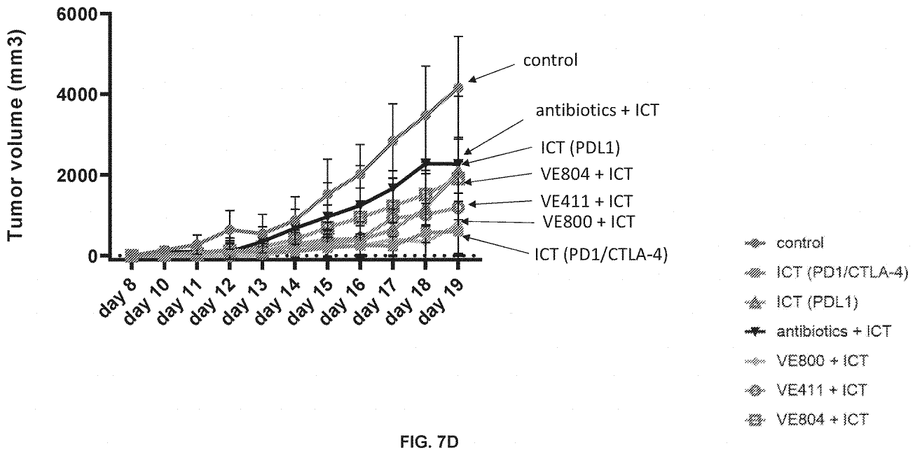

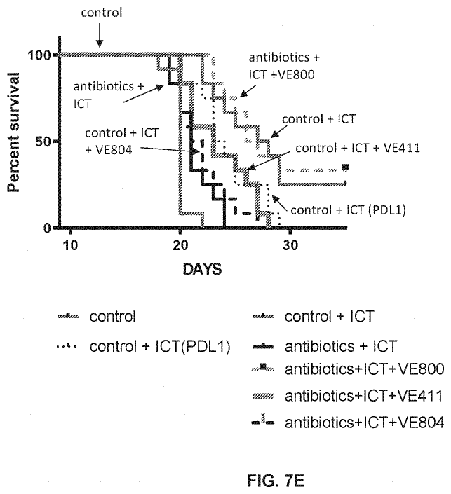

As shown in FIG. 7A, treatment with antibiotics prior to injection of melanoma cells or continuously throughout the experiment abolished the anti-tumor activity of ICT. Administration of specific bacterial compositions, such as VE800 but not VE804 or VE411, restored the efficacy ICT, comparable to levels of ICT alone (FIGS. 7B-7D). Survival of the mice injected with melanoma cells was also monitored. Administration of bacterial composition VE800 was found to promote survival to comparable levels as ICT alone (FIG. 7E).

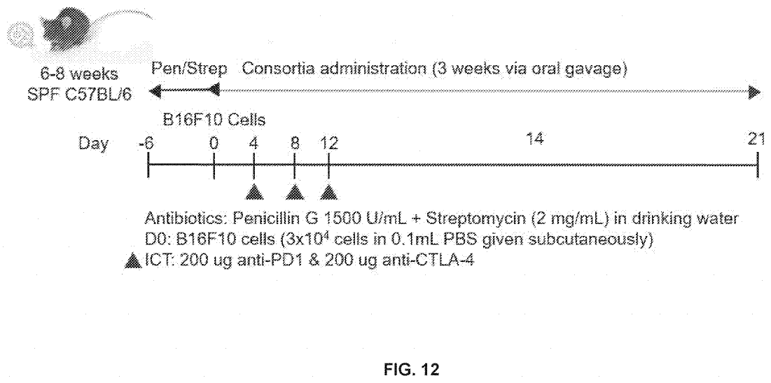

C57BL/6 SPF mice were administered antibiotics (penicillin+streptomycin) for 6 days, followed by subcutaneous injection with 3×10⁴ B16F10 melanoma cells and administration of live bacterial compositions. On days 4, 8, and 12 after injection with melanoma cells, the mice were treated with immune checkpoint therapy (ICT) (FIG. 12).

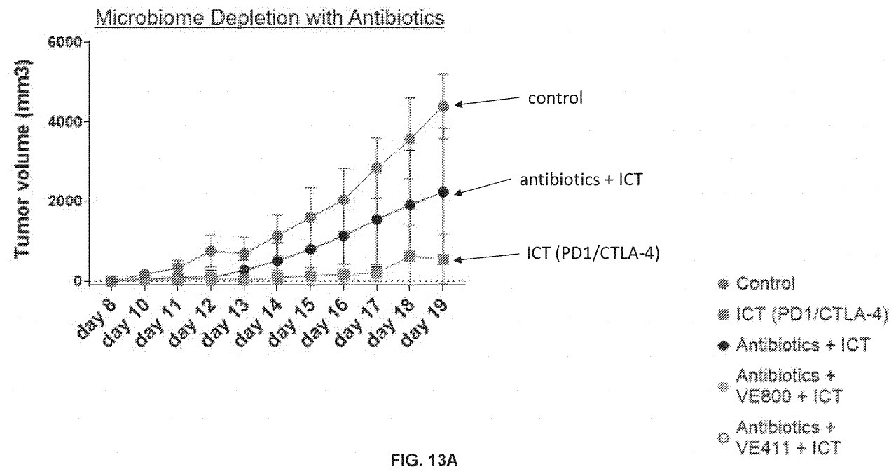

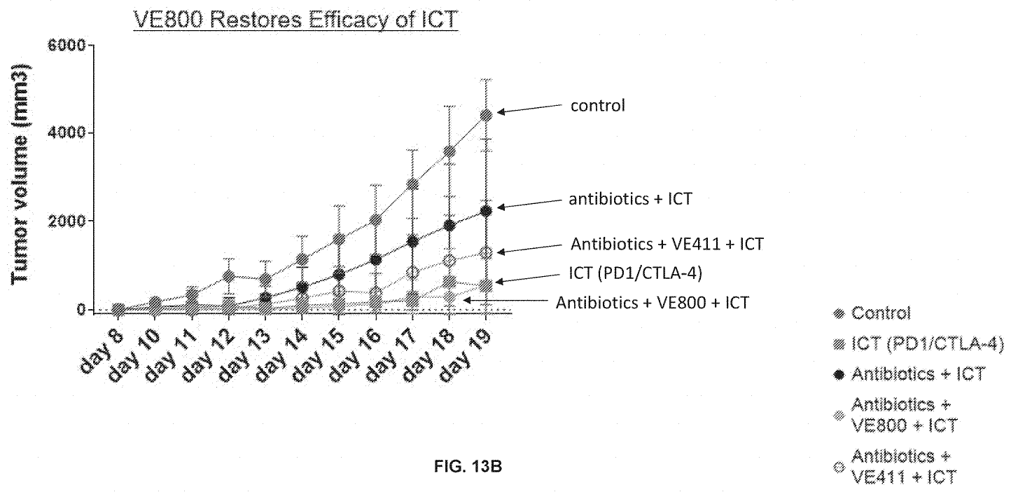

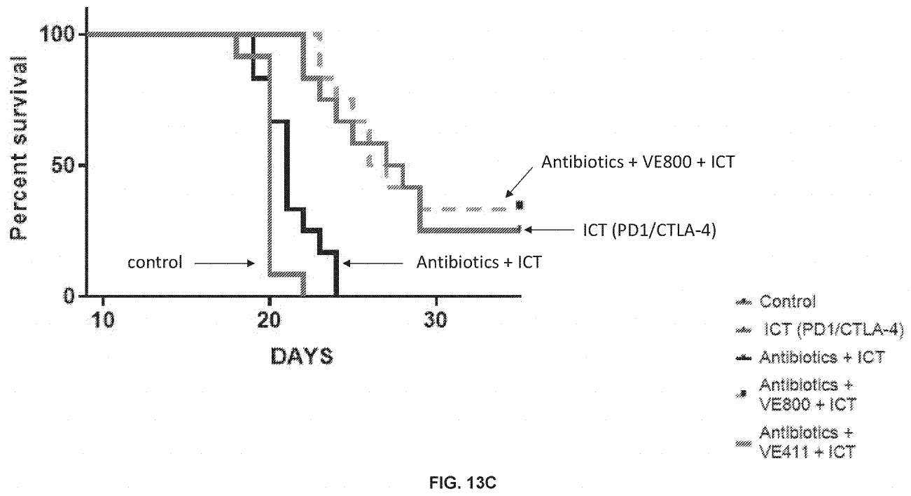

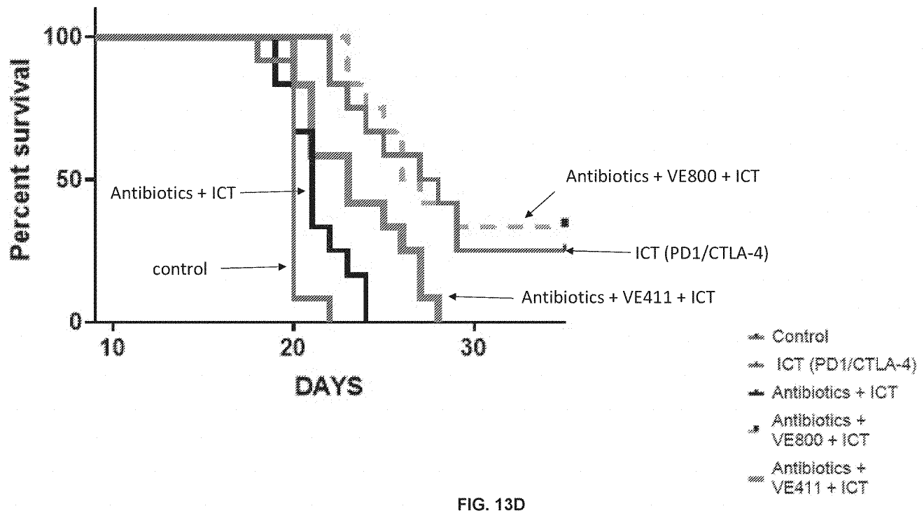

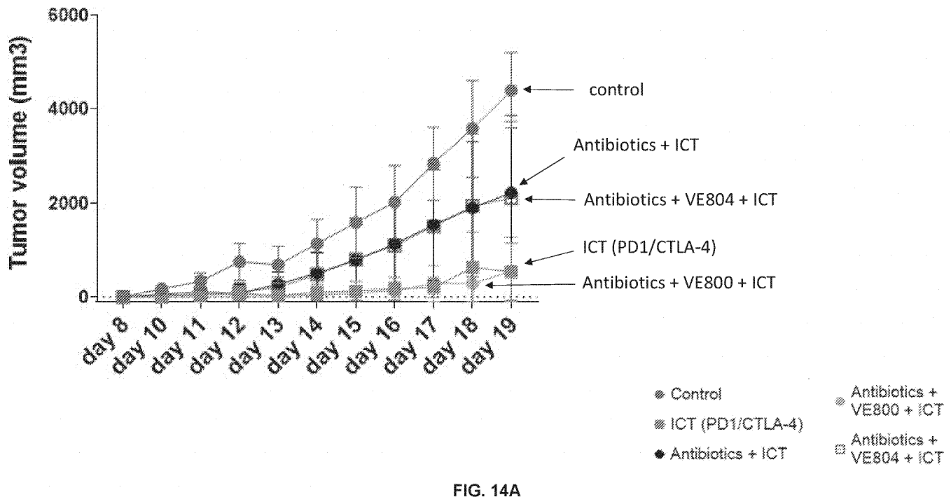

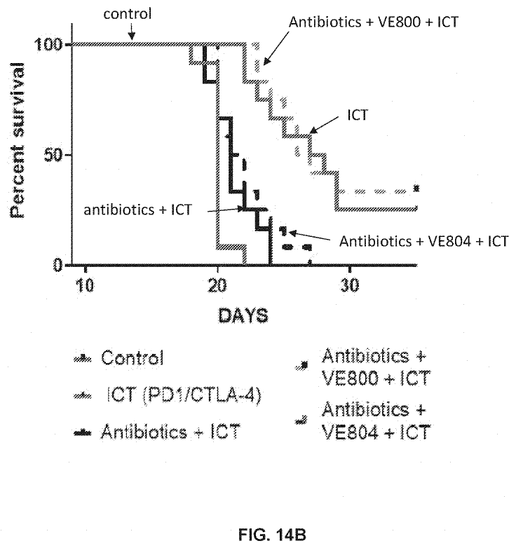

As shown in FIG. 13A, treatment with antibiotics prior to injection of melanoma cells abolished the anti-tumor activity of ICT. Administration of bacterial composition VE800, but not VE411, restored the efficacy ICT, comparable to levels of ICT alone (FIG. 13B). Survival of the mice injected with melanoma cells was also monitored. Administration of bacterial composition VE800 was found to promote survival to comparable levels as ICT alone (FIGS. 13C and 13D). Additionally, bacterial compositions that were found to induce reduced levels of IFNγ (i.e., VE804), were found to have reduced anti-tumor activity, including reduced survival (FIGS. 14A and 14B).

### Induction of Regulatory T Cells

Immune checkpoint inhibition is effective in killing cancer cells but may cause colitis as a result of increased immune activity. Local induction of regulatory T cells (Tregs) may be beneficial in prevent of checkpoint therapy-induced colitis. The bacterial compositions were also assessed for regulatory T cell (Treg) stimulatory activity (FIG. 11A). Bacterial composition VE800 contains two strains that are putative inducers of Tregs: Ruminococcaceae sp. and Eubacterium.

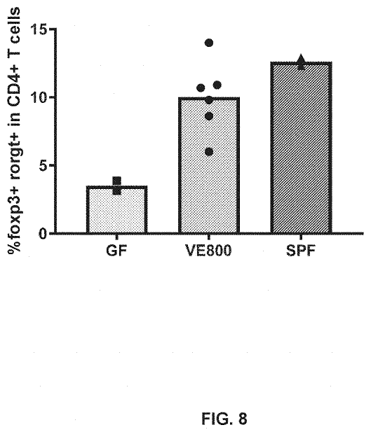

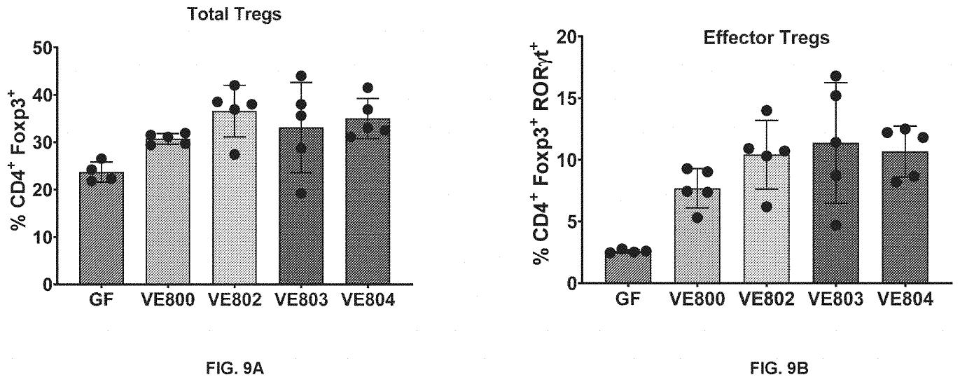

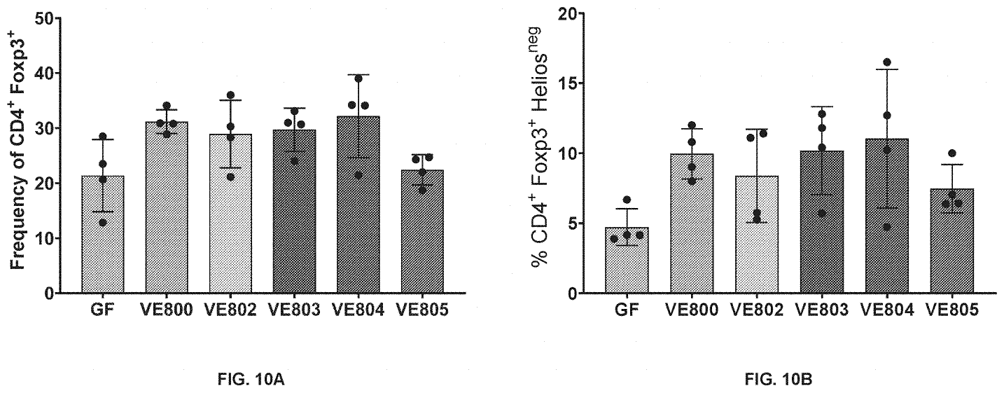

Administration of bacterial composition VE800 resulted in induction of increased levels of Treg cells (FIG. 8). VE802 and VE804, which induced reduced levels of IFNγ CD8⁺ T cells, were found to retain Treg stimulatory activity (FIGS. 9A and 9B), whereas bacterial composition VE805 induced reduced levels of Treg cells (FIGS. 10A and 10B). These results suggest that the putative Treg inducer strains Eubacterium and/or Ruminococcaceae indeed contribute to induction of Treg cells by the full VE800 consortium.

TABLE 2
Bacterial strains of composition VE802
VE802

Parabacteroides johnsonii
Paraprevotella xylaniphila
Parabacteroides distasonis
Alistipes sp.
Eubacterium limosum
Bacteroides dorei
Parabacteroides gordonii
Subdolinogranulum sp.
Bacteroides uniformis
Phascolarctobacterium faecium

TABLE 3
Bacterial strains of composition VE803
VE803

Fusobacterium ulcerans
Parabacteroides johnsonii
Paraprevotella xylaniphila
Parabacteroides distasonis
Alistipes sp.
Eubacterium limosum
Bacteroides dorei
Parabacteroides gordonii
Bacteroides uniformis
Phascolarctobacterium faecium

TABLE 4
Bacterial strains of composition VE804
VE804

Parabacteroides johnsonii
Paraprevotella xylaniphila
Parabacteroides distasonis
Alistipes sp.
Eubacterium limosum
Bacteroides dorei
Parabacteroides gordonii
Bacteroides uniformis
Phascolarctobacterium faecium

TABLE 5
Bacterial strains of composition VE805
VE805

Fusobacterium ulcerans
Parabacteroides johnsonii
Paraprevotella xylaniphila
Parabacteroides distasonis
Alistipes sp.
Bacteroides dorei
Parabacteroides gordonii
Bacteroides uniformis
Phascolarctobacterium faecium

TABLE 6
Bacterial strains of composition VE806
VE806

Fusobacterium ulcerans
Parabacteroides johnsonii
Paraprevotella xylaniphila
Parabacteroides distasonis
Alistipes sp.
Bacteroides dorei
Parabacteroides gordonii
Subdolinogranulum sp.
Bacteroides uniformis
Phascolarctobacterium faecium

### Methods

### Bacterial Composition Preparation

The bacterial strains of composition VE800 were grown anaerobically as single colonies and inoculated into specific media that was optimal for each strain. Once all the strains reached stationary phase, an equal volume was taken from each strain and was pooled together to create the VE800 LBP. The pooled LBP was washed, and the pellet was resuspended and frozen in PBS+15% glycerol at −80° C. until the time of inoculation. Estimation of colony forming units (CFU)/dose was determined after freeze-thaw to quantitate the inoculum received by the mice. Derivative compositions containing subsets of the strains of VE800 were prepared as shown in Tables 2-6. A control 4 strain mix of anti-inflammatory Clostridia (VE411) was prepared with similar culturing conditions and preparation for gavage.

### Mouse Melanoma Model

Germ-free or specific pathogen free (SPF) C57BL/6 female mice between 6-8 wks old and 20-25 g in weight were purchased from Jackson Laboratories (Bar Harbor, Me.), and groups of 4 mice were placed in sterile bonneted cages with autoclaved water/mouse chow/bedding. The right flank or both flanks of the mice were shaved, and the animals were ear punched. Cohorts were given filtered (0.22 μm PVDF membranes) antibiotic water (penicillin G 0.95 g/L from GIBCO and streptomycin sulfate 2 g/L from VWR) for six days.

B16F10 mouse melanoma cells from ATCC (Rockville, Md.) were grown in DMEM with 4.5 g/L glucose with 10% heat inactivated fetal calf serum and penicillin/streptomycin and confluent cells were treated with 0.25 trypsin-EDTA, mixed with serum containing media, counted and 10⁵ passaged cells in 0.1 mL were injected subcutaneously in the right flank on each mouse. All media reagents were obtained from GIBCO. Starting on the day of tumor inoculation, some cohorts were given by gavage 0.2 mL of the bacterial compositions three times weekly for two weeks. At specific time points after tumor cell inoculation, mice were given intraperitoneal injections with 0.2 mL PBS containing 200 μg each of anti-mouse PD1 antibody (RMP1-14, Bio X Cell, West Lebanon, N.H.) and anti-CTLA4 antibody (9D9, Bio X Cell). Beginning day 8 post-tumor cell inoculation, tumor volumes will be measured with calipers employing the equation: V=(π/6)*length*width*height. Animals bearing tumors >2,500 cm³ or moribund were painlessly euthanized with CO₂ asphyxiation. In selected experiments, mice were euthanized, and tumor tissue isolated for studies described below. Fecal samples were collected 14 days post-tumor inoculation, snap frozen on dry ice and stored at −80° C. until assayed as described below. Tumor volumes versus time and Kaplan-Meier survival curves were generated using GraphPad Prism 7.0b software (San Diego, Calif.).

### Quantitation of Taxa in Mouse Stools

Fecal gDNA was using the QIAamp PowerFecal DNA kit with an initial step of bead-beating with phenol-chloroform. Isolated DNA was used in qPCR reactions or analyzed by whole genome shotgun sequencing on the Illumina platform at DNA Genotek, Inc., followed quality control and processing using the One Codex platform.

qPCR reaction mixes were prepared with 2.5-5 ng gDNA, 0.3 μM primers (Table 7) and Taqman Fast Universal PCR master mix. qPCR reactions were performed in Axygen 96-well plates on a Thermo Fisher QuantStudio 5 real time system. Control gDNAs were prepared from purified preparations of the 11 different VE800 strains. The mean cycle threshold for duplicate samples was calculated, and interpolated DNA concentration was ascertained. Interpolated DNA concentration was divided by the concentration of input DNA and multiplied by 100 to calculate the percent DNA of the target out of the total DNA.

TABLE 7
qPCR primers

| Strain | | | SEQ ID |
|---|---|---|---|
| designation | Species* | Forward and reverse primers | NO: |
| 81A6 | Fusobacterium ulcerans | 5’-TGCACTGTTGGATTTTCTAAAAAGG-3’, | 12 |
| | | 5’-ACTTTGGGCATGCTAAACCA-3’ | 13 |
| 81B11 | Bacteroides dorei | 5’-CGCCGGATGCATATACAAGA-3’, | 14 |
| | | 5’-TCTCGCCAATGATGTCCAAA-3’ | 15 |
| 81C1 | Eubacterium limosum | 5’-CCGCACAAGAGAAATAAACGCCA-3’, | 16 |
| | | 5’-TGGCAAATTCAAAGGTGAGCGAA-3’ | 17 |
| 81E7 | Alistipes senegalensis | 5’-GGGAATAAAGCTGTTCCGATATGC-3’, | 18 |
| | | 5’-TCATGCAACATTCTTTCGTTGG-3’ | 19 |
| 81H9 | Parabacteroides gordonii | 5’-TTCACCTTCTACGGCTACTACTACG-3’, | 20 |
| | | 5’-ACATAACGATCAAGGGTGCTGAAG-3’ | 21 |
| 82A6 | Paraprevotella | 5’-GCTCTTTTTAGCCTGTATCCGGT-3’, | 22 |
| | xylaniphila | 5’-ATACGATACGAACGACCAACCT-3’ | 23 |
| 82B1 | Ruthenibacterium | 5’-TACCAATGCAAAGCGACCAA-3’, | 24 |
| | lactatiformans | 5’-CGGTTTTGTTGCCGAACTCT-3’ | 25 |
| 82F11 | Parabacteriodes | 5’-GCACAGATTCTACACTCCCCT-3’,5’- | 26 |
| | johnsonii | AGCAACGAAACAACCTGTGA-3’ | 27 |
| 82G1 | Bacteroides uniformis | 5’-TCCATGCTGAAGCGTTGAAG-3’, | 28 |
| | | 5’-GGACCGAACATCCCAATCAC-3’ | 29 |
| 82G5 | Phascolarctobacterium | 5’-CTGCTTCCGACAGCACACAT-3’, | 30 |
| | succinatutens | 5’-AGCTTGGCGGAGAGCCTATT-3’ | 31 |
| 82G9 | Parabacteroides | 5’-AGCGCGTAAACTTAGTCAAGGA-3’, | 32 |
| | distasonis | 5’-TAGGGCCAAAACCTGCATTA-3’ | 33 |
| LA | Lactobacillus acidophilus | 5’-AGCAGTAGGGAATCTTCCA-3’, | 34 |
| | | 5’-CACCGCTACACATGGAG-3’ | 35 |

*Genome size: 81A6, 3.8 Mb; 81B11, 5.63 Mb; 81C1, 4.75 Mb; 81E7, 3.65 Mb; 81H9, 6.72 Mb; 82A6, 4.1 Mb; 82B1, 4.4 Mb; 82F11, 4.8M b; 82G1, 5.15 Mb; 82G5, 2.54 Mb; 82G9, 5.17 Mb based on average of hybrid nanopore and Illumina sequencing; LA, 1.97 Mb from https://www.ncbi.nlm.nih.gov/genome.

### Example 2: Microbiome Recovery and Colonization Dynamics

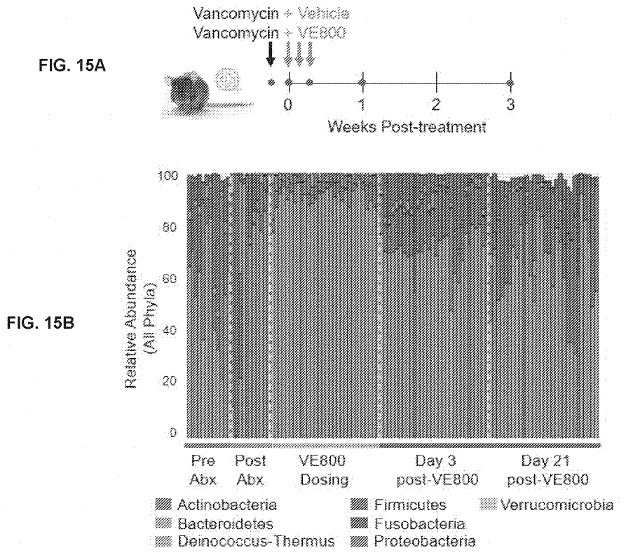

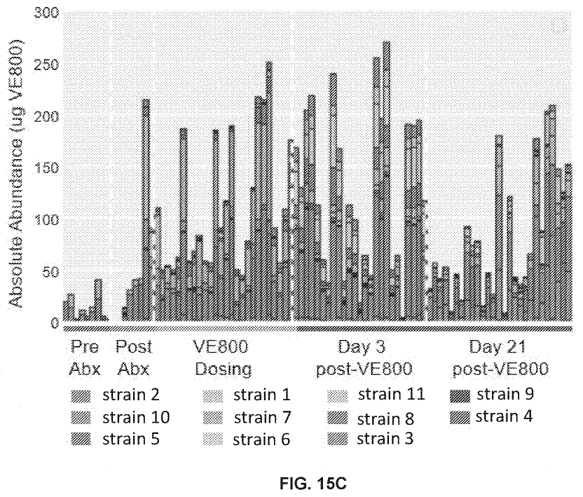

Treatment with antibiotics alters the composition of the gut microbiome, and recovery of the pre-treatment composition may restore overall health. To evaluate the effects of bacterial compositions such as VE800 on the gut microbiome following antibiotic treatment, specific pathogen free (SPF) mice were administered antibiotics (vancomycin) and subsequently administered bacterial composition VE800 or vehicle control (FIG. 15A). Samples were collected at various time points, and whole genome sequencing was performed.

FIG. 15B shows the relative abundance of all bacterial phyla in the gut microbiome prior to antibiotic treatment, post-antibiotic treatment, at the time of VE800 dosing, and at both 3 and 21 days post-VE800 dosing. Colonization of bacterial strains of the VE800 composition at various time points are shown in FIG. 15C. Administration of bacterial composition VE800 promoted the recovery of the gut microbiome after antibiotic treatment.

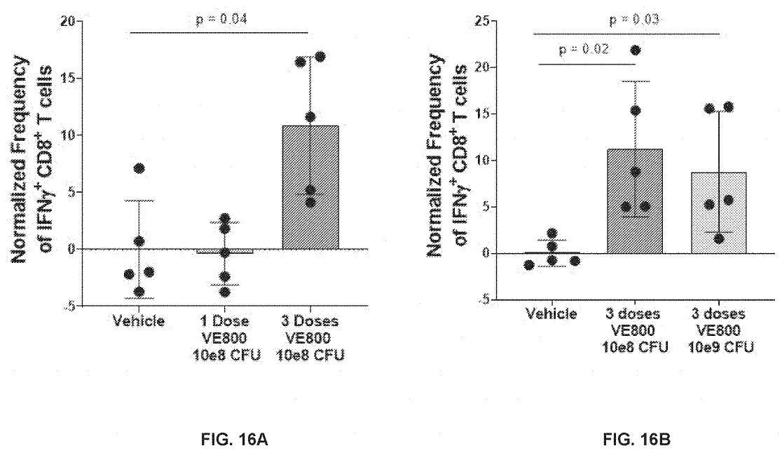

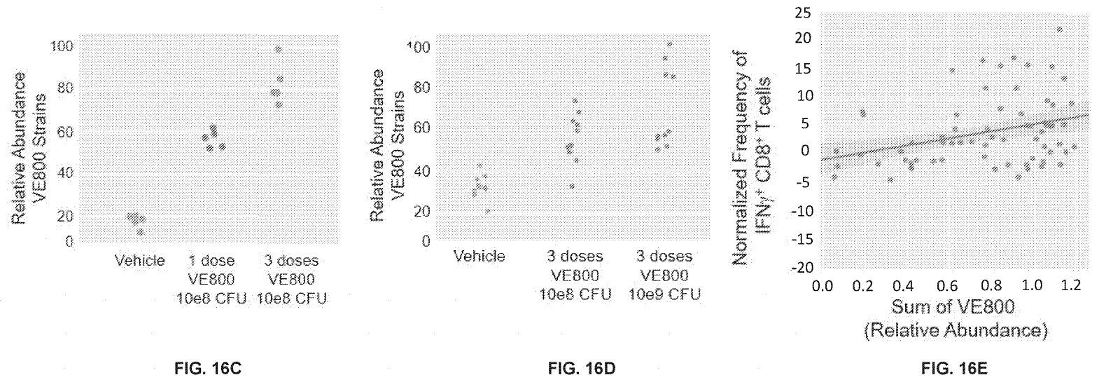

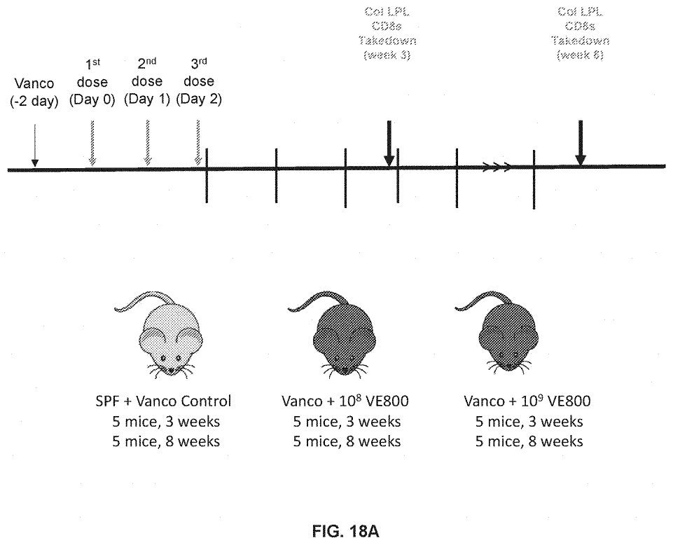

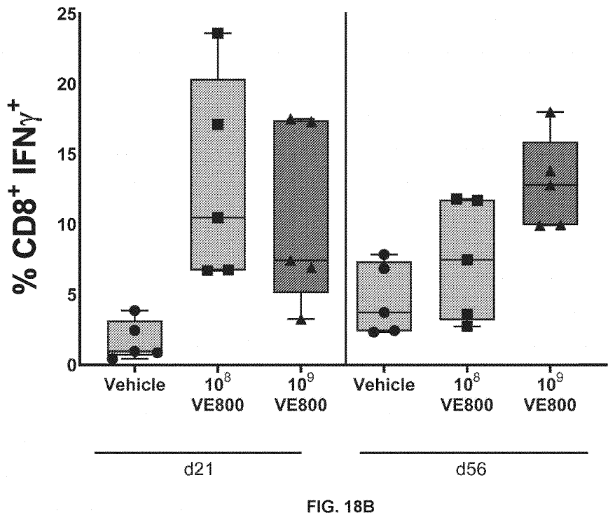

To explore colonization dynamics and the effects of the number of dosages of bacterial compositions and the amount of bacteria administered per dose, specific pathogen free mice were administered antibiotics (vancomycin) prior to administration of one or more doses of bacterial composition VE800 at defined CFU per dose. Samples were collected 3 weeks after VE800 administration, and IFNγ induction in colonic CD8⁺ T cells was measured as the frequency of IFNγ+CD8⁺ T cells. 3 doses of VE800 (10e8 colony forming units (CFU) or 10e9 CFU) were found to induce increased levels of IFNγ⁺ CD8⁺ T cells as compared to 1 dose of VE800 (10e8 CFU) or vehicle control (FIGS. 16A, 16B). These results were observed at shorter time points (e.g., day 21) as well as longer time points (e.g., day 56) (FIGS. 18A and 18B). Additionally, administration of 3 doses of VE800 (10e8 CFU or 10e9 CFU) resulted in increased relative abundance of the bacterial strains of composition VE800 as compared to 1 dose of VE800 (10e8 CFU) or vehicle control (FIGS. 16C and 16D). Regression analysis indicated that the induction of colonic IFNγ⁺ CD8⁺ T cells was correlated with the relative abundance of the bacterial strains of composition VE800 (FIG. 16E).

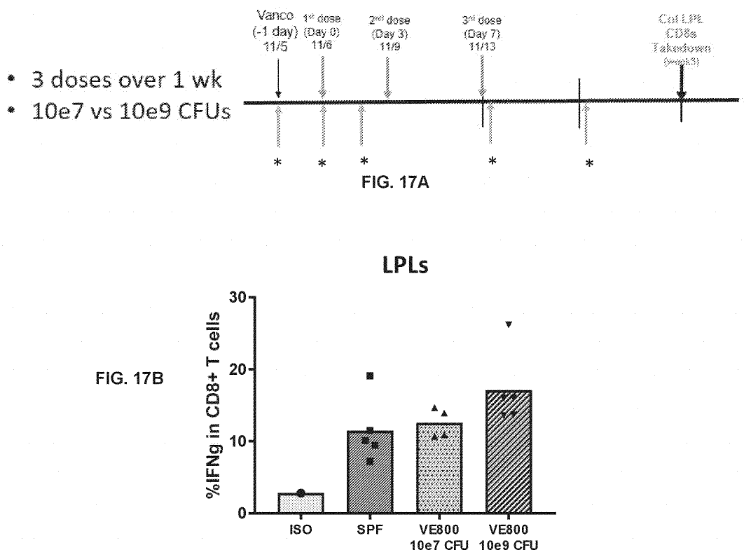

In addition, administration of doses containing 10⁷ CFU was compared to administration of doses containing 10⁹ CFU, as shown in FIG. 17A. Specific pathogen free mice were administered antibiotics (vancomycin) and subsequently administered 3 doses of 10⁷ CFU or 10⁹ CFU of bacterial composition VE800 over one week. Mice were sacrificed 3 weeks after the first dose, and colonic lamina propria lymphocytes (LPLs) were isolated and the induction of IFNγ⁺ CD8⁺ T cells was measured. Administration of 3 doses of 10⁹ CFUs of composition VE800 was found to induce increased levels of IFNγ⁺ CD8⁺ T cells as compared to 3 doses of 10⁷ CFUs of composition VE800 or control (FIG. 17B).

### Example 3: A Rationally-Designed Consortium of Human Gut Commensals Induces IFNγ+ CD8+ T Cells and Enhances Tumor Immunity

### Background

Efficacy of immune checkpoint inhibitor (ICI) therapy is associated with the composition of the gut microbiome, but the mechanism remains to be elucidated. Recent data from Tanoue et al. (see, e.g., Tanoue et al., Nature (2019) 565: 600-605) demonstrated a consortium of 11 bacteria (VE800) induced IFNγ production in intestinal CD8+ T cells and enhanced anti-PD-1 cancer activity in tumor models. VE800 has been developed as a microbiome therapeutic to enhance the efficacy of ICI therapy. There is a need to further understand the requirements for VE800 efficacy in pre-clinical models and to understand how immune modulation by VE800 affects tumor immunity.

### Methods

IFNγ production in colonic T cells was used as a readout for VE800 anti-cancer efficacy. Mice were pre-treated with different antibiotics to establish a permissive niche for colonization and given VE800. To determine which bacteria were critical contributors to VE800 function, various combinations of individual VE800 bacterial strains were created and induction of IFNγ+ CD8+ T cells in the intestines of germ-free mice was measured. Consortia unable to induce IFNγ+ CD8+ T cells were tested for their anti-tumor efficacy with ICI in the B16 tumor model. In this model, C57BL/6 mice were pre-treated with antibiotics to deplete endogenous microbiota, injected with B16, and treated with ICI on days 4, 8, and 12. VE800 was administered three times a week for three weeks prior to sacrifice to assess levels of tumor infiltrating cells.

### Results

The data suggest that bacterial composition and density drive VE800 efficacy for induction of IFNγ+ CD8+ T cells. The complete VE800 consortium provides maximal IFNγ induction, and removal of specific strains impairs IFNγ induction. Utilizing the B16 tumor model, colonization of VE800 strains was observed in tumor-bearing mice receiving the complete consortia. Additionally, the complete VE800 consortium restored ICI efficacy in antibiotic-treated tumor-bearing mice. However, bacterial consortia incapable of inducing IFNγ+ CD8+ T cells do not promote anti-tumor immunity. Pre-treatment with antibiotics abolished the therapeutic effect of ICI, which was accompanied by a reduced level of tumor infiltrating immune cells indicating the important, supportive role played by gut microbiota.

### Conclusions

The data suggests that VE800 induces IFNγ+ CD8+ T cell induction and provides the necessary microbial component to promote anti-tumor immunity in combination with dual checkpoint inhibitor therapy in the multiple tumor models.

### Example 4: Precision Probiotic Therapy Enhances Immune Checkpoint Inhibitor Therapy Efficacy in Melanoma Bearing Mice

### Background

Immune checkpoint inhibitor therapy (ICT) achieves remission in melanoma patients, but factors modulating this response are not well-defined. Specific gut microbiota have been identified that are associated with improved ICT response and that induce adaptive immune responses and potentiate ICT (see, e.g., Frankel et al. Neoplasia (2017) and Tanoue et al. Nature (2019)). This study explored whether the predefined consortia of gut microbiota augments ICT efficacy in melanoma bearing mice.

### Methods

Mice (C57BL/6, 6-8 wk old, female, Jackson, n=4-12 mice) received water with or without antibiotics (penicillin G 1500 U/mL+streptomycin 2 mg/mL) for 6 days to deplete gut microbiota. Mice were then inoculated with 10⁵ B16F10 melanoma cells SQ. At days 4, 8, 12 post-tumor inoculation, 0.2 mg anti-mCTLA4+anti-mPD1 antibodies (Bio X Cell) were administered intraperitoneally (IP). Precision probiotic therapies including VE800 (Tanoue et al., Nature 2019), VE804 (same as VE800 without Ruminococcus lactatiformans and Fusobacterium ulcerans), VE411 (four Clostridial firmicutes) (Narushima, et al. Gut Microbes (2014)5(3): 333-9), and Lactobacillus acidophilus (ATCC 4356) probiotics were given via gavage (1×10⁹ colony forming units (CFUs)) starting one day after tumor inoculation 3 times weekly. Loss of survival was defined as death or tumor diameters ≥2 cm. Tumor growth inhibition, TGI=(1−mean treated tumor volume/mean control tumor volume)×100%. Tumor mononuclear cells were isolated for flow cytometry for murine CD4, CD8, and CD11c.

### Results

Tumor growth inhibition (TGI) in mice with intact gut microbiota and treated with ICT was 84±4% (standard error of the mean). Pre-treatment antibiotics reduced TGI to 38±11%. Groups treated with VE800, VE804, and VE411 exhibited TGIs of 77±9, 61±8, and 69%, respectively, whereas treatment with Lactobacillus acidophilus achieved TGIs 57%. VE800 treated mice had significantly increased length of survival compared to mice treated with antibiotics (p=0.0008, log-rank test). Length of survival was not significantly different between groups with intact gut microbiota and those pretreated with antibiotics and dosed with VE800 (p=0.52, log-rank test). ICT increased tumor CD4 cells to 11% from 2% and CD8 cells to 9% from 1%, however pre-treatment with antibiotics reduced CD4 cells to 4% and CD8 cells to 1%.

### Conclusions

Defined consortia of gut microbiota facilitate ICT efficacy. These preclinical studies lay the foundation for optimizing the host response to ICT.

### Example 5: Safety and Efficacy Study of VE800 in Combination with Nivolumab in Previously Treated Patients with Select Advanced Metastatic Cancers

### Background

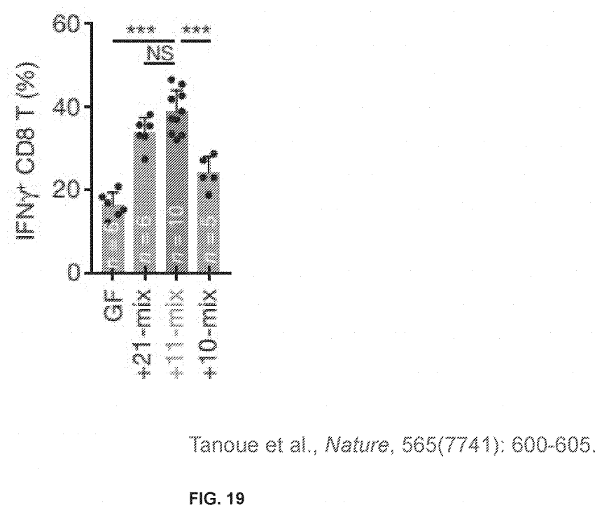

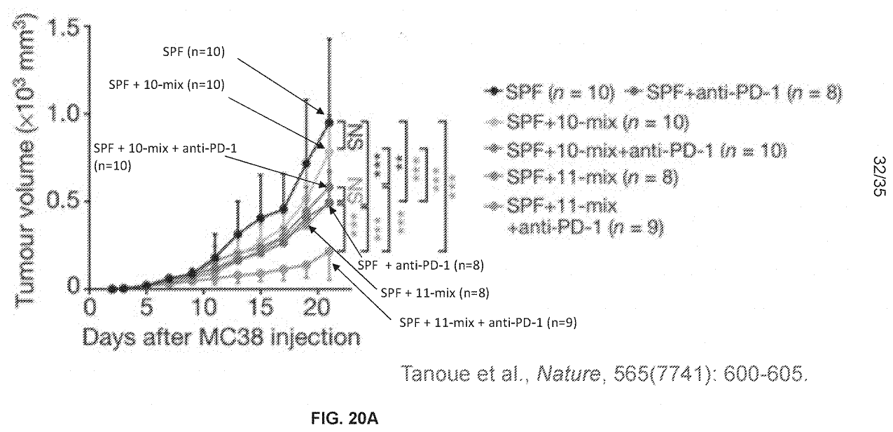

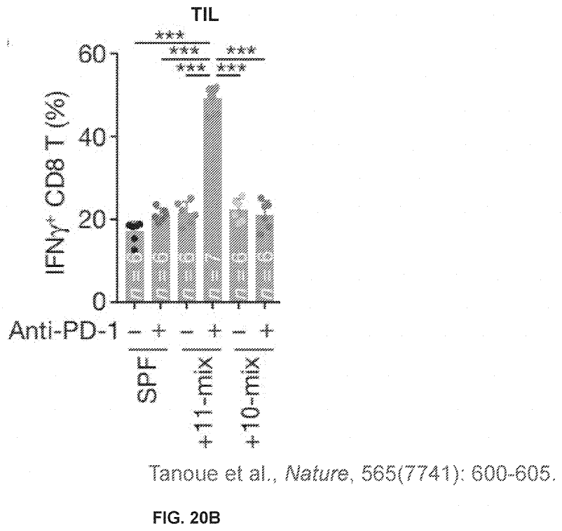

Gut microbiome composition affects the response to PD-1 blockade. Recent proof-of-concept studies suggest that gut microbiome manipulation is effective in reversing resistance to PD-1 blockade. VE800 is an oral live biotherapeutic consisting of 11 distinct non-pathogenic, non-toxigenic, commensal bacterial strains manufactured in lyophilized form. VE800 induces proliferation of IFNγ+ CD8+ T cells (FIG. 19), reduction in tumor volume (FIG. 20A), CD8+ T cell infiltrate into tumors (FIG. 20B), and significantly enhances anti-tumor activity of PD-1 blockade preclinically in multiple tumor models in a CD103+ dendritic cell and major histocompatibility (MHC) class I-dependent fashion (see, Tanoue et al., Nature (2019) 565: 600-605). Retrospective analysis of cancer patient's samples also suggests that greater VE800 strain abundance is associated with an improved response to PD-1 blockade. Described herein is a Phase 1 study to evaluate the safety, tolerability, and clinical activity of VE800 administered orally in combination with nivolumab in patients with select cancers.

### Methods

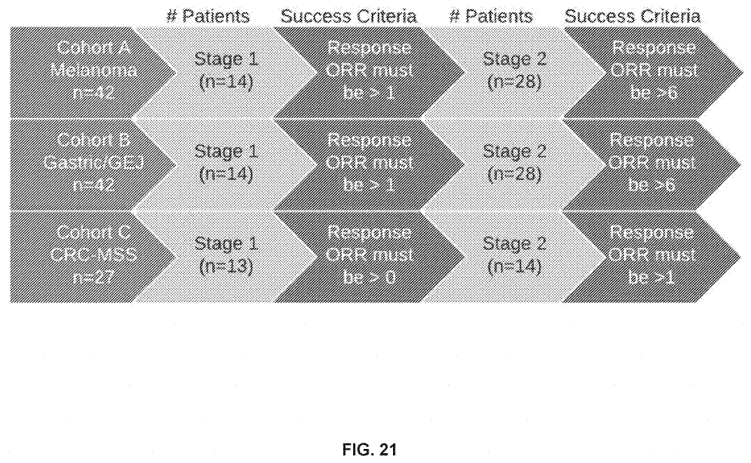

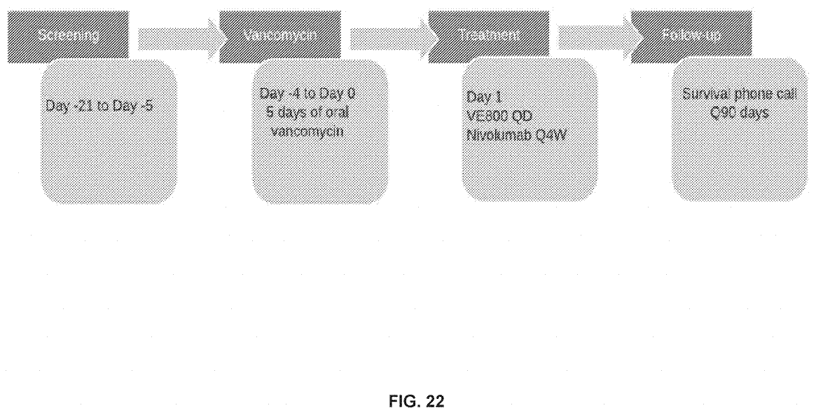

CONSORTIUM-IO (NCT4208958) is an open-label, first-in-human study evaluating VE800 and nivolumab (PD-1 inhibitor) combination in patients with anti-PD-1/PD-L1 relapsed/refractory melanoma, anti-PD-1/PD-L1 naïve gastric/gastroesophageal junction (GEJ) adenocarcinoma, and anti-PD-1/PD-L1 naïve microsatellite-stable (MSS) colorectal cancer (FIG. 21). As shown in FIG. 22, following a 5-day course of oral vancomycin 125 every day (QID), VE800 is administered daily along with nivolumab (480 mg every 4 weeks, Q4W). A single dose of VE800 is evaluated. In the interest of maximizing safety, a Simon 2-stage design is used for each disease cohort (FIG. 21). The first three patients are enrolled serially one week apart, and safety data of the six patients are reviewed prior to further accrual. Patients continue to receive VE800/nivolumab combination until disease progression or unacceptable toxicity.

Criteria for inclusion in the study for all patients include having received ≤3 lines of systemic therapy for advanced disease, confirmed progressive disease (PD) (at least 2 consecutive scans 4 weeks apart) using standards based on Response Evaluation Criteria in Solid Tumors (RECIST v 1.1), and adequate organ function. For patients with melanoma, inclusion criteria also include progression on/within 3 months of anti PD-1/PD-L1 therapy singly or in combination and use of anti-PD-1/PD-L1 as the most recent therapy received. For gastric/GEJ patients, inclusion criteria also include progression after ≥1 line of therapy including fluoropyrimidine/platinum and HER2/neu-targeted therapy (if appropriate). For CRC-MSS patients, inclusion criteria also include progression after ≥1 line of therapy including fluoropyrimidine, oxaliplatin, or irinotecan (unless contraindicated).

Exclusion criteria related to the nature of the VE800 composition include having a prior total colectomy or current ileostomy, and having celiac disease, irritable bowel syndrome (IBS), short gut, or current gastrointestinal fistulas, or ischemia. Exclusion criteria related to the mechanism of action of the combination of VE800 and the PD-1 inhibitor include prior treatment with an immune checkpoint inhibitor (except for melanoma patients), a history of life-threatening toxicity related to prior immune therapy, having an autoimmune disease, administration of corticosteroids or immunosuppressive medications within 14 days of initiation of the trial. Prohibited therapies include administration of a live/attenuated vaccine within the 30 days prior to administration of VE800; use of immunosuppressive agents (excluding steroids for physiologic repletion), administration of fecal microbial products or fecally-derived products or live bacterial products (including prior FMT). Further, use of antibiotics with significant impact on the intestinal microbiota should be avoided during the study period, unless clinically indicated.

The study involves biopsy (archival is required), and a fresh biopsy is recommended prior to initiation of VE800 dosing and after 4-6 weeks of treatment. RECIST 1.1 parameters are used to assess the response in addition to confirmatory scans.

Primary endpoints include safety, tolerability, and clinical activity by objective response rate (ORR) per RECIST v1.1. Secondary endpoints include duration of response (DOR), disease control rate (DCR), progression-free survival (PFS), overall survival (OS), and metagenomic strain-level analysis of degree/duration of VE800 strain colonization. Exploratory endpoints include tumor and blood immunophenotyping, serum/stool metabolomics, and global changes in fecal microbiome composition.

The dose limiting toxicity (DLT) period is 28 days. DLT criteria include a grade 3 or grade 4 immune-mediated adverse event or any grade immune-mediated adverse event requiring treatment discontinuation; greater than or equal to grade 2 uveitis or eye pain; grade 2 or grade 3 colitis or diarrhea; any other grade 3 or higher non-hematologic clinical toxicity; and any clinically significant hematologic toxicity.

Follow-up with the patients is performed every 90 days to assess survival.

What is claimed is:

1. A composition comprising Eubacterium limosum, Subdolinogranulum sp., Phascolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis.

2. A composition comprising Fusobacterium ulcerans, Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

3. A composition comprising Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

4. A composition comprising Fusobacterium ulcerans, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

5. A composition comprising Fusobacterium ulcerans, Subdolinogranulum sp., Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

6. A composition consisting of Eubacterium limosum, Subdolinogranulum sp., Phascolarctobacterium faecium, Parabacteriodes johnsonii, Paraprevotella xylaniphila, Parabacteroides distasonis, Alistipes sp., Bacteroides dorei, Parabacteroides gordonii, and Bacteroides uniformis.

7. A composition consisting of Fusobacterium ulcerans, Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

8. A composition consisting of Eubacterium limosum, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

9. A composition consisting of Fusobacterium ulcerans, Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

10. A composition consisting of Fusobacterium ulcerans, Subdolinogranulum sp., Phascolarctobacterium faecium, Parabacteriodes johnsonii, Paraprevoella xylaniphila, Parabacteroides distasonis, Alistipes sp., Bacteroides dorei, Parabacteroides gordonii, and Bacteroides uniformis.

11. A composition comprising a purified bacterial mixture comprising bacterial strains comprising 16S rDNA sequences of at least 97% sequence identity to the nucleic acid sequences set forth as SEQ ID NOs: 1 and 3-11.

12. A composition comprising a purified bacterial mixture comprising bacterial strains comprising 16S rDNA sequences of at least 97% sequence identity to the nucleic acid sequences set forth as SEQ ID NOs: 1-4 and 6-11.

13. A composition comprising a purified bacterial mixture comprising bacterial strains comprising 16S rDNA sequences of at least 97% sequence identity to the nucleic acid sequences set forth as SEQ ID NOs: 1, 3, 4, and 6-11.

14. A composition comprising a purified bacterial mixture comprising bacterial strains comprising 16S rDNA sequences of at least 97% sequence identity to the nucleic acid sequences set forth as SEQ ID NOs: 1-4, 6-9, and 11.

15. A composition comprising a purified bacterial mixture comprising bacterial strains comprising 16S rDNA sequences of at least 97% sequence identity to the nucleic acid sequences set forth as SEQ ID NOs: 1-9 and 11.

16. A composition comprising a purified bacterial mixture consisting of bacterial strains comprising 16S rDNA sequences of at least 97% sequence identity to the nucleic acid sequences set forth as SEQ ID NOs: 1 and 3-11.

17. A composition comprising a purified bacterial mixture consisting of bacterial strains comprising 16S rDNA sequences of at least 97% sequence identity to the nucleic acid sequences set forth as SEQ ID NOs: 1-4 and 6-11.

18. A composition comprising a purified bacterial mixture consisting of bacterial strains comprising 16S rDNA sequences of at least 97% sequence identity to the nucleic acid sequences set forth as SEQ ID NOs: 1, 3, 4, and 6-11.

19. A composition comprising a purified bacterial mixture consisting of bacterial strains comprising 16S rDNA sequences of at least 97% sequence identity to the nucleic acid sequences set forth as SEQ ID NOs: 1-4, 6-9, and 11.

20. A composition comprising a purified bacterial mixture consisting of bacterial strains comprising 16S rDNA sequences of at least 97% sequence identity to the nucleic acid sequences set forth as SEQ ID NOs: 1-9 and 11.

21. The composition of any one of claims 1-20, wherein the composition is effective in enhancing immune checkpoint inhibitor therapy.

22. The composition of any one of claims 1-21, wherein the composition is effective in treating colitis associated with checkpoint inhibitor therapy.

23. The composition of any one of claims 1-22, wherein the composition is effective in inducing the production of regulatory T cells (Tregs) in the intestine.

24. The composition of any one of claims 1-23, wherein the composition is effective in inducing the production of IFNγ+ CD8+ T cells in the intestine.

25. The composition of any one of claims 1-24, wherein the bacterial strains are lyophilized.

26. The composition of any one of claims 1-25, wherein the bacterial strains are spray-dried.

27. The composition of any one of claims 1-26, wherein one or more of the bacterial strains are in spore form.

28. The composition of any one of claims 1-27, wherein each of the bacterial strains is in spore form.

29. The composition of any one of claims 1-27, wherein one or more of the bacterial strains are in vegetative form.

30. The composition of any one of claim 1-26 or 29, wherein each of the bacterial strains is in vegetative form.

31. The composition of any one of claims 1-30, wherein the composition further comprises one or more anticancer agents.

32. The composition of claim 31, wherein the anticancer agent is a chemotherapy agent.

33. The composition of claim 32, wherein the anticancer agent is a cancer immunotherapy agent.

34. The composition of claim 33, wherein the cancer immunotherapy agent is an immune checkpoint inhibitor.

35. The composition of claim 34, wherein the immune checkpoint inhibitor is a PD-1 inhibitor, PD-L-1 inhibitor, or CTLA-4 inhibitor.

36. The composition of claim 35, wherein the immune checkpoint inhibitor is a PD-1 inhibitor.

37. The composition of claim 36, wherein the PD-1 inhibitor is nivolumab.

38. The composition of claim 36, wherein the PD-1 inhibitor is pembrolizumab.

39. The composition of claim 36, wherein the PD-1 inhibitor is pidiluzimab.

40. The composition of claim 35, wherein the immune checkpoint inhibitor is a PD-L-1 inhibitor.

41. The composition of claim 40, wherein the PD-L-1 inhibitor is avelumab.

42. The composition of claim 40, wherein the PD-L-1 inhibitor is durvalumab.

43. The composition of claim 35, wherein the immune checkpoint inhibitor is a CTLA-4 inhibitor.

44. The composition of claim 43, wherein the CTLA-4 inhibitor is an anti-CTLA-4 antibody.

45. The composition of claim 43, wherein the CTLA-4 inhibitor is ipilimumab.

46. The composition of claim 43, wherein the CTLA-4 inhibitor is tremelimumab.

47. A pharmaceutical composition comprising the composition of any one of claims 1-46, further comprising a pharmaceutically acceptable excipient.

48. The pharmaceutical composition of claim 47, wherein the pharmaceutical composition is formulated for oral delivery.

49. The pharmaceutical composition of claim 47, wherein the pharmaceutical composition is formulated for rectal delivery.

50. The pharmaceutical composition of any one of claims 47-49, wherein the pharmaceutical composition is formulated for delivery to the intestine.

51. The pharmaceutical composition of any one of claims 47-50, wherein the pharmaceutical composition is formulated for delivery to the colon.

52. The pharmaceutical composition of any one of claims 47-51, wherein the pharmaceutical composition is administered as one dose.

53. The pharmaceutical composition of any one of claims 47-51, wherein the pharmaceutical composition is administered as multiple doses.

54. The pharmaceutical composition of claim 52 or 53, wherein each dose comprises the administration of multiple capsules.

55. A food product comprising the composition of any one of claims 1-46 and a nutrient.

56. A method for treating cancer in a subject, the method comprising administering to the subject the composition of any one of claims 1-54 or the food product of claim 55 in an effective amount to treat the cancer.

57. The method of claim 56, wherein the cancer is carcinoma, glioma, mesothelioma, melanoma, lymphoma, leukemia, adenocarcinoma, breast cancer, ovarian cancer, cervical cancer, glioblastoma, multiple myeloma, prostate cancer, Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumors, or rhabdomyosarcoma.

58. The method of claim 57, wherein the cancer is prostate cancer, bladder cancer, non-small cell lung cancer, urothelial carcinoma, melanoma, or renal cell carcinoma.

59. The method of any one of claims 56-58, wherein an antibiotic is administered prior to the administration of any one of the compositions.

60. The method of any one of claims 56-59, wherein the subject is human.

61. The method of any one of claims 56-60, wherein the composition is administered to the subject more than once.

62. The method of any one of claims 56-61, wherein the composition is administered to the subject by oral administration.

63. The method of any one of claims 56-61, wherein the composition is administered to the subject by rectal administration.

64. The method of any one of claims 56-63, further comprising administering an antibiotic to the subject prior to administration of any one of the compositions or food products.

65. The method of any one of claims 55-64, wherein any one of the compositions or food products is administered to the subject in multiple doses.

66. The method of claim 65, wherein if one or more bacterial strains of the compositions colonize the subject to a level above a threshold level, no further compositions or food products are administered to the subject.

67. The method of claim 66, wherein if the compositions induce a CD8+ T cell response above a threshold level, no further compositions or food products are administered to the subject.

68. A method of treating colitis associated with checkpoint inhibitor therapy in a subject, comprising administering to the subject a therapeutically effective amount of the composition of any one of claims 1-54 or the food product of claim 55.

69. The method of claim 68, wherein the subject is human.

70. The method of claim 68 or 69, wherein the composition is administered to the subject more than once.

71. The method of any one of claims 68-70, wherein the composition is administered to the subject by oral administration.

72. The method of any one of claims 68-70, wherein the composition is administered to the subject by rectal administration.

73. The method of any one of claims 68-72, further comprising administering an antibiotic to the subject prior to administration of any one of the compositions or food products.

74. The method of any one of claims 68-73, wherein any one of the compositions or food products is administered to the subject in multiple doses.

75. The method of claim 74, wherein if one or more bacterial strains of the compositions colonize the subject to a level above a threshold level, no further compositions or food products are administered to the subject.

76. The method of claim 74, wherein if the compositions induce a CD8+ T cell response above a threshold level, no further compositions or food products are administered to the subject.

77. A method for colonizing a microbiome of a subject, the method comprising administering to the subject a composition of any one of claims 1-54 or the food product of claim 55 in an effective amount to colonize the microbiome.

78. The method of claim 77, wherein the subject is human.

79. The method of claim 77 or 78, wherein the composition is administered to the subject more than once.

80. The method of any one of claims 77-79, wherein the composition is administered to the subject by oral administration.

81. The method of any one of claims 77-79, wherein the composition is administered to the subject by rectal administration.

82. The method of any one of claims 77-81, further comprising administering an antibiotic to the subject prior to administration of any one of the compositions or food products.

83. The method of any one of claims 77-82, wherein any one of the compositions or food products is administered to the subject in multiple doses.

84. The method of claim 83, wherein if one or more bacterial strains of the compositions colonize the subject to a level above a threshold level, no further compositions or food products are administered to the subject.

85. The method of claim 83, wherein if the compositions induce a CD8+ T cell response above a threshold level, no further compositions or food products are administered to the subject.

86. A method for restoring a microbiome in a subject, the method comprising administering to the subject a composition of any one of claims 1-54 or the food product of claim 55 in an effective amount to restore the microbiome in the subject.

87. The method of claim 86, wherein the subject has not undergone a dysbiosis inducing event.

88. The method of claim 86 or 87, wherein the subject has not had an infectious disease.

89. The method of any one of claims 86-88, wherein the subject has not been treated with an antibiotic.

90. The method of claim 89, wherein the subject has undergone a dysbiosis inducing event.

91. The method of claim 90, wherein the dysbiosis inducing event is treatment with one or more antibiotic.

92. The method of any one of claims 86-91, wherein the subject is human.

93. The method of any one of claims 86-92, wherein the composition is administered to the subject more than once.

94. The method of any one of claims 86-93, wherein the composition is administered to the subject by oral administration.

95. The method of any one of claims 86-93, wherein the composition is administered to the subject by rectal administration.

96. The method of any one of claims 86-95, further comprising administering an antibiotic to the subject prior to administration of any one of the compositions or food products.

97. The method of any one of claims 86-96, wherein any one of the compositions or food products is administered to the subject in multiple doses.

98. The method of claim 97, wherein if one or more bacterial strains of the compositions colonize the subject to a level above a threshold level, no further compositions or food products are administered to the subject.

99. The method of claim 97, wherein if the compositions induce a CD8+ T cell response above a threshold level, no further compositions or food products are administered to the subject.
